SEQUENCE LISTING (73 sequences)

SEQ ID NO: 1 - DNA, 268 bp, Homo sapiens
tcggaccgcggcaactacacctgcgtcgtggagaacaagtttggcagcatccggcagacgtacacgctggacgtgctggagtgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctca

SEQ ID NO: 2 - DNA, 378 bp, Homo sapiens
gaccgcggcaactacacctgcgtcgtggagaacaagtttggcagcatccggcagacgtacacgctggacgtgctgggtgagggccctggggcggcgcgggggtgggggcggcagtggcggtggtggtgagggagggggtggcccctgagcgtcatctgcccccacagagcgctgcccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgggccaccgtgtgcacgt

SEQ ID NO: 3 - DNA, 234 bp, Homo sapiens
gcgggcaattctattgggttttctcatcactctgcgtggctggtggtgctgccagccgaggaggagctggtggaggctgacgaggcgtgcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccg

SEQ ID NO: 4 - DNA, 301 bp, Homo sapiens
ctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtgtgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagc

SEQ ID NO: 5 - DNA, 21 bp, Artificial Sequence
  Description of Artificial Sequence Synthetic primer
gacctggaccgtgtccttacc

SEQ ID NO: 6 - DNA, 21 bp, Artificial Sequence
  Description of Artificial Sequence Synthetic primer
cttccccagttccaggttctt

SEQ ID NO: 7 - DNA, 20 bp, Artificial Sequence
  Description of Artificial Sequence Synthetic primer
aggacctggaccgtgtcctt

SEQ ID NO: 8 - DNA, 20 bp, Artificial Sequence
  Description of Artificial Sequence Synthetic primer
tataggtccggtggacaggg

SEQ ID NO: 9 - DNA, 15 bp, Artificial Sequence
  Description of Artificial Sequence Synthetic primer
ggccatcctgccccc

SEQ ID NO: 10 - DNA, 20 bp, Artificial Sequence
  Description of Artificial Sequence Synthetic primer
gagcagtccaggtcagccag

SEQ ID NO: 11 - DNA, 20 bp, Artificial Sequence
  Description of Artificial Sequence Synthetic primer
ctggaccgtgtccttaccgt

SEQ ID NO: 12 - DNA, 20 bp, Artificial Sequence
  Description of Artificial Sequence Synthetic primer
gcagcccaggattgaactgt

SEQ ID NO: 13 - DNA, 23 bp, Artificial Sequence
  Description of Artificial Sequence Synthetic primer
tggatcgaattctcactctcaca

SEQ ID NO: 14 - DNA, 21 bp, Artificial Sequence
  Description of Artificial Sequence Synthetic primer
gccaagcaatctgcgtatttg

SEQ ID NO: 15 - DNA, 25 bp, Artificial Sequence
  Description of Artificial Sequence Synthetic primer
tggtagaagacttggatcgaattct

SEQ ID NO: 16 - DNA, 23 bp, Artificial Sequence
  Description of Artificial Sequence Synthetic primer
tctcccggattatttcttcaaca

SEQ ID NO: 17 - DNA, 23 bp, Artificial Sequence
  Description of Artificial Sequence Synthetic primer
gctcttcaatacagccctgatca

SEQ ID NO: 18 - DNA, 24 bp, Artificial Sequence
  Description of Artificial Sequence Synthetic primer
acttggatcgaattctcactctca

SEQ ID NO: 19 - DNA, 23 bp, Artificial Sequence
  Description of Artificial Sequence Synthetic primer
tggatcgaattctcactctcaca

SEQ ID NO: 20 - DNA, 25 bp, Artificial Sequence
  Description of Artificial Sequence Synthetic primer
gcaaagcctgaattttcttgaataa

SEQ ID NO: 21 - DNA, 24 bp, Artificial Sequence
  Description of Artificial Sequence Synthetic primer
agggtgcatcaactcatgaattag

SEQ ID NO: 22 - DNA, 24 bp, Artificial Sequence
  Description of Artificial Sequence Synthetic primer
acttggatcgaattctcactctca

SEQ ID NO: 23 - DNA, 18 bp, Artificial Sequence
  Description of Artificial Sequence Synthetic primer
gcatccggcagacgtaca

SEQ ID NO: 24 - DNA, 15 bp, Artificial Sequence
  Description of Artificial Sequence Synthetic primer
ccccgcctgcaggat

SEQ ID NO: 25 - DNA, 18 bp, Artificial Sequence
  Description of Artificial Sequence Synthetic primer
gcatccggcagacgtaca

SEQ ID NO: 26 - DNA, 15 bp, Artificial Sequence
  Description of Artificial Sequence Synthetic primer
ccccgcctgcaggat

SEQ ID NO: 27 - DNA, 19 bp, Artificial Sequence
  Description of Artificial Sequence Synthetic primer
aggagctggtggaggctga

SEQ ID NO: 28 - DNA, 19 bp, Artificial Sequence
  Description of Artificial Sequence Synthetic primer
ccgtagctgaggatgcctg

SEQ ID NO: 29 - DNA, 18 bp, Artificial Sequence
  Description of Artificial Sequence Synthetic primer
ctggtggaggctgacgag

SEQ ID NO: 30 - DNA, 16 bp, Artificial Sequence
  Description of Artificial Sequence Synthetic primer
agcccaccccgtagct

SEQ ID NO: 31 - DNA, 21 bp, Artificial Sequence
  Description of Artificial Sequence Synthetic primer
gtcgtggagaacaagtttggc

SEQ ID NO: 32 - DNA, 17 bp, Artificial Sequence
  Description of Artificial Sequence Synthetic primer
gtctggttggccggcag

SEQ ID NO: 33 - DNA, 21 bp, Artificial Sequence
  Description of Artificial Sequence Synthetic primer
gtcgtggagaacaagtttggc

SEQ ID NO: 34 - DNA, 17 bp, Artificial Sequence
  Description of Artificial Sequence Synthetic primer
gtctggttggccggcag

SEQ ID NO: 35 - DNA, 19 bp, Artificial Sequence
  Description of Artificial Sequence Synthetic primer
aggagctggtggaggctga

SEQ ID NO: 36 - DNA, 19 bp, Artificial Sequence
  Description of Artificial Sequence Synthetic primer
ccgtagctgaggatgcctg

SEQ ID NO: 37 - DNA, 16 bp, Artificial Sequence
  Description of Artificial Sequence Synthetic primer
gacgaggcgggcagtg

SEQ ID NO: 38 - DNA, 19 bp, Artificial Sequence
  Description of Artificial Sequence Synthetic primer
gaagaagcccaccccgtag

SEQ ID NO: 39 - DNA, 17 bp, Artificial Sequence
  Description of Artificial Sequence Synthetic oligonucleotide
tggagcgctccccgcac

SEQ ID NO: 40 - DNA, 18 bp, Artificial Sequence
  Description of Artificial Sequence Synthetic oligonucleotide
gacgtgctggagygctcc

SEQ ID NO: 41 - DNA, 17 bp, Artificial Sequence
  Description of Artificial Sequence Synthetic oligonucleotide
ctgacgaggcgggcagc

SEQ ID NO: 42 - DNA, 23 bp, Artificial Sequence
  Description of Artificial Sequence Synthetic oligonucleotide
gtgtgtatgcaggcatcctcagc

SEQ ID NO: 43 - DNA, 16 bp, Artificial Sequence
  Description of Artificial Sequence Synthetic probe
tccaccgacgtaaagg

SEQ ID NO: 44 - DNA, 16 bp, Artificial Sequence
  Description of Artificial Sequence Synthetic probe
tccaccgacgtgccag

SEQ ID NO: 45 - DNA, 18 bp, Artificial Sequence
  Description of Artificial Sequence Synthetic probe
ccaatgagatcatggagg

SEQ ID NO: 46 - DNA, 16 bp, Artificial Sequence
  Description of Artificial Sequence Synthetic probe
ccttctggcccaggtg

SEQ ID NO: 47 - DNA, 17 bp, Artificial Sequence
  Description of Artificial Sequence Synthetic probe
caccgacaatgttatgg

SEQ ID NO: 48 - DNA, 20 bp, Artificial Sequence
  Description of Artificial Sequence Synthetic probe
tcacaaccaatgaggagagt

SEQ ID NO: 49 - DNA, 16 bp, Artificial Sequence
  Description of Artificial Sequence Synthetic probe
ctgccatctcattggt

SEQ ID NO: 50 - DNA, 17 bp, Artificial Sequence
  Description of Artificial Sequence Synthetic probe
aatgagcaagccagggc

SEQ ID NO: 51 - DNA, 19 bp, Artificial Sequence
  Description of Artificial Sequence Synthetic probe
aagttgtgtctcattggtt

SEQ ID NO: 52 - DNA, 14 bp, Artificial Sequence
  Description of Artificial Sequence Synthetic probe
ctggagtgctcccc

SEQ ID NO: 53 - DNA, 13 bp, Artificial Sequence
  Description of Artificial Sequence Synthetic probe
agcgctgcccgca

SEQ ID NO: 54 - DNA, 15 bp, Artificial Sequence
  Description of Artificial Sequence Synthetic probe
gcgtgcagtgtgtat

SEQ ID NO: 55 - DNA, 15 bp, Artificial Sequence
  Description of Artificial Sequence Synthetic probe
ctgcacacacactgc

SEQ ID NO: 56 - DNA, 2850 bp, Artificial Sequence
  Description of Artificial Sequence Synthetic polynucleotide
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 57 - DNA, 2955 bp, Artificial Sequence
  Description of Artificial Sequence Synthetic polynucleotide
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 58 - DNA, 4462 bp, Artificial Sequence
  Description of Artificial Sequence Synthetic polynucleotide
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgagtgctggctctggcctggtgccacccgcctatgcccctccccctgccgtccccggccatcctgccccccagagtgctgaggtgtggggcgggcctttctggcccaggtgccctggctgacctggactgctcaagctcttcccagagcccaggaagttctgagaaccaaatggtgtctccaggaaaagtgtctggcagccctgagcaagccgtggaggaaaaccttagttcctattccttagacagaagagtgacacccgcctctgagaccctagaagacccttgcaggacagagtcccagcacaaagcggagactccgcacggagccgaggaagaatgcaaagcggagactccgcacggagccgaggaggaatgccggcacggtggggtctgtgctcccgcagcagtggccacttcgcctcctggtgcaatccctaaggaagcctgcggaggagcacccctgcagggtctgcctggcgaagccctgggctgccctgcgggtgtgggcacccccgtgccagcagatggcactcagacccttacctgtgcacacacctctgctcctgagagcacagccccaaccaaccacctggtggctggcagggccatgaccctgagtcctcaggaagaagtggctgcaggccaaatggccagctcctcgaggagcggacctgtaaaactagaatttgatgtatctgatggcgccaccagcaaaagggcacccccaccaaggagactgggagagaggtccggcctcaagcctcccttgaggaaagcagcagtgaggcagcaaaaggccccgcaggaggtggaggaggacgacggtaggagcggagcaggagaggacccccccatgccagcttctcggggctcttaccacctcgactgggacaaaatggatgacccaaacttcatcccgttcggaggtgacaccaagtctggttgcagtgaggcccagcccccagaaagccctgagaccaggctgggccagccagcggctgaacagttgcatgctgggcctgccacggaggagccaggtccctgtctgagccagcagctgcattcagcctcagcggaggacacgcctgtggtgcagttggcagccgagaccccaacagcagagagcaaggagagagccttgaactctgccagcacctcgcttcccacaagctgtccaggcagtgagccagtgcccacccatcagcaggggcagcctgccttggagctgaaagaggagagcttcagagaccccgctgaggttctaggcacgggcgcggaggtggattacctggagcagtttggaacttcctcgtttaaggagtcggccttgaggaagcagtccttatacctcaagttcgaccccctcctgagggacagtcctggtagaccagtgcccgtggccaccgagaccagcagcatgcacggtgcaaatgagactccctcaggacgtccgcgggaagccaagcttgtggagttcgatttcttgggagcactggacattcctgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 59 - DNA, 3765 bp, Artificial Sequence
  Description of Artificial Sequence Synthetic polynucleotide
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacaatgttatggaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaactgaactgggacatgtcctcatagagatttcaagtacccacaagaaactcaacgagagtcttgatgaaaattttaaaaaattccacaaagagattatccatgagctggagaagaagatagaacttgacgtgaaatatatgaacgcaactctaaaaagataccaaacagaacacaagaataaattagagtctttggagaaatcccaagctgagttgaagaagatcagaaggaaaagccaaggaagccgaaacgcactcaaatatgaacacaaagaaattgagtatgtggagaccgttacttctcgtcagagtgaaatccagaaattcattgcagatggttgcaaagaggctctgcttgaagagaagaggcgcttctgctttctggttgataagcactgtggctttgcaaaccacatacattattatcacttacagtctgcagaactactgaattccaagctgcctcggtggcaggagacctgtgttgatgccatcaaagtgccagagaaaatcatgaatatgatcgaagaaataaagaccccagcctctacccccgtgtctggaactcctcaggcttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaagaaaatgagacagaagcagtgaccgtgcccacgccaagccccacaccagtgagaagcatcagcaccgtgaacttgtctgagaatagcagtgttgtcatccccccacccgactacttggaatgcttgtccatgggggcagctgccgacaggagagcagattcggccaggacgacatccacctttaaggccccagcgtccaagcccgagaccgcggctcctaacgatgccaacgggactgcaaagccgccttttctcagcggagaaaacccctttgccactgtgaaactccgcccgactgtgacgaatgatcgctcggcacccatcattcgatga

SEQ ID NO: 60 - DNA, 4989 bp, Artificial Sequence
  Description of Artificial Sequence Synthetic polynucleotide
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtag

SEQ ID NO: 61 - DNA, 5109 bp, Artificial Sequence
  Description of Artificial Sequence Synthetic polynucleotide
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggagagtagatctggagaaaccaacagctgtgttgaagaaataatccgggagatgacctggcttccaccactttctgctattcaagcacctggcaaagtggaaccaaccaaatttccatttccaaataaggactctcagcttgtatcctctggacacaataatccaaagaaaggtgatgcagagccagagagtccagacagtggcacatcgaatacatcaatgctggaagatgaccttaagctaagcagtgatgaagaggagaatgaacagcaggcagctcagagaacggctctccgcgctctctctgacagcgccgtggtccagcagcccaactgcagaacctcggtgccttccagcaagggcagcagcagcagcagcagcagcggcagcagcagctcctccagcgactcagagagcagctccggatctgactcggagaccgagagcagctccagcgagagtgagggcagcaagcccccccacttctccagccccgaggctgaaccggcatcctctaacaagtggcagctggataaatggctaaacaaagttaatccccacaagcctcctattctgatccaaaatgaaagccacgggtcagagagcaatcagtactacaacccggtgaaagaggacgtccaggactgtgggaaagtccccgacgtttgccagcccagcctgagagagaaggagatcaagagcacttgcaaggaggagcaaaggccaaggacagccaacaaggcccctgggagtaaaggcgtgaagcagaagtccccgcccgcggccgtggccgtggcggtgagcgcagccgccccgccacccgcagtgccctgtgcgcccgcggagaacgcgcccgcgcctgcccggaggtccgcgggcaagaagcccaccaggcgcaccgagaggacctcagccggggacggcgccaactgccaccggcccgaggagcccgcggccgcggacgcgctggggacgagcgtggtggtccccccggagcccaccaaaaccaggccctgtggcaacaacagagcgagccaccgcaaggagctgcgctcctccgtgacctgcgagaagcgccgcacgcgggggctaagcaggatcgtccccaaatccaaggagttcattgagacagagtcgtcatcttcatcctcctcctcggactccgacctggagtccgagcaggaggagtaccctctgtccaaagcacagaccgtggctgcctctgcctcctccgggaatgatcagaggctgaaggaggccgctgccaacgggggcagtggtcctagggcccctgtaggctccatcaacgccaggaccaccagtgacatcgccaaggagctggaggagcagttctacacactggtcccctttggccggaacgaacttctctcccctctaaaggacagtgatgagatcaggtctctctgggtcaaaatcgacctgaccctcctgtccaggatcccagaacacctgccccaggagccaggggtattgagcgcccctgccaccaaggactctgagagcgcaccgcccagccacacctcggacacacctgcagaaaaggctttgccaaaatccaagaggaaacgcaagtgtgacaacgaagacgactacagggagatcaagaagtcccagggagagaaagacagctcttcaagactggccacctccaccagtaatactttgtctgcaaaccactgcaacatgaacatcaacagtgtggcaataccaataaataaaaatgaaaaaatgcttcggtcgcccatctcacccctctctgatgcatctaaacacaaatacaccagcgaggacttaacttcttccagccgacctaatggcaacagtttgtttacttcagcctcttccagcaaaaagcctaaggccgacagccagctgcagcctcacggcggagacctcacgaaagcagctcacaacaattctgaaaacattcccctccacaagtcacggccgcagacgaagccgtggtctccaggctccaacggccacagggactgcaagaggcagaaacttgtcttcgatgatatgcctcgcagtgccgattattttatgcaagaagctaaacgaatgaagcataaagcagatgcaatggtggaaaagtttggaaaggctttgaactatgctgaagcagcattgtcgtttatcgagtgtggaaatgcaatggaacaaggccccatggaatccaaatctccttatacgatgtattcagaaacagtagagctcatcaggtatgctatgagactaaaaacccactcaggccccaatgccacaccagaagacaaacaactggctgcattatgttaccgatgcctggccctcctgtactggcggatgtttcgactcaaaagggaccacgctgtaaagtattcaaaagcactaatcgactatttcaagaactcatctaaagccgcccaagccccatctccgtggggggccagtggaaagagcactggaaccccatcccccatgtctcccaacccctctcccgccagctccgtggggtctcagggcagcctctccaacgccagcgccctgtccccgtcgaccatcgtcagcatcccacagcgcatccaccagatggcggccaaccacgtcagcatcaccaacagcatcctgcacagctacgactactgggagatggccgacaacctggccaaggaaaaccgagaattcttcaacgacctggatctgctcatggggccggtcaccctgcacagcagcatggagcacctggtccagtactcccaacagggcctgcactggctgcggaacagcgcccacctgtcatag

SEQ ID NO: 62 - DNA, 3213 bp, Artificial Sequence
  Description of Artificial Sequence Synthetic polynucleotide
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatggcagatgatcagggctgtattgaagagcagggggttgaggattcagcaaatgaagattcagtggatgctaagccagaccggtcctcgtttgtaccgtccctcttcagtaagaagaagaaaaatgtcaccatgcgatccatcaagaccacccgggaccgagtgcctacatatcagtacaacatgaattttgaaaagctgggcaaatgcatcataataaacaacaagaactttgataaagtgacaggtatgggcgttcgaaacggaacagacaaagatgccgaggcgctcttcaagtgcttccgaagcctgggttttgacgtgattgtctataatgactgctcttgtgccaagatgcaagatctgcttaaaaaagcttctgaagaggaccatacaaatgccgcctgcttcgcctgcatcctcttaagccatggagaagaaaatgtaatttatgggaaagatggtgtcacaccaataaaggatttgacagcccactttaggggggatagatgcaaaacccttttagagaaacccaaactcttcttcattcaggcttgccgagggaccgagcttgatgatggcatccaggccgactcggggcccatcaatgacacagatgctaatcctcgatacaagatcccagtggaagctgacttcctcttcgcctattccacggttccaggctattactcgtggaggagcccaggaagaggctcctggtttgtgcaagccctctgctccatcctggaggagcacggaaaagacctggaaatcatgcagatcctcaccagggtgaatgacagagttgccaggcactttgagtctcagtctgatgacccacacttccatgagaagaagcagatcccctgtgtggtctccatgctcaccaaggaactctacttcagtcaatag

SEQ ID NO: 63 - DNA, 3423 bp, Artificial Sequence
  Description of Artificial Sequence Synthetic polynucleotide
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagcaagccagggctgagcaggaagaagaattcattagtaacactttattcaagaaaattcaggctttgcagaaggagaaagaaacccttgctgtaaattatgagaaagaagaagaattcctcactaatgagctctccagaaaattgatgcagttgcagcatgagaaagccgaactagaacagcatcttgaacaagagcaggaatttcaggtcaacaaactgatgaagaaaattaaaaaactggagaatgacaccatttctaagcaacttacattagaacagttgagacgggagaagattgaccttgaaaatacattggaacaagaacaagaagcactagttaatcgcctctggaaaaggatggataagcttgaagctgaaaagcgaatcctgcaggaaaaattagaccagcccgtctctgctccaccatcgcctagagatatctccatggagattgattctccagaaaatatgatgcgtcacatcaggtttttaaagaatgaagtggaacggctgaagaagcaactgagagctgctcagttacagcattcagagaaaatggcacagtatctggaggaggaacgtcacatgagagaagagaacttgaggctccagaggaagctgcagagggagatggagagaagagaagccctctgtcgacagctctccgagagtgagtccagcttagaaatggacgacgaaaggtattttaatgagatgtctgcacaaggattaagacctcgcactgtgtccagcccgatcccttacacaccttctccgagttcaagcaggcctatatcacctggtctatcatatgcaagtcacacggttggtttcacgccaccaacttcactgactagagctggaatgtcttattacaattccccgggtcttcacgtgcagcacatgggaacatcccatggtatcacaaggccttcaccacggagaagcaacagtcctgacaaattcaaacggcccacgccgcctccatctcccaacacacagaccccagtccagccacctccgcctccacctccgccacccatgcagcccacggtcccctcagcagccacctcgcagcctactccttcgcaacattcggcgcacccctcctcccagccttaa

SEQ ID NO: 64 - DNA, 5229 bp, Artificial Sequence
  Description of Artificial Sequence Synthetic polynucleotide
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagacacaacttcgaaaccagctaattcatgagttgatgcaccctgtattgagtggagaactgcagcctcggtccatttcagtagaagggagctccctcttaataggcgcctctaactctttagtggcagatcacttacaaagatgtggctatgaatattcactttctgttttctttccagaaagtggtttggcaaaagaaaaggtatttactatgcaggatctattacaactcattaaaatcaaccctacttccagtctctacaaatcactggtttcaggatctgataaagaaaatcaaaaaggttttcttatgcattttttaaaagaattggcagaatatcatcaagctaaagagagttgtaatatggaaactcagacaagttcgacatttaacagagattctctggctgagaagcttcagcttattgatgatcagtttgcagatgcttaccctcagcgtatcaagttcgaatctttagaaataaagctaaatgagtataagagagaaatagaagagcaacttcgggcagaaatgtgtcaaaagttgaagttttttaaagataccgagatagcaaaaattaaaatggaagcaaaaaaaaagtatgaaaaggagttaaccatgttccagaatgattttgaaaaagcttgtcaagcaaaatctgaagctctcgttcttcgggaaaagagtacccttgaaagaattcacaagcaccaagagattgaaacaaaagaaatttatgctcaaaggcaacttttactaaaagatatggatttgctaagaggaagagaagcagagctgaagcaaagagttgaagcttttgaattgaaccagaagctccaggaagaaaaacataaaagcataactgaggcacttaggagacaggagcagaatataaagagttttgaggagacctatgaccgaaagctcaagaatgaacttctaaagtatcaacttgaactgaaggatgactacatcattagaactaatcgactgattgaagatgaaaggaagaataaagaaaaagctgttcatttgcaagaggagctcatagctattaattcaaaaaaggaggaactcaatcaatctgtaaatcgtgtgaaagaacttgagcttgaattagagtctgtcaaagcccagtctttggcaataacaaaacaaaaccatatgctgaatgaaaaggttaaagagatgagtgattattcactactaaaagaagagaaactggagcttctggcacaaaataaattacttaaacaacaactggaagagagtagaaatgaaaacctgcgtctcctaaaccgcctagctcagccggctcctgaacttgcagtctttcagaaagaactacggaaagccgaaaaggctatagtggttgagcatgaggagttcgaaagctgcaggcaagctctgcacaaacaactgcaagacgaaattgagcattctgcacagctgaaggcccagattctaggttacaaagcttctgtaaagagtttaactactcaggttgccgatttaaaattgcaactgaagcaaactcagacagccctagagaatgaagtgtactgcaatccaaagcagtctgtgatcgatcgttctgtcaatggattaataaatggcaatgtggtgccttgcaatggtgagataagtggggatttcttgaacaatccttttaaacaggaaaacgttctagcacgtatggttgcatcaaggatcacaaattatccaactgcatgggtggagggtagttcccctgattctgaccttgagtttgtagccaatactaaggcaagggtcaaagagcttcagcaagaggccgaacgcttggaaaaggctttcagaagttaccatcggagagtcattaaaaactctgccaaaagcccactagcagcaaagagcccaccatctctgcacttgctggaagccttcaaaaacattacttccagttccccggaaagacatatttttggagaggacagagttgtctctgagcagcctcaagtgggcacacttgaagaaaggaatgacgtcgtggaagcactgacaggcagtgcagcctcgaggctccgcgggggcacttcctccagacgcctctcttccacaccccttccaaaagcaaaaagaagcctcgaaagtgaaatgtatctggaaggtctgggcagatcacacattgcttcccccagtccttgtcctgacagaatgcccctaccatcacccactgagtctaggcacagcctctccatccctcctgtctccagccctccggagcagaaagtgggtctttatcgaagacaaactgaacttcaagacaaaagtgaattttcagatgtggacaagctagcttttaaggataatgaggagtttgaatcatcttttgaatctgcagggaacatgccaaggcagttggaaatgggcgggctttctcctgccggggatatgtctcatgtggacgctgctgcagctgctgtgcccctctcatatcagcacccaagtgtagatcagaaacaaattgaagaacaaaaggaagaagaaaaaatacgggaacagcaagtgaaagaacgaaggcagagagaagaaagaaggcagagtaacctacaagaagttttagaaagggaacgaagagaactagaaaaactgtatcaggaaaggaagatgattgaagaatcactgaagattaaaataaaaaaggaattagaaatggaaaatgaattagaaatgagtaatcaagaaataaaagacaaatctgctcacagtgaaaatcctttagagaaatacatgaaaatcatccagcaggagcaagaccaggagtcggcagataagagctcaaaaaagatggtccaagaaggctccctagtggacacgctgcaatctagtgacaaagtcgaaagtttaacaggcttttctcatgaagaactagacgactcttggtaa

SEQ ID NO: 65 - DNA, 18 bp, Artificial Sequence
  Description of Artificial Sequence Synthetic oligonucleotide
tccaccgacgtaaaggcg

SEQ ID NO: 66 - DNA, 18 bp, Artificial Sequence
  Description of Artificial Sequence Synthetic oligonucleotide
taccgtgacgtccaccga

SEQ ID NO: 67 - DNA, 18 bp, Artificial Sequence
  Description of Artificial Sequence Synthetic oligonucleotide
gccttctggcccaggtgc

SEQ ID NO: 68 - DNA, 18 bp, Artificial Sequence
  Description of Artificial Sequence Synthetic oligonucleotide
tccaccgacaatgttatg

SEQ ID NO: 69 - DNA, 18 bp, Artificial Sequence
  Description of Artificial Sequence Synthetic oligonucleotide
accaatgaggagagtaga

SEQ ID NO: 70 - DNA, 18 bp, Artificial Sequence
  Description of Artificial Sequence Synthetic oligonucleotide
accaatgagatcatggag

SEQ ID NO: 71 - DNA, 16 bp, Artificial Sequence
  Description of Artificial Sequence Synthetic oligonucleotide
accaatgagatggcag

SEQ ID NO: 72 - DNA, 18 bp, Artificial Sequence
  Description of Artificial Sequence Synthetic oligonucleotide
accaatgagcaagccagg

SEQ ID NO: 73 - DNA, 18 bp, Artificial Sequence
  Description of Artificial Sequence Synthetic oligonucleotide
accaatgagacacaactt

(19) United States
(12) Patent Application Publication
(10) Pub. No.: US 2020/0208224 A1
(43) Pub. Date: Jul. 2, 2020

(54) Use Of FGFR Mutant Gene Panels In Identifying Cancer Patients That Will Be Responsive To Treatment With An FGFR Inhibitor

(71) Applicant: Janssen Pharmaceutica NV, Beerse (BE)

(72) Inventors: Suso Jesus Platero, Washington Crossing, PA (US); Jayaprakash Karkera, Germantown, MD (US)

(21) Appl. No.: 16/723,975

(22) Filed: Dec. 20, 2019

Related U.S. Application Data
(63) Continuation-in-part of application No. 16/136,201, filed on Sep. 19, 2018.
Application No. 16/136,201 is a continuation of application No. 14/858,627, filed on Sep. 18, 2015, now abandoned.
(60) Provisional application No. 62/056,159, filed on Sep. 26, 2014.

(51) Int. Cl. C12Q 1/6886

(52) U.S. Cl. CPC C12Q 1/6886; C12Q 2600/156; C12Q 2600/106

(57) ABSTRACT

Disclosed herein are methods of identifying a cancer patient that will be responsive to treatment with a fibroblast growth factor receptor (FGFR) inhibitor and methods of treating cancer patients. The methods involve evaluating a biological sample from the patient for the presence of one or more FGFR mutants from a FGFR mutant gene panel. Kits and primers for identifying the presence of one or more FGFR mutant genes in a biological sample are also disclosed herein.

### RELATED APPLICATIONS

This application is a continuation-in-part of U.S. application Ser. No. 16/136,201, filed Sep. 19, 2018, which is a continuation of U.S. application Ser. No. 14/858,627, filed Sep. 18, 2015, which claims the benefit of U.S. Provisional Application No. 62/056,159, filed on Sep. 26, 2014.

The entire teachings of the above applications are incorporated herein by reference.

### SEQUENCE LISTING

The instant application contains a Sequence Listing which has been submitted electronically in ASCII format and is hereby incorporated by reference in its entirety. Said ASCII copy, created on Dec. 20, 2019, is named 01482032042_Sequence_Listing_.txt and is 64 KB in size.

### TECHNICAL FIELD

Provided herein are methods of identifying a cancer patient that will be responsive to treatment with a fibroblast growth factor receptor inhibitor and methods of treating the same.

### BACKGROUND

The identification of genetic abnormalities can be useful in selecting the appropriate therapeutic(s) for cancer patients. This is also useful for cancer patients failing the main therapeutic option (front-line therapy) for that cancer type, particularly if there is no accepted standard of care for second and subsequent-line therapy. Fibroblast growth factor receptors (FGFRs) are a family of receptor tyrosine kinases involved in regulating cell survival, proliferation, migration and differentiation. FGFR alterations have been observed in some cancers. To date, there are no approved therapies that are efficacious in patients with FGFR alterations.

### SUMMARY

Disclosed herein are methods of identifying a cancer patient that will be responsive to treatment with a fibroblast growth factor receptor (FGFR) inhibitor comprising: evaluating a biological sample from the patient for a FGFR mutant from a FGFR mutant gene panel, wherein the FGFR mutant is a FGFR fusion gene or a FGFR single nucleotide polymorphism, and wherein said evaluating comprises amplifying cDNA with a pair of primers that bind to and amplify one or more FGFR mutants from the FGFR mutant gene panel; and determining whether the one or more FGFR mutants from the gene panel are present in the sample, wherein the presence of the one or more FGFR mutants indicates that the patient will be responsive to treatment with the FGFR inhibitor.

Also disclosed are methods of treating cancer in a patient comprising: evaluating a biological sample from the patient for the presence of one or more FGFR mutants from a FGFR mutant gene panel; and treating the patient with an FGFR inhibitor if one or more FGFR mutants are present in the sample.

Kits and primers for identifying the presence of one or more FGFR mutant genes in a biological sample are further provided herein.

### BRIEF DESCRIPTION OF THE DRAWINGS

The summary, as well as the following detailed description, is further understood when read in conjunction with the appended drawings. For the purpose of illustrating the disclosed methods, kits, and primers, there are shown in the drawings exemplary embodiments of the methods, kits, and primers; however, the methods, kits, and primers are not limited to the specific embodiments disclosed. In the drawings:

FIG. 1 is an illustration of exemplary FGFR fusion genes, the presence of at least one of which indicates that a patient will be responsive to treatment with an FGFR inhibitor. Also illustrated (small arrows) are exemplary primer locations for amplifying the fusion genes.

FIGS. 2A-2I represent Sanger sequencing results from FFPET samples positive for: FIG. 2A) FGFR3:TACC3 v1; FIG. 2B) FGFR3:TACC3 v3; FIG. 2C) FGFR3:TACC3 Intron; FIG. 2D) FGFR3:BAIAP2L1; FIG. 2E) FGFR2:AFF3; FIG. 2F) FGFR2:BICC1; FIG. 2G) FGFR2:CASP7; FIG. 2H) FGFR2:CCDC6; and FIG. 2I) FGFR2:OFD1.

FIG. 3 illustrates an exemplary strategy for SNP-specific qRT-PCR using a 3′ dideoxy wild type (WT) blocker oligonucleotide.

FIG. 4 illustrates an exemplary analytical validation strategy for detecting FGFR SNPs. Experiments were performed on engineered RK3E cell lines expressing the FGFR fusions and diluted into a wild type cell line harboring no FGFR3/FGFR2 fusions.

FIGS. 5A-5D illustrate SNP-specific PCR with dideoxy WT blocker for (FIG. 5A) G370C, (FIG. 5B) Y373C, (FIG. 5C) S249C, and (FIG. 5D) R248C.

FIGS. 6A-6I represent efficiency standard curves for the FGFR fusion gene assays: FIG. 6A) FGFR3:TACC3 v1; FIG. 6B) FGFR3:TACC3 v3; FIG. 6C) FGFR3:TACC3 Intron; FIG. 6D) FGFR3:BAIAP2L1; FIG. 6E) FGFR2:AFF3; FIG. 6F) FGFR2:BICC1; FIG. 6G) FGFR2:CASP7; FIG. 6H) FGFR2:CCDC6; and FIG. 6I) FGFR2:OFD1.

FIG. 7 is an exemplary representation of FGFR fusion gene status in bladder (primary and metastatic), NSCLC (adenocarcinoma and squamous), ovarian, esophageal (primary and metastatic), head and neck (H&N; primary and metastatic), endometrial (metastatic), breast, and prostate cancer.

FIG. 8 is an exemplary representation of FGFR fusion gene and mutation status in NSCLC adenocarcinoma and squamous cell carcinoma.

FIGS. 9A-9D represent exemplary results from phase I patient samples. Assays were performed using synthetic template assay control (ST), primers for GAPDH (quality control sample), or primers specific for: FIG. 9A) FGFR2:BICC1 fusions; FIG. 9B) FGFR3:TACC3 (exon 18:exon 1) fusions; FIG. 9C) FGFR2:CCDC6 fusions; or FIG. 9D) FGFR3:TACC3 v1, FGFR3:TACC3 v3, or FGFR2:CCDC6 fusions. Patient samples are as follows: FIG. 9A—urothelial carcinoma; FIG. 9B—bladder cancer; FIG. 9C—cholangiocarcinoma; and FIG. 9D—adrenal carcinoma.

FIG. 10 represents an exemplary Phase I Study design for a First-In-Human Study of JNJ-42756493 in patients with advanced solid tumor.

FIG. 11 represents the maximal inhibitory percentage reduction of sum of the diameters of targeted lesions from baseline with dose level greater than or equal to 6 mg. Solid tumor patients were treated with the FGFR inhibitor JNJ-42756493 at different doses administered either as a daily regimen or as an intermittent dosing regimen (7 days on −7 days off). Doses and tumor types are indicated. Reduction in tumor was measured as per the RECIST criteria. Patients whose tumors harbor FGFR gene translocations and mutations appear to be more sensitive to the FGFR inhibitor JNJ-42756493.

FIG. 12 illustrates the expression of various FGFR fusions in RK3E cells stably transfected with the indicated FGFR fusion.

FIGS. 13A-13B illustrate colony formation assays in RK3E cells stably transfected with the indicated FGFR fusion. (FIG. 13A) 0.1% cresyl crystal violet stained 6-well chambers and (FIG. 13B) bar graph illustrating the number of colonies/100 cells plated. Results are representative of two independent experiments.

FIGS. 14A-14H illustrate the expression of exemplary downstream targets in RK3E cells stably transfected with the indicated FGFR fusion.

### DETAILED DESCRIPTION OF ILLUSTRATIVE EMBODIMENTS

The disclosed methods, kits, and primers may be understood more readily by reference to the following detailed description taken in connection with the accompanying figures, which form a part of this disclosure. It is to be understood that the disclosed methods, kits, and primers are not limited to the specific methods, kits, and primers described and/or shown herein, and that the terminology used herein is for the purpose of describing particular embodiments by way of example only and is not intended to be limiting of the claimed methods, kits, and primers.

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed methods, kits, and primers which are, for clarity, described herein in the context of separate embodiments, may also be provided in combination in a single embodiment. Conversely, various features of the disclosed methods, kits, and primers that are, for brevity, described in the context of a single embodiment, may also be provided separately or in any subcombination.

As used herein, the singular forms “a,” “an,” and “the” include the plural.

The following abbreviations are used throughout the specification: FGFR (fibroblast growth factor receptor); LLOQ (lower limit of quantitation); FGFR3:TACC3 (fusion between genes encoding FGFR3 and transforming acidic coiled-coil containing protein 3); FGFR3:BAIAP2L1 (fusion between genes encoding FGFR3 and brain-specific angiogenesis inhibitor 1-associated protein 2-like protein 1); FGFR2:AFF3 (fusion between genes encoding FGFR2 and AF4/FMR2 family, member 3); FGFR2:BICC1 (fusion between genes encoding FGFR2 and bicaudal C homolog 1); FGFR2: CASP7 (fusion between genes encoding FGFR2 and caspase 7); FGFR2:CCDC6 (fusion between genes encoding FGFR2 and coiled-coil domain containing 6); FGFR2:OFD1 (fusion between genes encoding FGFR2 and oral-facial-digital syndrome 1); FFPET (Formalin-Fixed Paraffin-Embedded Tissue); SNP (single nucleotide polymorphism); NSCLC (Non-small-cell lung cancer), ct (cycle threshold).

As used herein, “treating” and like terms refer to reducing the severity and/or frequency of cancer symptoms, eliminating cancer symptoms and/or the underlying cause of said symptoms, reducing the frequency or likelihood of cancer symptoms and/or their underlying cause, and improving or remediating damage caused, directly or indirectly, by cancer.

“Biological samples” refers to any sample from a patient in which cancerous cells can be obtained and RNA can be isolated. Suitable biological samples include, but are not limited to, blood, lymph fluid, bone marrow, a solid tumor sample, or any combination thereof. In some embodiments, the biological sample can be FFPET.

As used herein, “pre-amplification” refers to a PCR procedure that is performed prior to the amplifying step in order to increase the quantity of template cDNA for the amplification step. A pre-amplification step can be performed, for example, using the TaqMan® PreAmp Master Mix (Life Technologies/Applied Biosystems® product #4391128).

As used herein, “amplifying,” “amplify,” and like terms refer to the generation of numerous identical copies of a nucleic acid sample. Suitable techniques for amplifying a nucleic acid sample include, but are not limited to, polymerase chain reaction (PCR) and real-time polymerase chain reaction (RT-PCR). In some embodiments, the amplifying step comprises RT-PCR.

### FGFR Mutants

As used herein, the phrase “FGFR mutant” refers to a FGFR fusion gene, a FGFR single nucleotide polymorphism, or both.

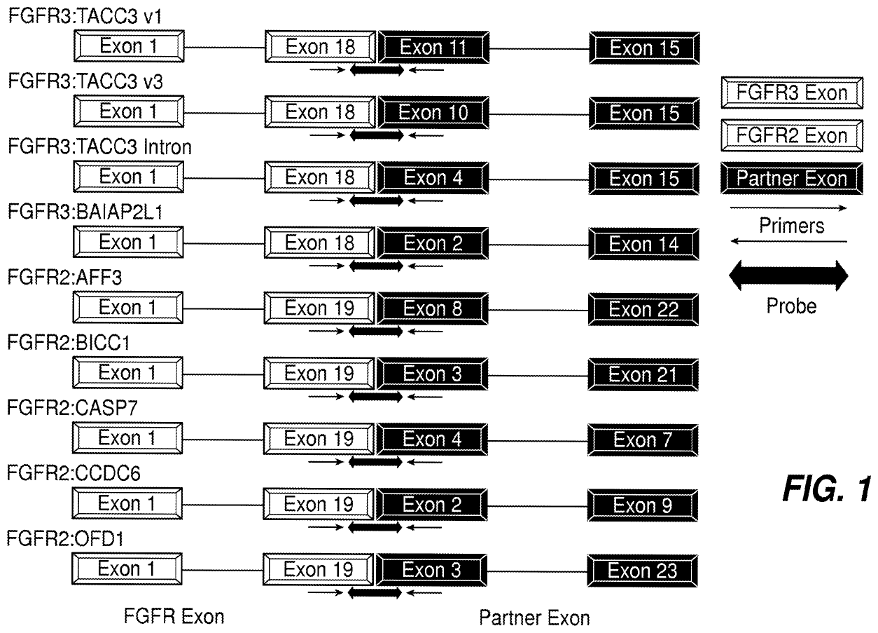

“FGFR fusion” or “FGFR fusion gene” refers to a gene encoding FGFR (e.g., FGRF2 or FGFR3), or a portion thereof, and one of the herein disclosed fusion partners, or portion thereof, created by a translocation between the two genes. The presence of one or more of the following FGFR fusion genes in a biological sample from a patient can be determined using the disclosed methods: FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCDC6, FGFR2:OFD1, or any combination thereof. Table 1 provides the FGFR fusion genes and the FGFR and fusion partner exons that are fused. FIG. 1 provides an illustration of the various FGFR fusion genes. The sequences of the individual FGFR fusion genes are disclosed in Table 16.

TABLE 1

| Fusion Gene | FGFR Exon | Partner Exon |
|---|---|---|
| FGFR3:TACC3 v1 | 18 | 11 |
| FCFR3:TACC3 v3 | 18 | 10 |
| FGFR3:TACC3 Intron | 18 | 4 |
| FGFR3:BAIAP2L1 | 18 | 2 |
| FGFR2:AFF3 | 19 | 8 |
| FGFR2:BICC1 | 19 | 3 |
| FGFR2:CASP7 | 19 | 4 |
| FGFR2:CCDC6 | 19 | 2 |
| FGFR2:OFD1 | 19 | 3 |

“FGFR single nucleotide polymorphism” (SNP) refers to a FGFR2 or FGFR3 gene in which a single nucleotide differs among individuals. In particular, FGFR single nucleotide polymorphism” (SNP) refers to a FGFR3 gene in which a single nucleotide differs among individuals. The presence of one or more of the following FGFR SNPs in a biological sample from a patient can be determined using the disclosed methods: FGFR3 R248C, FGFR3 S249C, FGFR3 G370C, FGFR3 Y373C, or any combination thereof. The sequences of the FGFR SNPs are provided in Table 2.

TABLE 2

| FGFR3 | |
|---|---|
| mutant | Sequence |
| FGFR3 | TCGGACCGCGGCAACTACACCTGCGTCGTGGAGAACAAGTTT |
| R248C | GGCAGCATCCGGCAGACGTACACGCTGGACGTGCTGGAG(T) |
| | GCTCCCCGCACCGGCCCATCCTGCAGGCGGGGCTGCCGGCCA |
| | ACCAGACGGCGGTGCTGGGCAGCGACGTGGAGTTCCACTGCA |
| | AGGTGTACAGTGACGCACAGCCCCACATCCAGTGGCTCAAGC |
| | ACGTGGAGGTGAATGGCAGCAAGGTGGGCCCGGACGGCACAC |
| | CCTACGTTACCGTGCTCA |
| | (SEQ ID NO: 1) |
| FGFR3 | GACCGCGGCAACTACACCTGCGTCGTGGAGAACAAGTTTGGC |
| S249C | AGCATCCGGCAGACGTACACGCTGGACGTGCTGGGTGAGGGC |
| | CCTGGGGCGGCGCGGGGGTGGGGGCGGCAGTGGCGGTGGTGG |
| | TGAGGGAGGGGGTGGCCCCTGAGCGTCATCTGCCCCCACAGA |
| | GCGCT(G)CCCGCACCGGCCCATCCTGCAGGCGGGGCTGCCG |
| | GCCAACCAGACGGCGGTGCTGGGCAGCGACGTGGAGTTCCAC |
| | TGCAAGGTGTACAGTGACGCACAGCCCCACATCCAGTGGCTC |
| | AAGCACGTGGAGGTGAATGGCAGCAAGGTGGGCCCGGACGGC |
| | ACACCCTACGTTACCGTGCTCAAGGTGGGCCACCGTGTGCAC |
| | GT |
| | (SEQ ID NO: 2) |
| FGFR3 | GCGGGCAATTCTATTGGGTTTTCTCATCACTCTGCGTGGCTG |
| G370C | GTGGTGCTGCCAGCCGAGGAGGAGCTGGTGGAGGCTGACGAG |
| | GCG(T)GCAGTGTGTATGCAGGCATCCTCAGCTACGGGGTGG |
| | GCTTCTTCCTGTTCATCCTGGTGGTGGCGGCTGTGACGCTCT |
| | GCCGCCTGCGCAGCCCCCCCAAGAAAGGCCTGGGCTCCCCCA |
| | CCGTGCACAAGATCTCCCGCTTCCCG |
| | (SEQ ID NO: 3) |
| FGFR3 | CTAGAGGTTCTCTCCTTGCACAACGTCACCTTTGAGGACGCC |
| Y373C* | GGGGAGTACACCTGCCTGGCGGGCAATTCTATTGGGTTTTCT |
| | CATCACTCTGCGTGGCTGGTGGTGCTGCCAGCCGAGGAGGAG |
| | CTGGTGGAGGCTGACGAGGCGGGCAGTGTGT(G)TGCAGGCA |
| | TCCTCAGCTACGGGGTGGGCTTCTTCCTGTTCATCCTGGTGG |
| | TGGCGGCTGTGACGCTCTGCCGCCTGCGCAGCCCCCCCAAGA |
| | AAGGCCTGGGCTCCCCCACCGTGCACAAGATCTCCCGCTTCC |
| | CGCTCAAGC |
| | (SEQ ID NO: 4) |

Sequences correspond to nucleotides 920-1510 of FGFR3 (Genebank ID # NM_000142.4).
Nucleotides in bold underline represent the SNP.
*Sometimes mistakenly referred to as Y375C in the literature.

As used herein, “FGFR mutant gene panel” includes one or more of the above listed FGFR mutants. In some embodiments, the FGFR mutant gene panel is dependent upon the patient's cancer type.

The FGFR mutant panel that is used in the evaluating step of the disclosed methods is based, in part, on the patient's cancer type. For patients with bladder cancer, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof.

For patients with metastatic bladder cancer, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof.

For patients with ovarian cancer, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof.

For patients with head and neck cancer, a suitable FGFR mutant gene panel can comprise FGFR3:BAIAP2L1, FGFR2:CASP7, FGFR3 R248C, FGFR3 S249C, FGFR3 G370C, or FGFR3 Y373C, or any combination thereof.

For patients with metastatic head and neck cancer, a suitable FGFR mutant gene panel can comprise FGFR3:BAIAP2L1, FGFR2:CASP7, or FGFR2:OFD1, or any combination thereof.

For patients with esophageal cancer, a suitable FGFR mutant gene panel can comprise FGFR3:TACC3 v1, FGFR3:TACC3 v3, FGFR2:BICC1, FGFR2:CASP7, FGFR3 R248C, FGFR3 S249C, FGFR3 G370C, or FGFR3 Y373C, or any combination thereof.

For patients with metastatic esophageal cancer, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCD6, or FGFR2:OFD1, or any combination thereof.

For patients with non-small-cell lung adenocarcinoma, a suitable FGFR mutant gene panel can comprise FGFR3:TACC3 v1, FGFR3:TACC3 v3, FGFR3:TACC3 Intron, FGFR3:BAIAP2L1, FGFR2:AFF3, FGFR2:CASP7, FGFR3 R248C, FGFR3 S249C, FGFR3 G370C, or FGFR3 Y373C, or any combination thereof.

For patients with non-small cell lung squamous cell carcinoma, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR2:CCDC6, FGFR3 R248C, FGFR3 S249C, FGFR3 G370C, or FGFR3 Y373C, or any combination thereof.

For patients with metastatic endometrial cancer, a suitable FGFR mutant gene panel can comprise FGFR3:TACC3 v1, FGFR3:TACC3 v3, FGFR3:TACC3 Intron, FGFR3:BAIAP2L1, FGFR2:CASP7, FGFR2:CCDC6, or FGFR2:OFD1, or any combination thereof.

For patients with breast cancer, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCD6, or FGFR2:OFD1, or any combination thereof.

### Primers for Amplifying FGFR Mutants

One skilled in the art knows that amplification of nucleic acid requires primers that are complementary, and bind to, a 5′ and 3′ region of the nucleic acid strand that flanks the region sought to be amplified. As used herein, “pair of primers” refers to the forward and reverse primers used in an amplifying step. Pairs of primers suitable for performing the disclosed methods are listed in Table 3.

TABLE 3

| Target | Forward Primer | Reverse Primer 5′-3′ |
|---|---|---|
| FGFR3:TACC3 V1 | GACCTGGACCGTGTCCTTACC | CTTCCCCAGTTCCAGGTTCTT |
| | (SEQ ID NO: 5) | (SEQ ID NO: 6) |
| FGFR3:TACC3 V3 | AGGACCTGGACCGTGTCCTT | TATAGGTCCGGTGGACAGGG |
| | (SEQ ID NO: 7) | (SEQ ID NO: 8) |
| FGFR3:TACC3 | GGCCATCCTGCCCCC | GAGCAGTCCAGGTCAGCCAG |
| Intron | (SEQ ID NO: 9) | (SEQ ID NO: 10) |
| FGFR3:BAIAP2L1 | CTGGACCGTGTCCTTACCGT | GCAGCCCAGGATTGAACTGT |
| | (SEQ ID NO: 11) | (SEQ ID NO: 12) |
| FGFR2:BICC1 | TGGATCGAATTCTCACTCTCACA | GCCAAGCAATCTGCGTATTTG |
| | (SEQ ID NO: 13) | (SEQ ID NO: 14) |
| FGFR2:AFF3 | TGGTAGAAGACTTGGATCGAATTCT | TCTCCCGGATTATTTCTTCAACA |
| | (SEQ ID NO: 15) | (SEQ ID NO: 16) |
| FGFR2:CASP7 | GCTCTTCAATACAGCCCTGATCA | ACTTGGATCGAATTCTCACTCTCA |
| | (SEQ ID NO: 17) | (SEQ ID NO: 18) |
| FGFR2:CCDC6 | TGGATCGAATTCTCACTCTCACA | GCAAAGCCTGAATTTTCTTGAATAA |
| | (SEQ ID NO: 19) | (SEQ ID NO: 20) |
| FGFR2:OFD1 | AGGGTGCATCAACTCATGAATTAG | ACTTGGATCGAATTCTCACTCTCA |
| | (SEQ ID NO: 21) | (SEQ ID NO: 22) |
| FGFR3 R248C | GCATCCGGCAGACGTACA | CCCCGCCTGCAGGAT |
| | (SEQ ID NO: 23) | (SEQ ID NO: 24) |
| FGFR3 S249C | GCATCCGGCAGACGTACA | CCCCGCCTGCAGGAT |
| | (SEQ ID NO: 25) | (SEQ ID NO: 26) |
| FGFR3 G370C | AGGAGCTGGTGGAGGCTGA | CCGTAGCTGAGGATGCCTG |
| | (SEQ ID NO: 27) | (SEQ ID NO: 28) |
| FGFR3 Y373C | CTGGTGGAGGCTGACGAG | AGCCCACCCCGTAGCT |
| | (SEQ ID NO: 29) | (SEQ ID NO: 30) |
| FGFR3 R248C | GTCGTGGAGAACAAGTTTGGC | GTCTGGTTGGCCGGCAG |
| | (SEQ ID NO: 31) | (SEQ ID NO: 32) |
| FGFR3 S249C | GTCGTGGAGAACAAGTTTGGC | GTCTGGTTGGCCGGCAG |
| | (SEQ ID NO: 33) | (SEQ ID NO: 34) |
| FGFR3 G370C | AGGAGCTGGTGGAGGCTGA | CCGTAGCTGAGGATGCCTG |
| | (SEQ ID NO: 35) | (SEQ ID NO: 36) |
| FGFR3 Y373C | GACGAGGCGGGCAGTG | GAAGAAGCCCACCCCGTAG |
| | (SEQ ID NO: 37) | (SEQ ID NO: 38) |

Disclosed herein are primers having the nucleic acid sequence of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or any combination thereof.

Also disclosed herein are sets of primers having the sequences of SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32, SEQ ID NO:33 and SEQ ID NO:34, SEQ ID NO:35 and SEQ ID NO:36, SEQ ID NO:37 and SEQ ID NO:38, or any combination thereof.

In some embodiments, the set of primers can have the sequence of SEQ ID NO:5 and SEQ ID NO:6. In some embodiments, the set of primers can have the sequence of SEQ ID NO:7 and SEQ ID NO:8. In some embodiments, the set of primers can have the sequence of SEQ ID NO:9 and SEQ ID NO:10. In some embodiments, the set of primers can have the sequence of SEQ ID NO:11 and SEQ ID NO:12. In some embodiments, the set of primers can have the sequence of SEQ ID NO:13 and SEQ ID NO:14. In some embodiments, the set of primers can have the sequence of SEQ ID NO:15 and SEQ ID NO:16. In some embodiments, the set of primers can have the sequence of SEQ ID NO:17 and SEQ ID NO:18. In some embodiments, the set of primers can have the sequence of SEQ ID NO:19 and SEQ ID NO:20. In some embodiments, the set of primers can have the sequence of SEQ ID NO:21 and SEQ ID NO:22. In some embodiments, the set of primers can have the sequence of SEQ ID NO:23 and SEQ ID NO:24. In some embodiments, the set of primers can have the sequence of SEQ ID NO:25 and SEQ ID NO:26. In some embodiments, the set of primers can have the sequence of SEQ ID NO:27 and SEQ ID NO:28. In some embodiments, the set of primers can have the sequence of SEQ ID NO:29 and SEQ ID NO:30. In some embodiments, the set of primers can have the sequence of SEQ ID NO:31 and SEQ ID NO:32. In some embodiments, the set of primers can have the sequence of SEQ ID NO:33 and SEQ ID NO:34. In some embodiments, the set of primers can have the sequence of SEQ ID NO:35 and SEQ ID NO:36. In some embodiments, the set of primers can have the sequence of SEQ ID NO:37 and SEQ ID NO:38. In some embodiments, the set of primers can have the sequences of any combination of the above sets of primers.

### FGFR Inhibitors for Use in the Disclosed Methods

Suitable FGFR inhibitors for use in the disclosed methods are provided herein.

In some embodiments, if one or more FGFR mutants are present in the sample, the patient can be treated with a FGFR inhibitor disclosed in U.S. Publ. No. 2013/0072457 A1 (incorporated herein by reference), including any tautomeric or stereochemically isomeric form thereof, and a N-oxide thereof, a pharmaceutically acceptable salt thereof, or a solvate thereof (suitable R groups are also disclosed in U.S. Publ. No. 2013/0072457 A1). In some aspects, for example, the patient can be treated with N-(3,5-dimethoxyphenyl)-N′-(1-methylethyl)-N-[3-(1-methyl-1H-pyrazol-4-yl)quinoxalin-6-yl]ethane-1,2-diamine (referred to herein as “JNJ-42756493” or “JNJ493”):

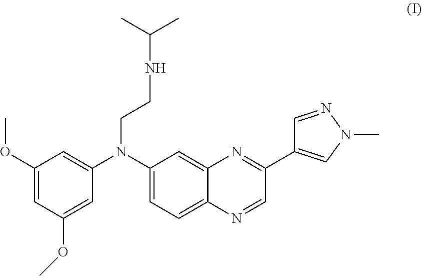 [SMILES: COc1cc(OC)cc(N(CCNC(C)C)c2ccc3ncc(-c4cnn(C)c4)nc3c2)c1]

including a N-oxide thereof, a pharmaceutically acceptable salt thereof, or a solvate thereof. In some aspects, the pharmaceutically acceptable salt is a HCl salt. In some aspects, the patient can be treated with JNJ493 base.

In some embodiments, the patient can be treated with a FGFR inhibitor if one or more FGFR mutants are present in the sample, wherein the FGFR inhibitor is N-[5-[2-(3,5-Dimethoxyphenyl)ethyl]-2H-pyrazol-3-yl]-4-(3,5-diemthylpiperazin-1-yl)benzamide (AZD4547), as described in Gavine, P. R., et al., AZD4547: An Orally Bioavailable, Potent, and Selective Inhibitor of the Fibroblast Growth Factor Receptor Tyrosine Kinase Family, Cancer Res. Apr. 15, 2012 72; 2045:

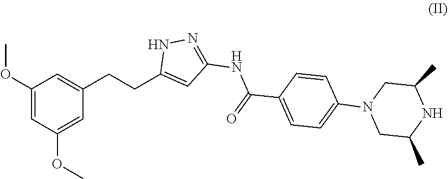 [SMILES: COc1cc(CCc2cc(NC(=O)c3ccc(N4C[C@@H](C)N[C@@H](C)C4)cc3)n[nH]2)cc(OC)c1]

including, when chemically possible, any tautomeric or stereochemically isomeric form thereof, and a N-oxide thereof, a pharmaceutically acceptable salt thereof, or a solvate thereof.

In some embodiments, the patient can be treated with a FGFR inhibitor if one or more FGFR mutants are present in the sample, wherein the FGFR inhibitor is 3-(2,6-Dichloro-3,5-dimethoxy-phenyl)-1-{6-[4-(4-ethyl-piperazin-1-yl)-phenylamino]-pyrimid-4-yl}-1-methyl-urea (NVP-BGJ398) as described in Int'l Publ. No. WO2006/000420:

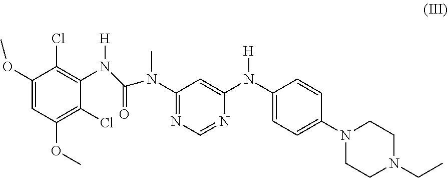 [SMILES: CCN1CCN(c2ccc(Nc3cc(N(C)C(=O)Nc4c(Cl)c(OC)cc(OC)c4Cl)ncn3)cc2)CC1]

including, when chemically possible, any tautomeric or stereochemically isomeric form thereof, and a N-oxide thereof, a pharmaceutically acceptable salt thereof, or a solvate thereof.

In some embodiments, the patient can be treated with a FGFR inhibitor if one or more FGFR mutants are present in the sample, wherein the FGFR inhibitor is 4-amino-5-fluoro-3-[6-(4-methylpiperazin-1-yl)-1H-benzimidazol-2-yl]-1H-quinolin-2-one (dovitinib) as described in Int't Publ. No. WO2006/127926:

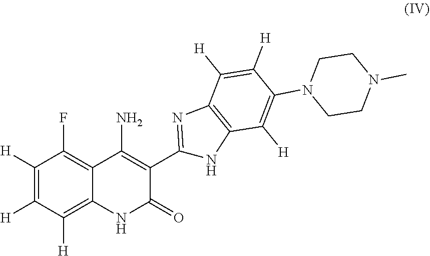 [SMILES: CN1CCN(c2ccc3nc(-c4c(N)c5c(F)cccc5[nH]c4=O)[nH]c3c2)CC1]

including, when chemically possible, any tautomeric or stereochemically isomeric form thereof, and a N-oxide thereof, a pharmaceutically acceptable salt thereof, or a solvate thereof.

In some embodiments, the patient can be treated with a FGFR inhibitor if one or more FGFR mutants are present in the sample, wherein the FGFR inhibitor is 6-(7-((1-Aminocyclopropyl)-methoxy)-6-methoxyquinolin-4-yloxy)-N-methyl-1-naphthamide (AL3810) (lucitanib; E-3810), as described in Bello, E. et al., E-3810 Is a Potent Dual Inhibitor of VEGFR and FGFR that Exerts Antitumor Activity in Multiple Preclinical Models, Cancer Res Feb. 15, 2011 71(A)1396-1405 and Int'l Publ. No. WO2008/112408:

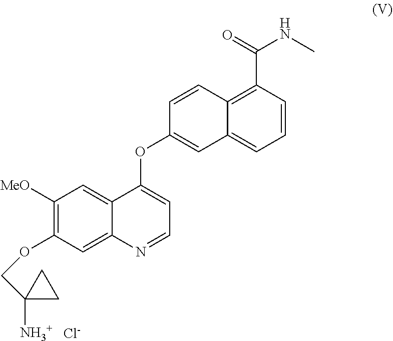 [SMILES: CNC(=O)c1cccc2cc(Oc3ccnc4cc(OCC5([NH3+])CC5)c(OC)cc34)ccc12 ; [Cl-]]

including, when chemically possible, any tautomeric or stereochemically isomeric form thereof, and a N-oxide thereof, a pharmaceutically acceptable salt thereof, or a solvate thereof.

In some embodiments, the patient can be treated with a FGFR inhibitor if one or more FGFR mutants are present in the sample, wherein the FGFR inhibitor is an anti-FGFR2 antibody such as that described in WO2013/076186.

Additional suitable FGFR inhibitors include BAY1163877 (Bayer), BAY1179470 (Bayer), TAS-120 (Taiho), ARQ087 (ArQule), ASP5878 (Astellas), FF284 (Chugai), FP-1039 (GSK/FivePrime), Blueprint, LY-2874455 (Lilly), RG-7444 (Roche), or any combination thereof, including, when chemically possible, any tautomeric or stereochemically isomeric forms thereof, N-oxides thereof, pharmaceutically acceptable salts thereof, or solvates thereof.

In some embodiments, the patient can be treated with a FGFR inhibitor if one or more FGFR mutants are present in the sample, wherein the FGFR inhibitor is BAY1163877 (Bayer), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is BAY1179470 (Bayer), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is TAS-120 (Taiho), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is ARQ087 (ArQule), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is ASP5878 (Astellas), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is FF284 (Chugai), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is FP-1039 (GSK/FivePrime), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is Blueprint, including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is LY-2874455 (Lilly), including, when chemically possible, any tautomeric or stereochemically isomeric form thereof, N-oxide thereof, pharmaceutically acceptable salt thereof, or solvate thereof.

In some embodiments, the patient can be treated with a FGFR inhibitor if one or more FGFR mutants are present in the sample, wherein the FGFR inhibitor is RG-7444 (Roche), including, when chemically possible, any tautomeric or stereochemically isomeric form thereof, N-oxide thereof, pharmaceutically acceptable salt thereof, or solvate thereof.

Salts can be synthesized from a parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002, which is incorporated herein by reference. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used. The FGFR inhibitors for use in the disclosed methods may exist as mono- or di-salts depending upon the pKa of the acid from which the salt is formed.

Acid addition salts may be formed with a wide variety of acids, both inorganic and organic. Examples of acid addition salts include salts formed with an acid including, but not limited to, acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecyl sulphuric, ethane-1,2-di sulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic, (±)-DL-lactic), lactobionic, maleic, malic, (−)-L-malic, malonic, (±)-DL-mandelic, methanesulphonic, naphthalenesulphonic (e.g. naphthalene-2-sulphonic), naphthalene-1,5-di sulphonic, 1-hydroxy-2-naphthoic, nicotinic, nitric, oleic, orotic, oxalic, palmitic, pamoic, phosphoric, propionic, L-pyroglutamic, pyruvic, salicylic, 4-amino-salicylic, sebacic, stearic, succinic, sulphuric, tannic, (+)-L-tartaric, thiocyanic, toluenesulphonic (e.g. p-toluenesulphonic), undecylenic and valeric acids, as well as acylated amino acids and cation exchange resins.

One particular group of salts consists of salts formed from acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, propanoic, butanoic, malonic, glucuronic and lactobionic acids. Another group of acid addition salts includes salts formed from acetic, adipic, ascorbic, aspartic, citric, DL-Lactic, fumaric, gluconic, glucuronic, hippuric, hydrochloric, glutamic, DL-malic, methanesulphonic, sebacic, stearic, succinic and tartaric acids.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺).

Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the disclosed compounds. Compounds containing an amine function may also form N-oxides. A reference herein to a compound that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. (1977), 7, 509-514) in which the amine compound is reacted with m-chloroperoxybenzoic acid (MCPBA), for example, in an inert solvent such as dichloromethane.

As used herein, the term “solvate” means a physical association of the compound with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include the disclosed compounds in combination with water, isopropanol, ethanol, methanol, DMSO, ethyl acetate, acetic acid, ethanolamine and the like. The compound may exert its biological effects while in solution.

Solvates are well known in pharmaceutical chemistry. They can be important to the processes for the preparation of a substance (e.g. in relation to their purification), the storage of the substance (e.g. its stability) and the ease of handling of the substance, and are often formed as part of the isolation or purification stages of a chemical synthesis. A person skilled in the art can determine by means of standard and long used techniques whether a hydrate or other solvate has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid State NMR (SS-NMR, also known as Magic Angle Spinning NMR or MAS-NMR). Such techniques are as much a part of the standard analytical toolkit of the skilled chemist as NMR, IR, HPLC and MS. Alternatively the skilled person can deliberately form a solvate using crystallisation conditions that include an amount of the solvent required for the particular solvate. Thereafter the standard methods described above, can be used to establish whether solvates had formed. Also encompassed are any complexes (e.g. inclusion complexes or clathrates with compounds such as cyclodextrins, or complexes with metals) of the FGFR inhibitor.

Furthermore, the compound may have one or more polymorph (crystalline) or amorphous forms.

The compounds include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. In one embodiment, the compounds contain no radioactive isotopes. Such compounds are preferred for therapeutic use. In another embodiment, however, the compound may contain one or more radioisotopes. Compounds containing such radioisotopes may be useful in a diagnostic context.

In some embodiments, the patient is treated with a FGFR inhibitor if one or more FGFR mutants are present in the sample, wherein the FGFR inhibitor is N-(3,5-dimethoxyphenyl)-N′-(1-methylethyl)-N-[3-(1-methyl-1H-pyrazol-4-yl)quinoxalin-6-yl]ethane-1,2-diamine (referred to herein “JNJ-42756493”), or a pharmaceutically acceptable salt thereof or a solvate thereof.

### Methods of Treating Cancer in a Patient

Disclosed herein are methods of treating cancer in a patient comprising: evaluating a biological sample from the patient for the presence of one or more FGFR mutants from a FGFR mutant gene panel; and treating the patient with an FGFR inhibitor if one or more FGFR mutants are present in the sample.

The disclosed methods can be used to treat a variety of cancer types including, but not limited to, bladder cancer, metastatic bladder cancer, ovarian cancer, head and neck cancer, metastatic head and neck cancer, esophageal cancer, metastatic esophageal cancer, non-small-cell lung adenocarcinoma, non-small cell lung squamous cell carcinoma, prostate cancer, lung cancer, gastric cancer, urothelial carcinoma, small cell lung cancer, breast cancer, endometrial cancer, metastatic endometrial cancer, cholangiocarcinoma, hepatocellular carcinoma, glioblastoma, gliomas, colon carcinoma, sarcomas, solid tumors of squamous origin, and multiple myeloma.

The FGFR mutant panel that is used in the evaluating step is based, in part, on the patient's cancer type. For patients with bladder cancer, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof. Accordingly, in some embodiments, a patient having bladder cancer is treated with an FGFR inhibitor if FGFR3:TACC3 v1 is present in the sample. In some embodiments, a patient having bladder cancer is treated with an FGFR inhibitor if FGFR3:TACC3 v3 is present in the sample. In some embodiments, a patient having bladder cancer is treated with an FGFR inhibitor if FGFR3:BAIAP2L1 is present in the sample. In some embodiments, a patient having bladder cancer is treated with an FGFR inhibitor if FGFR2:BICC1 is present in the sample. In some embodiments, a patient having bladder cancer is treated with an FGFR inhibitor if FGFR2:AFF3 is present in the sample. In some embodiments, a patient having bladder cancer is treated with an FGFR inhibitor if FGFR2:CASP7 is present in the sample. In some embodiments, a patient having bladder cancer is treated with an FGFR inhibitor if FGFR3 R248C is present in the sample. In some embodiments, a patient having bladder cancer is treated with an FGFR inhibitor if FGFR3 S249C is present in the sample. In some embodiments, a patient having bladder cancer is treated with an FGFR inhibitor if FGFR3 G370C is present in the sample. In some embodiments, a patient having bladder cancer is treated with an FGFR inhibitor if FGFR3 Y373C is present in the sample. In some embodiments, a patient having bladder cancer is treated with an FGFR inhibitor if any combination of the above FGFR mutants is present in the sample.

For patients with metastatic bladder cancer, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof. Accordingly, in some embodiments, a patient having metastatic bladder cancer is treated with an FGFR inhibitor if FGFR3:TACC3 v1 is present in the sample. In some embodiments, a patient having metastatic bladder cancer is treated with an FGFR inhibitor if FGFR3:TACC3 v3 is present in the sample. In some embodiments, a patient having metastatic bladder cancer is treated with an FGFR inhibitor if FGFR3:BAIAP2L1 is present in the sample. In some embodiments, a patient having metastatic bladder cancer is treated with an FGFR inhibitor if FGFR2:BICC1 is present in the sample. In some embodiments, a patient having metastatic bladder cancer is treated with an FGFR inhibitor if FGFR2:AFF3 is present in the sample. In some embodiments, a patient having metastatic bladder cancer is treated with an FGFR inhibitor if FGFR2:CASP7 is present in the sample. In some embodiments, a patient having metastatic bladder cancer is treated with an FGFR inhibitor if FGFR3 R248C is present in the sample. In some embodiments, a patient having metastatic bladder cancer is treated with an FGFR inhibitor if FGFR3 S249C is present in the sample. In some embodiments, a patient having metastatic bladder cancer is treated with an FGFR inhibitor if FGFR3 G370C is present in the sample. In some embodiments, a patient having metastatic bladder cancer is treated with an FGFR inhibitor if FGFR3 Y373C is present in the sample. In some embodiments, a patient having metastatic bladder cancer is treated with an FGFR inhibitor if any combination of the above FGFR mutants is present in the sample.

For patients with ovarian cancer, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof. Accordingly, in some embodiments, a patient having ovarian cancer is treated with an FGFR inhibitor if FGFR3:TACC3 v1 is present in the sample. In some embodiments, a patient having ovarian cancer is treated with an FGFR inhibitor if FGFR3:TACC3 v3 is present in the sample. In some embodiments, a patient having ovarian cancer is treated with an FGFR inhibitor if FGFR3:BAIAP2L1 is present in the sample. In some embodiments, a patient having ovarian cancer is treated with an FGFR inhibitor if FGFR2:BICC1 is present in the sample. In some embodiments, a patient having ovarian cancer is treated with an FGFR inhibitor if FGFR2:AFF3 is present in the sample. In some embodiments, a patient having ovarian cancer is treated with an FGFR inhibitor if FGFR2:CASP7 is present in the sample. In some embodiments, a patient having ovarian cancer is treated with an FGFR inhibitor if FGFR3 R248C is present in the sample. In some embodiments, a patient having ovarian cancer is treated with an FGFR inhibitor if FGFR3 S249C is present in the sample. In some embodiments, a patient having ovarian cancer is treated with an FGFR inhibitor if FGFR3 G370C is present in the sample. In some embodiments, a patient having ovarian cancer is treated with an FGFR inhibitor if FGFR3 Y373C is present in the sample. In some embodiments, a patient having ovarian cancer is treated with an FGFR inhibitor if any combination of the above FGFR mutants is present in the sample.

For patients with head and neck cancer, for example, a suitable FGFR mutant gene panel can comprise FGFR3:BAIAP2L1, FGFR2:CASP7, FGFR3 R248C, FGFR3 S249C, FGFR3 G370C, or FGFR3 Y373C, or any combination thereof. Accordingly, in some embodiments, a patient having head and neck cancer is treated with an FGFR inhibitor if FGFR3:BAIAP2L1 is present in the sample. In some embodiments, a patient having head and neck cancer is treated with an FGFR inhibitor if FGFR2:CASP7 is present in the sample. In some embodiments, a patient having head and neck cancer is treated with an FGFR inhibitor if FGFR3 R248C is present in the sample. In some embodiments, a patient having head and neck cancer is treated with an FGFR inhibitor if FGFR3 S249C is present in the sample. In some embodiments, a patient having head and neck cancer is treated with an FGFR inhibitor if FGFR3 G370C is present in the sample. In some embodiments, a patient having head and neck cancer is treated with an FGFR inhibitor if FGFR3 Y373C is present in the sample. In some embodiments, a patient having head and neck cancer is treated with an FGFR inhibitor if any combination of the above FGFR mutants is present in the sample.

For patients with metastatic head and neck cancer, for example, a suitable FGFR mutant gene panel can comprise FGFR3:BAIAP2L1, FGFR2:CASP7, or FGFR2:OFD1, or any combination thereof. Accordingly, in some embodiments, a patient having metastatic head and neck cancer is treated with an FGFR inhibitor if FGFR3:BAIAP2L1 is present in the sample. In some embodiments, a patient having metastatic head and neck cancer is treated with an FGFR inhibitor if FGFR2:CASP7 is present in the sample. In some embodiments, a patient having metastatic head and neck cancer is treated with an FGFR inhibitor if FGFR2:OFD1 is present in the sample. In some embodiments, a patient having metastic head and neck cancer is treated with an FGFR inhibitor if any combination of the above FGFR mutants is present in the sample.

For patients with esophageal cancer, for example, a suitable FGFR mutant gene panel can comprise FGFR3:TACC3 v1, FGFR3:TACC3 v3, FGFR2:BICC1, FGFR2:CASP7, FGFR3 R248C, FGFR3 S249C, FGFR3 G370C, or FGFR3 Y373C, or any combination thereof. Accordingly, in some embodiments, a patient having esophageal cancer is treated with an FGFR inhibitor if FGFR3:TACC3 v1 is present in the sample. In some embodiments, a patient having esophageal cancer is treated with an FGFR inhibitor if FGFR3:TACC3 v3 is present in the sample. In some embodiments, a patient having esophageal cancer is treated with an FGFR inhibitor if FGFR2:BICC1 is present in the sample. In some embodiments, a patient having esophageal cancer is treated with an FGFR inhibitor if FGFR2:CASP7 is present in the sample. In some embodiments, a patient having esophageal cancer is treated with an FGFR inhibitor if FGFR3 R248C is present in the sample. In some embodiments, a patient having esophageal cancer is treated with an FGFR inhibitor if FGFR3 S249C is present in the sample. In some embodiments, a patient having esophageal cancer is treated with an FGFR inhibitor if FGFR3 G370C is present in the sample. In some embodiments, a patient having esophageal cancer is treated with an FGFR inhibitor if FGFR3 Y373C is present in the sample. In some embodiments, a patient having esophageal cancer is treated with an FGFR inhibitor if any combination of the above FGFR mutants is present in the sample.

For patients with metastatic esophageal cancer, for example, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCD6, or FGFR2:OFD1, or any combination thereof. Accordingly, in some embodiments, a patient having metastatic esophageal cancer is treated with an FGFR inhibitor if FGFR3:TACC3 v1 is present in the sample. In some embodiments, a patient having metastatic esophageal cancer is treated with an FGFR inhibitor if FGFR3:TACC3 v3 is present in the sample. In some embodiments, a patient having metastatic esophageal cancer is treated with an FGFR inhibitor if FGFR3:TACC3 Intron is present in the sample. In some embodiments, a patient having metastatic esophageal cancer is treated with an FGFR inhibitor if FGFR3:BAIAP2L1 is present in the sample. In some embodiments, a patient having metastatic esophageal cancer is treated with an FGFR inhibitor if FGFR2:BICC1 is present in the sample. In some embodiments, a patient having metastatic esophageal cancer is treated with an FGFR inhibitor if FGFR2:AFF3 is present in the sample. In some embodiments, a patient having metastatic esophageal cancer is treated with an FGFR inhibitor if FGFR2:CASP7 is present in the sample. In some embodiments, a patient having metastatic esophageal cancer is treated with an FGFR inhibitor if FGFR2:CCD6 is present in the sample. In some embodiments, a patient having metastatic esophageal cancer is treated with an FGFR inhibitor if FGFR2:OFD1 is present in the sample. In some embodiments, a patient having metastatic esophageal cancer is treated with an FGFR inhibitor if any combination of the above FGFR mutants is present in the sample.

For patients with non-small cell lung (NSCL) adenocarcinoma, for example, a suitable FGFR mutant gene panel can comprise FGFR3:TACC3 v1, FGFR3:TACC3 v3, FGFR3:TACC3 Intron, FGFR3:BAIAP2L1, FGFR2:AFF3, FGFR2:CASP7, FGFR3 R248C, FGFR3 S249C, FGFR3 G370C, or FGFR3 Y373C, or any combination thereof. Accordingly, in some embodiments, a patient having NSCL adenocarcinoma is treated with an FGFR inhibitor if FGFR3:TACC3 v1 is present in the sample. In some embodiments, a patient having NSCL adenocarcinoma is treated with an FGFR inhibitor if FGFR3:TACC3 v3 is present in the sample. In some embodiments, a patient having NSCL adenocarcinoma is treated with an FGFR inhibitor if FGFR3:TACC3 Intron is present in the sample. In some embodiments, a patient having NSCL adenocarcinoma is treated with an FGFR inhibitor if FGFR3:BAIAP2L1 is present in the sample. In some embodiments, a patient having NSCL adenocarcinoma is treated with an FGFR inhibitor if FGFR2:AFF3 is present in the sample. In some embodiments, a patient having NSCL adenocarcinoma is treated with an FGFR inhibitor if FGFR2:CASP7 is present in the sample. In some embodiments, a patient having NSCL adenocarcinoma is treated with an FGFR inhibitor if FGFR3 R248C is present in the sample. In some embodiments, a patient having NSCL adenocarcinoma is treated with an FGFR inhibitor if FGFR3 S249C is present in the sample. In some embodiments, a patient having NSCL adenocarcinoma is treated with an FGFR inhibitor if FGFR3 G370C is present in the sample. In some embodiments, a patient having NSCL adenocarcinoma is treated with an FGFR inhibitor if FGFR3 Y373C is present in the sample. In some embodiments, a patient having NSCL adenocarcinoma is treated with an FGFR inhibitor if any combination of the above FGFR mutants is present in the sample.

For patients with non-small cell lung (NSCL) squamous cell carcinoma, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR2:CCDC6, FGFR3 R248C, FGFR3 S249C, FGFR3 G370C, or FGFR3 Y373C, or any combination thereof. Accordingly, in some embodiments, a patient having NSCL squamous cell carcinoma is treated with an FGFR inhibitor if FGFR3:TACC3 v1 is present in the sample. In some embodiments, a patient having NSCL squamous cell carcinoma is treated with an FGFR inhibitor if FGFR3:TACC3 v3 is present in the sample. In some embodiments, a patient having NSCL squamous cell carcinoma is treated with an FGFR inhibitor if FGFR3:BAIAP2L1 is present in the sample. In some embodiments, a patient having NSCL squamous cell carcinoma is treated with an FGFR inhibitor if FGFR2:BICC1 is present in the sample. In some embodiments, a patient having NSCL squamous cell carcinoma is treated with an FGFR inhibitor if FGFR2:AFF3 is present in the sample. In some embodiments, a patient having NSCL squamous cell carcinoma is treated with an FGFR inhibitor if FGFR2:CASP7 is present in the sample. In some embodiments, a patient having NSCL squamous cell carcinoma is treated with an FGFR inhibitor if FGFR2:CCDC6 is present in the sample. In some embodiments, a patient having NSCL squamous cell carcinoma is treated with an FGFR inhibitor if FGFR3 R248C is present in the sample. In some embodiments, a patient having NSCL squamous cell carcinoma is treated with an FGFR inhibitor if FGFR3 S249C is present in the sample. In some embodiments, a patient having NSCL squamous cell carcinoma is treated with an FGFR inhibitor if FGFR3 G370C is present in the sample. In some embodiments, a patient having NSCL squamous cell carcinoma is treated with an FGFR inhibitor if FGFR3 Y373C is present in the sample. In some embodiments, a patient having NSCL squamous cell carcinoma is treated with an FGFR inhibitor if any combination of the above FGFR mutants is present in the sample.

For patients with metastatic endometrial cancer, for example, a suitable FGFR mutant gene panel can comprise FGFR3:TACC3 v1, FGFR3:TACC3 v3, FGFR3:TACC3 Intron, FGFR3:BAIAP2L1, FGFR2:CASP7, FGFR2:CCDC6, or FGFR2:OFD1, or any combination thereof. Accordingly, in some embodiments, a patient having metastatic endometrial cancer is treated with an FGFR inhibitor if FGFR3:TACC3 v1 is present in the sample. In some embodiments, a patient having metastatic endometrial cancer is treated with an FGFR inhibitor if FGFR3:TACC3 v3 is present in the sample. in some embodiments, a patient having metastatic endometrial cancer is treated with an FGFR inhibitor if FGFR3:TACC3 Intron is present in the sample. In some embodiments, a patient having metastatic endometrial cancer is treated with an FGFR inhibitor if FGFR3:BAIAP2L1 is present in the sample. In some embodiments, a patient having metastatic endometrial cancer is treated with an FGFR inhibitor if FGFR2:CASP7 is present in the sample. In some embodiments, a patient having metastatic endometrial cancer is treated with an FGFR inhibitor if FGFR2:CCDC6 is present in the sample. In some embodiments, a patient having metastatic endometrial cancer is treated with an FGFR inhibitor if FGFR2:OFD1 is present in the sample. In some embodiments, a patient having metastatic endometrial cancer is treated with an FGFR inhibitor if any combination of the above FGFR mutants is present in the sample.

For patients with breast cancer, for example, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCD6, or FGFR2:OFD1, or any combination thereof. Accordingly, in some embodiments, a patient having breast cancer is treated with an FGFR inhibitor if FGFR3:TACC3 v1 is present in the sample. In some embodiments, a patient having breast cancer is treated with an FGFR inhibitor if FGFR3:TACC3 v3 is present in the sample. In some embodiments, a patient having breast cancer is treated with an FGFR inhibitor if FGFR3:TACC3 Intron is present in the sample. In some embodiments, a patient having breast cancer is treated with an FGFR inhibitor if FGFR3:BAIAP2L1 is present in the sample. In some embodiments, a patient having breast cancer is treated with an FGFR inhibitor if FGFR2:BICC1 is present in the sample. In some embodiments, a patient having breast cancer is treated with an FGFR inhibitor if FGFR2:AFF3 is present in the sample. In some embodiments, a patient having breast cancer is treated with an FGFR inhibitor if FGFR2:CASP7 is present in the sample. In some embodiments, a patient having breast cancer is treated with an FGFR inhibitor if FGFR2:CCD6 is present in the sample. In some embodiments, a patient having breast cancer is treated with an FGFR inhibitor if FGFR2:OFD1 is present in the sample. In some embodiments, a patient having breast cancer is treated with an FGFR inhibitor if any combination of the above FGFR mutants is present in the sample.

For patients with hepatocellular carcinoma, for example, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCDC6, FGFR2:OFD1, FGFR3 R248C, FGFR3 S249C, FGFR3 G370C, or FGFR3 Y373C, or any combination thereof. Accordingly, in some embodiments, a patient having hepatocellular carcinoma is treated with an FGFR inhibitor if FGFR3:TACC3 v1 is present in the sample. In some embodiments, a patient having hepatocellular carcinoma is treated with an FGFR inhibitor if FGFR3:TACC3 v3 is present in the sample. In some embodiments, a patient having hepatocellular carcinoma is treated with an FGFR inhibitor if FGFR3:TACC3 Intron is present in the sample. In some embodiments, a patient having hepatocellular carcinoma is treated with an FGFR inhibitor if FGFR3:BAIAP2L1 is present in the sample. In some embodiments, a patient having hepatocellular carcinoma is treated with an FGFR inhibitor if FGFR2:BICC1 is present in the sample. In some embodiments, a patient having hepatocellular carcinoma is treated with an FGFR inhibitor if FGFR2:AFF3 is present in the sample. In some embodiments, a patient having hepatocellular carcinoma is treated with an FGFR inhibitor if FGFR2:CASP7 is present in the sample. In some embodiments, a patient having hepatocellular carcinoma is treated with an FGFR inhibitor if FGFR2:CCDC6 is present in the sample. In some embodiments, a patient having hepatocellular carcinoma is treated with an FGFR inhibitor if FGFR2:OFD1 is present in the sample. In some embodiments, a patient having hepatocellular carcinoma is treated with an FGFR inhibitor if FGFR3 R248C is present in the sample. In some embodiments, a patient having hepatocellular carcinoma is treated with an FGFR inhibitor if FGFR3 S249C is present in the sample. In some embodiments, a patient having hepatocellular carcinoma is treated with an FGFR inhibitor if FGFR3 G370C is present in the sample. In some embodiments, a patient having hepatocellular carcinoma is treated with an FGFR inhibitor if FGFR3 Y373C is present in the sample. In some embodiments, a patient having hepatocellular carcinoma is treated with an FGFR inhibitor if any combination of the above FGFR mutants is present in the sample.

In some embodiments, the evaluating step comprises: isolating RNA from the biological sample; synthesizing cDNA from the isolated RNA; pre-amplifying the cDNA; and amplifying the pre-amplified cDNA with a pair of primers that bind to and amplify one or more FGFR mutants from the FGFR mutant gene panel.

Isolating RNA from the biological sample can be performed by a number of procedures known to one skilled in the art. In one embodiment, RNA can be isolated from the biological sample using an AllPrep DNA/RNA FFPE Kit from Qiagen (product #80234)

Synthesizing cDNA from isolated RNA can be performed by a number of procedures known to one skilled in the art. In one embodiment, cDNA can be synthesized from isolated RNA using a High Capacity cDNA Reverse Transcriptase Kit with RNase Inhibitor from ABI (product #4374966).

Pre-amplification of cDNA can be performed by a number of procedures known to one skilled in the art. Amplification procedures are well known in the art. In one embodiment, cDNA can be pre-amplified using a TaqMan® PreAmp Master Mix (Life Technologies/Applied Biosystems® product #4391128).

In some embodiments, the amplifying step can comprise performing real-time PCR (qRT-PCR). Exemplary qRT-PCR procedures are discussed in the Example section herein. In some aspects, the qRT-PCR can be a Taqman® Real-Time PCR assay. qRT-PCR procedures can involve the use of probes to increase the specificity of the assay. Suitable probes for use in the qRT-PCR assay include any of the probes disclosed herein, for example, the probes disclosed in Table 15. In some embodiments, for example, the real-time PCR can be performed with one or more probes comprising SEQ ID NO: 43, SEQ ID NO:44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and/or SEQ ID NO: 55. In other embodiments, the real-time PCR can be performed with one or more probes consisting essentially of SEQ ID NO: 43, SEQ ID NO:44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and/or SEQ ID NO: 55. In other embodiments, the real-time PCR can be performed with one or more probes consisting of SEQ ID NO: 43, SEQ ID NO:44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and/or SEQ ID NO: 55. In other embodiments, the real-time PCR can be performed with one or more probes having SEQ ID NO: 43, SEQ ID NO:44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, and/or SEQ ID NO: 55.

The qRT-PCR can be performed with one or more 3′ blocking oligonucleotides. Exemplary qRT-PCR procedures using 3′ blocking oligonucleotides are disclosed in the Example section herein. Suitable 3′ blocking oligonucleotides include, for example, those disclosed in Table 8. In some embodiments, the qRT-PCR can be performed with one or more 3′ blocking oligonucleotides comprising SEQ ID NO: 39, SEQ ID NO: 40, SEQ ID NO: 41, and/or SEQ ID NO: 42. In some embodiments, the qRT-PCR can be performed with one or more 3′ blocking oligonucleotides consisting essentially of SEQ ID NO: 39, SEQ ID NO: 40, SEQ ID NO: 41, and/or SEQ ID NO: 42. In some embodiments, the qRT-PCR can be performed with one or more 3′ blocking oligonucleotides consisting of SEQ ID NO: 39, SEQ ID NO: 40, SEQ ID NO: 41, and/or SEQ ID NO: 42. In some embodiments, the qRT-PCR can be performed with one or more 3′ blocking oligonucleotides having SEQ ID NO: 39, SEQ ID NO: 40, SEQ ID NO: 41, and/or SEQ ID NO: 42.

Suitable pairs of primers for use in the amplifying step include those disclosed in Table 3. For example, in some embodiments, the FGFR mutant and pair of primers can be FGFR3:TACC3 v1 and primers having the amino acid sequences of SEQ ID NO:5 and SEQ ID NO:6. In some embodiments, the FGFR mutant and pair of primers can be FGFR3:TACC3 v3 and primers having the amino acid sequences of SEQ ID NO:7 and SEQ ID NO:8. In some embodiments, the FGFR mutant and pair of primers can be FGFR3:TACC3 Intron and primers having the amino acid sequences of SEQ ID NO:9 and SEQ ID NO:10. In some embodiments, the FGFR mutant and pair of primers can be FGFR3: BAIAP2L1 and primers having the amino acid sequences of SEQ ID NO:11 and SEQ ID NO:12. In some embodiments, the FGFR mutant and pair of primers can be FGFR2:BICC1 and primers having the amino acid sequences of SEQ ID NO:13 and SEQ ID NO:14. In some embodiments, the FGFR mutant and pair of primers can be FGFR2:AFF3 and primers having the amino acid sequences of SEQ ID NO:15 and SEQ ID NO:16. In some embodiments, the FGFR mutant and pair of primers can be FGFR2:CASP7 and primers having the amino acid sequences of SEQ ID NO:17 and SEQ ID NO:18. In some embodiments, the FGFR mutant and pair of primers can be FGFR2:CCDC6 and primers having the amino acid sequences of SEQ ID NO:19 and SEQ ID NO:20. In some embodiments, the FGFR mutant and pair of primers can be FGFR2:OFD1 and primers having the amino acid sequences of SEQ ID NO:21 and SEQ ID NO:22. In some embodiments, the FGFR mutant and pair of primers can be R248C and primers having the amino acid sequences of SEQ ID NO:23 and SEQ ID NO:24 or SEQ ID NO:31 and SEQ ID NO:32. In some embodiments, the FGFR mutant and pair of primers can be S249C and primers having the amino acid sequences of SEQ ID NO:25 and SEQ ID NO:26 or SEQ ID NO:33 and SEQ ID NO:34. In some embodiments, the FGFR mutant and pair of primers can be G370C and primers having the amino acid sequences of SEQ ID NO:27 and SEQ ID NO:28 or SEQ ID NO:35 and SEQ ID NO:36. In some embodiments, the FGFR mutant and pair of primers can be Y373C and primers having the amino acid sequences of SEQ ID NO:29 and SEQ ID NO:30 or SEQ ID NO:37 and SEQ ID NO:38. In some embodiments, the FGFR mutant and pair of primers can be any combination of the above disclosed FGFR mutants and corresponding pair of primers.

In some embodiments, the amplifying step can be performed with the following:
a. the pair of primers have the sequences SEQ ID NO:5 and SEQ ID NO:6 and the probe has the sequence of SEQ ID NO:43;
b. the pair of primers have the sequences SEQ ID NO:7 and SEQ ID NO:8 and the probe has the sequence of SEQ ID NO:44;
c. the pair of primers have the sequences SEQ ID NO:9 and SEQ ID NO:10 and the probe has the sequence of SEQ ID NO:46;
d. the pair of primers have the sequences SEQ ID NO:11 and SEQ ID NO:12 and the probe has the sequence of SEQ ID NO:47;
e. the pair of primers have the sequences SEQ ID NO:13 and SEQ ID NO:14 and the probe has the sequence of SEQ ID NO:45;
f. the pair of primers have the sequences SEQ ID NO:15 and SEQ ID NO:16 and the probe has the sequence of SEQ ID NO:48;
g. the pair of primers have the sequences SEQ ID NO:17 and SEQ ID NO:18 and the probe has the sequence of SEQ ID NO:49;
h. the pair of primers have the sequences SEQ ID NO:19 and SEQ ID NO:20 and the probe has the sequence of SEQ ID NO:50;
i. the pair of primers have the sequences SEQ ID NO:21 and SEQ ID NO:22 and the probe has the sequence of SEQ ID NO:51;
j. the pair of primers have the sequences SEQ ID NO:23 and SEQ ID NO:24 and the probe has the sequence of SEQ ID NO:52;
k. the pair of primers have the sequences SEQ ID NO:25 and SEQ ID NO:26 and the probe has the sequence of SEQ ID NO:53;
l. the pair of primers have the sequences SEQ ID NO:27 and SEQ ID NO:28 and the probe has the sequence of SEQ ID NO:54;
m. the pair of primers have the sequences SEQ ID NO:29 and SEQ ID NO:30 and the probe has the sequence of SEQ ID NO:55;
n. the pair of primers have the sequences SEQ ID NO:31 and SEQ ID NO:32, the probe has the sequence of SEQ ID NO:52, and the 3′ blocking oligonucleotide has the sequence of SEQ ID NO:39;
o. the pair of primers have the sequences SEQ ID NO:33 and SEQ ID NO:34, the probe has the sequence of SEQ ID NO:53, and the 3′ blocking oligonucleotide has the sequence of SEQ ID NO:40;
p. the pair of primers have the sequences SEQ ID NO:35 and SEQ ID NO:36, the probe has the sequence of SEQ ID NO:54, and the 3′ blocking oligonucleotide has the sequence of SEQ ID NO:41;
q. the pair of primers have the sequences SEQ ID NO:37 and SEQ ID NO:38, the probe has the sequence of SEQ ID NO:55, and the 3′ blocking oligonucleotide has the sequence of SEQ ID NO:42; or
r. any combination thereof.

The disclosed methods comprise treating a patient if one or more FGFR mutants are present in the sample. The presence of one or more FGFR mutants in the sample can be determined by, for example, sequencing the amplified cDNA.

Suitable FGFR inhibitors for use in the treatment methods include those previously described herein.

Also disclosed are FGFR inhibitors for use in the treatment of cancer in a patient wherein the patient is identified as being responsive to treatment with the FGFR inhibitor by evaluating a biological sample obtained from the patient for the presence of one or more FGFR mutants from a FGFR mutant gene panel, wherein the presence of the one or more FGFR mutants in the sample is detected.

Further disclosed are FGFR inhibitors for use in the treatment of cancer in a patient wherein the patient is identified as being responsive to treatment with the FGFR inhibitor by evaluating a biological sample obtained from the patient for the presence of one or more FGFR mutants from a FGFR mutant gene panel, wherein the one or more FGFR mutants are a FGFR fusion gene or a FGFR SNP, wherein the presence of the one or more FGFR mutants in the sample is detected, and wherein said evaluating comprises amplifying cDNA with a pair of primers that bind to and amplify one or more FGFR mutants from the FGFR mutant gene panel.

Further disclosed are FGFR inhibitors for use in the treatment of cancer in a patient wherein the patient is identified as being responsive to treatment with the FGFR inhibitor by evaluating a biological sample obtained from the patient for the presence of one or more FGFR mutants from a FGFR mutant gene panel, wherein the FGFR mutant is a FGFR fusion gene or a FGFR SNP, wherein the presence of one or more FGFR mutants in the sample is detected, and wherein said evaluating comprises amplifying a pre-amplified cDNA with a pair of primers that bind to and amplify one or more FGFR mutants from the FGFR mutant gene panel.

Methods of Identifying a Cancer Patient that Will be Responsive to Treatment with a Fibroblast Growth Factor Receptor (FGFR) Inhibitor

Disclosed herein are methods of identifying a cancer patient that will be responsive to treatment with a fibroblast growth factor receptor (FGFR) inhibitor comprising: evaluating a biological sample from the patient for a FGFR mutant from a FGFR mutant gene panel, wherein the FGFR mutant is a FGFR fusion gene or a FGFR single nucleotide polymorphism, and wherein said evaluating comprises amplifying cDNA with a pair of primers that bind to and amplify one or more FGFR mutants from the FGFR mutant gene panel and determining whether the one or more FGFR mutants from the gene panel are present in the sample, wherein the presence of the one or more FGFR mutants indicates that the patient will be responsive to treatment with the FGFR inhibitor.

Also provided are methods of identifying a cancer patient that is responsive to treatment with a fibroblast growth factor receptor (FGFR) inhibitor comprising: evaluating a biological sample from the patient for a FGFR mutant from a FGFR mutant gene panel, wherein the FGFR mutant is a FGFR fusion gene or a FGFR single nucleotide polymorphism, and wherein said evaluating comprises amplifying cDNA with a pair of primers that bind to and amplify one or more FGFR mutants from the FGFR mutant gene panel and determining whether the one or more FGFR mutants from the gene panel are present in the sample, wherein the presence of the one or more FGFR mutants indicates that the patient is responsive to treatment with the FGFR inhibitor.

Further provided are methods of identifying a cancer patient that is responsive to treatment with a fibroblast growth factor receptor (FGFR) inhibitor comprising evaluating a biological sample from the patient for the presence of one or more FGFR mutant from a FGFR mutant gene panel, wherein the FGFR mutant is a FGFR fusion gene or a FGFR single nucleotide polymorphism, wherein the presence of the one or more FGFR mutants indicates that the patient is responsive to treatment with the FGFR inhibitor.

In some embodiments, the evaluating can comprise amplifying cDNA with a pair of primers that bind to and amplify one or more FGFR mutants from the FGFR mutant gene panel. In some embodiments, the cDNA can be pre-amplified cDNA.

In some embodiments, the evaluating step comprises: isolating RNA from the biological sample and synthesizing cDNA from the isolated RNA. In some aspects, the evaluating step can be performed on preamplified cDNA. Thus, the evaluating step can further comprise pre-amplifying the cDNA prior to said amplifying step. Isolating RNA from a biological sample can be performed by a number of procedures known to one skilled in the art. In one embodiment, RNA can be isolated from the biological sample using an AllPrep DNA/RNA FFPE Kit from Qiagen (for example, product #80234). Synthesizing cDNA from isolated RNA can be performed by a number of procedures known to one skilled in the art. In one embodiment, cDNA can be synthesized from isolated RNA using a High Capacity cDNA Reverse Transcriptase Kit with RNase Inhibitor from ABI (for example, product #4374966). Pre-amplification of cDNA can be performed by a number of procedures known to one skilled in the art. Amplification procedures are well known in the art. In one embodiment, cDNA can be pre-amplified using a TaqMan® PreAmp Master Mix (Life Technologies/Applied Biosystems® product #4391128).

The disclosed methods can be used to identify patients with a number of different types of cancer that will be responsive to treatment with a fibroblast growth factor receptor (FGFR) inhibitor including, but not limited to, bladder cancer, metastatic bladder cancer, ovarian cancer, head and neck cancer, esophageal cancer, non-small-cell lung adenocarcinoma, non-small cell lung squamous cell carcinoma, prostate cancer, lung cancer, gastric cancer, urothelial carcinoma, small cell lung cancer, breast cancer, endometrial cancer, cholangiocarcinoma, hepatocellular carcinoma, glioblastoma, gliomas, colon carcinoma, sarcomas, solid tumors of squamous origin, and multiple myeloma.

The FGFR mutant panel that is used in the evaluating step is based, in part, on the patient's cancer type. For patients with bladder cancer, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof. Accordingly, in some embodiments, the evaluating step comprises determining whether FGFR3:TACC3 v1 is present in a biological sample from a patient having bladder cancer. In some embodiments, the evaluating step comprises determining whether FGFR3:TACC3 v3 is present in a biological sample from a patient having bladder cancer. In some embodiments, the evaluating step comprises determining whether FGFR3:BAIAP2L1 is present in a biological sample from a patient having bladder cancer. In some embodiments, the evaluating step comprises determining whether FGFR2:BICC1 is present in a biological sample from a patient having bladder cancer. In some embodiments, the evaluating step comprises determining whether FGFR2:AFF3 is present in a biological sample from a patient having bladder cancer. In some embodiments, the evaluating step comprises determining whether FGFR2:CASP7 is present in a biological sample from a patient having bladder cancer. In some embodiments, the evaluating step comprises determining whether FGFR3 R248C is present in a biological sample from a patient having bladder cancer. In some embodiments, the evaluating step comprises determining whether FGFR3 S249C is present in a biological sample from a patient having bladder cancer. In some embodiments, the evaluating step comprises determining whether FGFR3 G370C is present in a biological sample from a patient having bladder cancer. In some embodiments, the evaluating step comprises determining whether FGFR3 Y373C is present in a biological sample from a patient having bladder cancer. In some embodiments, the evaluating step comprises determining whether any combination of the above FGFR mutants are present in a biological sample from a patient having bladder cancer.

For patients with metastatic bladder cancer, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof. Accordingly, in some embodiments, the evaluating step comprises determining whether FGFR3:TACC3 v1 is present in a biological sample from a patient having metastatic bladder cancer. In some embodiments, the evaluating step comprises determining whether FGFR3:TACC3 v3 is present in a biological sample from a patient having metastatic bladder cancer. In some embodiments, the evaluating step comprises determining whether FGFR3:BAIAP2L1 is present in a biological sample from a patient having metastatic bladder cancer. In some embodiments, the evaluating step comprises determining whether FGFR2:BICC1 is present in a biological sample from a patient having metastatic bladder cancer. In some embodiments, the evaluating step comprises determining whether FGFR2:AFF3 is present in a biological sample from a patient having metastatic bladder cancer. In some embodiments, the evaluating step comprises determining whether FGFR2:CASP7 is present in a biological sample from a patient having metastatic bladder cancer. In some embodiments, the evaluating step comprises determining whether FGFR3 R248C is present in a biological sample from a patient having metastatic bladder cancer. In some embodiments, the evaluating step comprises determining whether FGFR3 S249C is present in a biological sample from a patient having metastatic bladder cancer. In some embodiments, the evaluating step comprises determining whether FGFR3 G370C is present in a biological sample from a patient having metastatic bladder cancer. In some embodiments, the evaluating step comprises determining whether FGFR3 Y373C is present in a biological sample from a patient having metastatic bladder cancer. In some embodiments, the evaluating step comprises determining whether any combination of the above FGFR mutants are present in a biological sample from a patient having metastatic bladder cancer.

For patients with ovarian cancer, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, or FGFR3 Y373C, or any combination thereof. Accordingly, in some embodiments, the evaluating step comprises determining whether FGFR3:TACC3 v1 is present in a biological sample from a patient having ovarian cancer. In some embodiments, the evaluating step comprises determining whether FGFR3:TACC3 v3 is present in a biological sample from a patient having ovarian cancer. In some embodiments, the evaluating step comprises determining whether FGFR3:BAIAP2L1 is present in a biological sample from a patient having ovarian cancer. In some embodiments, the evaluating step comprises determining whether FGFR2:BICC1 is present in a biological sample from a patient having ovarian cancer. In some embodiments, the evaluating step comprises determining whether FGFR2:AFF3 is present in a biological sample from a patient having ovarian cancer. In some embodiments, the evaluating step comprises determining whether FGFR2:CASP7 is present in a biological sample from a patient having ovarian cancer. In some embodiments, the evaluating step comprises determining whether FGFR3 R248C is present in a biological sample from a patient having ovarian cancer. In some embodiments, the evaluating step comprises determining whether FGFR3 S249C is present in a biological sample from a patient having ovarian cancer. In some embodiments, the evaluating step comprises determining whether FGFR3 G370C is present in a biological sample from a patient having ovarian cancer. In some embodiments, the evaluating step comprises determining whether FGFR3 Y373C is present in a biological sample from a patient having ovarian cancer. In some embodiments, the evaluating step comprises determining whether any combination of the above FGFR mutants is present in a biological sample from a patient having ovarian cancer.

For patients with head and neck cancer, for example, a suitable FGFR mutant gene panel can comprise FGFR3:BAIAP2L1, FGFR2:CASP7, FGFR3 R248C, FGFR3 S249C, FGFR3 G370C, or FGFR3 Y373C, or any combination thereof. Accordingly, in some embodiments, the evaluating step comprises determining whether FGFR3:BAIAP2L1 is present in a biological sample from a patient having head and neck cancer. In some embodiments, the evaluating step comprises determining whether FGFR2:CASP7 is present in a biological sample from a patient having head and neck cancer. In some embodiments, the evaluating step comprises determining whether FGFR3 R248C is present in a biological sample from a patient having head and neck cancer. In some embodiments, the evaluating step comprises determining whether FGFR3 S249C is present in a biological sample from a patient having head and neck cancer. In some embodiments, the evaluating step comprises determining whether FGFR3 G370C is present in a biological sample from a patient having head and neck cancer. In some embodiments, the evaluating step comprises determining whether FGFR3 Y373C is present in a biological sample from a patient having head and neck cancer. In some embodiments, the evaluating step comprises determining whether any combination of the above FGFR mutants is present in a biological sample from a patient having head and neck cancer.

For patients with metastatic head and neck cancer, for example, a suitable FGFR mutant gene panel can comprise FGFR3:BAIAP2L1, FGFR2:CASP7, or FGFR2:OFD1, or any combination thereof. Accordingly, in some embodiments, a patient having metastatic head and neck cancer is treated with an FGFR inhibitor if FGFR3:BAIAP2L1 is present in the sample. In some embodiments, a patient having metastatic head and neck cancer is treated with an FGFR inhibitor if FGFR2:CASP7 is present in the sample. In some embodiments, a patient having metastatic head and neck cancer is treated with an FGFR inhibitor if FGFR2:OFD1 is present in the sample. In some embodiments, a patient having metastic head and neck cancer is treated with an FGFR inhibitor if any combination of the above FGFR mutants is present in the sample.

For patients with esophageal cancer, for example, a suitable FGFR mutant gene panel can comprise FGFR3:TACC3 v1, FGFR3:TACC3 v3, FGFR2:BICC1, FGFR2:CASP7, FGFR3 R248C, FGFR3 S249C, FGFR3 G370C, or FGFR3 Y373C, or any combination thereof. Accordingly, in some embodiments, the evaluating step comprises determining whether FGFR3:TACC3 v1 is present in a biological sample from a patient having esophageal cancer. In some embodiments, the evaluating step comprises determining whether FGFR3:TACC3 v3 is present in a biological sample from a patient having esophageal cancer. In some embodiments, the evaluating step comprises determining whether FGFR2:BICC1 is present in a biological sample from a patient having esophageal cancer. In some embodiments, the evaluating step comprises determining whether FGFR2:CASP7 is present in a biological sample from a patient having esophageal cancer. In some embodiments, the evaluating step comprises determining whether FGFR3 R248C is present in a biological sample from a patient having esophageal cancer. In some embodiments, the evaluating step comprises determining whether FGFR3 S249C is present in a biological sample from a patient having esophageal cancer. In some embodiments, the evaluating step comprises determining whether FGFR3 G370C is present in a biological sample from a patient having esophageal cancer. In some embodiments, the evaluating step comprises determining whether FGFR3 Y373C is present in a biological sample from a patient having esophageal cancer. In some embodiments, the evaluating step comprises determining whether any combination of the above FGFR mutants is present in a biological sample from a patient having esophageal cancer.

For patients with metastatic esophageal cancer, for example, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCD6, or FGFR2:OFD1, or any combination thereof. Accordingly, in some embodiments, a patient having metastatic esophageal cancer is treated with an FGFR inhibitor if FGFR3:TACC3 v1 is present in the sample. In some embodiments, a patient having metastatic esophageal cancer is treated with an FGFR inhibitor if FGFR3:TACC3 v3 is present in the sample. In some embodiments, a patient having metastatic esophageal cancer is treated with an FGFR inhibitor if FGFR3:TACC3 Intron is present in the sample. In some embodiments, a patient having metastatic esophageal cancer is treated with an FGFR inhibitor if FGFR3:BAIAP2L1 is present in the sample. In some embodiments, a patient having metastatic esophageal cancer is treated with an FGFR inhibitor if FGFR2:BICC1 is present in the sample. In some embodiments, a patient having metastatic esophageal cancer is treated with an FGFR inhibitor if FGFR2:AFF3 is present in the sample. In some embodiments, a patient having metastatic esophageal cancer is treated with an FGFR inhibitor if FGFR2:CASP7 is present in the sample. In some embodiments, a patient having metastatic esophageal cancer is treated with an FGFR inhibitor if FGFR2:CCD6 is present in the sample. In some embodiments, a patient having metastatic esophageal cancer is treated with an FGFR inhibitor if FGFR2:OFD1 is present in the sample. In some embodiments, a patient having metastatic esophageal cancer is treated with an FGFR inhibitor if any combination of the above FGFR mutants is present in the sample.

For patients with non-small-cell lung (NSCL) adenocarcinoma, for example, a suitable FGFR mutant gene panel can comprise FGFR3:TACC3 v1, FGFR3:TACC3 v3, FGFR3:TACC3 Intron, FGFR3:BAIAP2L1, FGFR2:AFF3, FGFR2:CASP7, FGFR3 R248C, FGFR3 S249C, FGFR3 G370C, or FGFR3 Y373C, or any combination thereof. Accordingly, in some embodiments, the evaluating step comprises determining whether FGFR3:TACC3 v1 is present in a biological sample from a patient having NSCL adenocarcinoma. In some embodiments, the evaluating step comprises determining whether FGFR3:TACC3 v3 is present in a biological sample from a patient having NSCL adenocarcinoma. In some embodiments, the evaluating step comprises determining whether FGFR3:TACC3 Intron is present in a biological sample from a patient having NSCL adenocarcinoma. In some embodiments, the evaluating step comprises determining whether FGFR3:BAIAP2L1 is present in a biological sample from a patient having NSCL adenocarcinoma. In some embodiments, the evaluating step comprises determining whether FGFR2:AFF3 is present in a biological sample from a patient having NSCL adenocarcinoma. In some embodiments, the evaluating step comprises determining whether FGFR2:CASP7 is present in a biological sample from a patient having NSCL adenocarcinoma. In some embodiments, the evaluating step comprises determining whether FGFR3 R248C is present in a biological sample from a patient having NSCL adenocarcinoma. In some embodiments, the evaluating step comprises determining whether FGFR3 S249C is present in a biological sample from a patient having NSCL adenocarcinoma. In some embodiments, the evaluating step comprises determining whether FGFR3 G370C is present in a biological sample from a patient having NSCL adenocarcinoma. In some embodiments, the evaluating step comprises determining whether FGFR3 Y373C is present in a biological sample from a patient having NSCL adenocarcinoma. In some embodiments, the evaluating step comprises determining whether any combination of the above FGFR mutants is present in a biological sample from a patient having NSCL adenocarcinoma.

For patients with non-small cell lung (NSCL) squamous cell carcinoma, for example, a suitable FGFR mutant gene panel can comprise FGFR3:TACC3 v1, FGFR3:TACC3 v3, FGFR3:BAIAP2L1, FGFR2:BICC1, FGFR2:AFF3, FGFR2:CASP7, FGFR2:CCDC6, FGFR3 R248C, FGFR3 S249C, FGFR3 G370C, or FGFR3 Y373C, or any combination thereof. Accordingly, in some embodiments, the evaluating step comprises determining whether FGFR3:TACC3 v1 is present in a biological sample from a patient having NSCL squamous cell carcinoma. In some embodiments, the evaluating step comprises determining whether FGFR3:TACC3 v3 is present in a biological sample from a patient having NSCL squamous cell carcinoma. In some embodiments, the evaluating step comprises determining whether FGFR3:BAIAP2L1 is present in a biological sample from a patient having NSCL squamous cell carcinoma. In some embodiments, the evaluating step comprises determining whether FGFR2:BICC1 is present in a biological sample from a patient having NSCL squamous cell carcinoma. In some embodiments, the evaluating step comprises determining whether FGFR2:AFF3 is present in a biological sample from a patient having NSCL squamous cell carcinoma. In some embodiments, the evaluating step comprises determining whether FGFR2:CASP7 is present in a biological sample from a patient having NSCL squamous cell carcinoma. In some embodiments, the evaluating step comprises determining whether FGFR2:CCDC6 is present in a biological sample from a patient having NSCL squamous cell carcinoma. In some embodiments, the evaluating step comprises determining whether FGFR3 R248C is present in a biological sample from a patient having NSCL squamous cell carcinoma. In some embodiments, the evaluating step comprises determining whether FGFR3 S249C is present in a biological sample from a patient having NSCL squamous cell carcinoma. In some embodiments, the evaluating step comprises determining whether FGFR3 G370C is present in a biological sample from a patient having NSCL squamous cell carcinoma. In some embodiments, the evaluating step comprises determining whether FGFR3 Y373C is present in a biological sample from a patient having NSCL squamous cell carcinoma. In some embodiments, the evaluating step comprises determining whether any combination of the above FGFR mutants is present in a biological sample from a patient having NSCL squamous cell carcinoma.

For patients with metastatic endometrial cancer, for example, a suitable FGFR mutant gene panel can comprise FGFR3:TACC3 v1, FGFR3:TACC3 v3, FGFR3:TACC3 Intron, FGFR3:BAIAP2L1, FGFR2:CASP7, FGFR2:CCDC6, or FGFR2:OFD1, or any combination thereof. Accordingly, in some embodiments, a patient having metastatic endometrial cancer is treated with an FGFR inhibitor if FGFR3:TACC3 v1 is present in the sample. In some embodiments, a patient having metastatic endometrial cancer is treated with an FGFR inhibitor if FGFR3:TACC3 v3 is present in the sample. In some embodiments, a patient having metastatic endometrial cancer is treated with an FGFR inhibitor if FGFR3:TACC3 Intron is present in the sample. In some embodiments, a patient having metastatic endometrial cancer is treated with an FGFR inhibitor if FGFR3:BAIAP2L1 is present in the sample. In some embodiments, a patient having metastatic endometrial cancer is treated with an FGFR inhibitor if FGFR2:CASP7 is present in the sample. In some embodiments, a patient having metastatic endometrial cancer is treated with an FGFR inhibitor if FGFR2:CCDC6 is present in the sample. In some embodiments, a patient having metastatic endometrial cancer is treated with an FGFR inhibitor if FGFR2:OFD1 is present in the sample. In some embodiments, a patient having metastatic endometrial cancer is treated with an FGFR inhibitor if any combination of the above FGFR mutants is present in the sample.

For patients with breast cancer, for example, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCD6, or FGFR2:OFD1, or any combination thereof. Accordingly, in some embodiments, a patient having breast cancer is treated with an FGFR inhibitor if FGFR3:TACC3 v1 is present in the sample. In some embodiments, a patient having breast cancer is treated with an FGFR inhibitor if FGFR3:TACC3 v3 is present in the sample. In some embodiments, a patient having breast cancer is treated with an FGFR inhibitor if FGFR3:TACC3 Intron is present in the sample. In some embodiments, a patient having breast cancer is treated with an FGFR inhibitor if FGFR3:BAIAP2L1 is present in the sample. In some embodiments, a patient having breast cancer is treated with an FGFR inhibitor if FGFR2:BICC1 is present in the sample. In some embodiments, a patient having breast cancer is treated with an FGFR inhibitor if FGFR2:AFF3 is present in the sample. In some embodiments, a patient having breast cancer is treated with an FGFR inhibitor if FGFR2:CASP7 is present in the sample. In some embodiments, a patient having breast cancer is treated with an FGFR inhibitor if FGFR2:CCD6 is present in the sample. In some embodiments, a patient having breast cancer is treated with an FGFR inhibitor if FGFR2:OFD1 is present in the sample. In some embodiments, a patient having breast cancer is treated with an FGFR inhibitor if any combination of the above FGFR mutants is present in the sample.

For patients with hepatocellular carcinoma, for example, a suitable FGFR mutant gene panel can comprise FGFR3:TACC3 v1, FGFR3:TACC3 v3, FGFR3:TACC3 Intron, FGFR3:BAIAP2L1, FGFR2:BICC1, FGFR2:AFF3, FGFR2:CASP7, FGFR2:CCDC6, FGFR2:OFD1, FGFR3 R248C, FGFR3 S249C, FGFR3 G370C, or FGFR3 Y373C, or any combination thereof. Accordingly, in some embodiments, a patient having hepatocellular carcinoma is treated with an FGFR inhibitor if FGFR3:TACC3 v1 is present in the sample. In some embodiments, a patient having hepatocellular carcinoma is treated with an FGFR inhibitor if FGFR3:TACC3 v3 is present in the sample. In some embodiments, a patient having hepatocellular carcinoma is treated with an FGFR inhibitor if FGFR3:TACC3 Intron is present in the sample. In some embodiments, a patient having hepatocellular carcinoma is treated with an FGFR inhibitor if FGFR3:BAIAP2L1 is present in the sample. In some embodiments, a patient having hepatocellular carcinoma is treated with an FGFR inhibitor if FGFR2:BICC1 is present in the sample. In some embodiments, a patient having hepatocellular carcinoma is treated with an FGFR inhibitor if FGFR2:AFF3 is present in the sample. In some embodiments, a patient having hepatocellular carcinoma is treated with an FGFR inhibitor if FGFR2:CASP7 is present in the sample. In some embodiments, a patient having hepatocellular carcinoma is treated with an FGFR inhibitor if FGFR2:CCDC6 is present in the sample. In some embodiments, a patient having hepatocellular carcinoma is treated with an FGFR inhibitor if FGFR2:OFD1 is present in the sample. In some embodiments, a patient having hepatocellular carcinoma is treated with an FGFR inhibitor if FGFR3 R248C is present in the sample. In some embodiments, a patient having hepatocellular carcinoma is treated with an FGFR inhibitor if FGFR3 S249C is present in the sample. In some embodiments, a patient having hepatocellular carcinoma is treated with an FGFR inhibitor if FGFR3 G370C is present in the sample. In some embodiments, a patient having hepatocellular carcinoma is treated with an FGFR inhibitor if FGFR3 Y373C is present in the sample. In some embodiments, a patient having hepatocellular carcinoma is treated with an FGFR inhibitor if any combination of the above FGFR mutants is present in the sample.

Suitable pairs of primers for use in the amplifying step include those disclosed in Table 3. For example, in some embodiments, the FGFR mutant and pair of primers can be FGFR3:TACC3 v1 and primers having the amino acid sequences of SEQ ID NO:5 and SEQ ID NO:6. In some embodiments, the FGFR mutant and pair of primers can be FGFR3:TACC3 v3 and primers having the amino acid sequences of SEQ ID NO:7 and SEQ ID NO:8. In some embodiments, the FGFR mutant and pair of primers can be FGFR3:TACC3 Intron and primers having the amino acid sequences of SEQ ID NO:9 and SEQ ID NO:10. In some embodiments, the FGFR mutant and pair of primers can be FGFR3:BAIAP2L1 and primers having the amino acid sequences of SEQ ID NO:11 and SEQ ID NO:12. In some embodiments, the FGFR mutant and pair of primers can be FGFR2:BICC1 and primers having the amino acid sequences of SEQ ID NO:13 and SEQ ID NO:14. In some embodiments, the FGFR mutant and pair of primers can be FGFR2:AFF3 and primers having the amino acid sequences of SEQ ID NO:15 and SEQ ID NO:16. In some embodiments, the FGFR mutant and pair of primers can be FGFR2:CASP7 and primers having the amino acid sequences of SEQ ID NO:17 and SEQ ID NO:18. In some embodiments, the FGFR mutant and pair of primers can be FGFR2:CCDC6 and primers having the amino acid sequences of SEQ ID NO:19 and SEQ ID NO:20. In some embodiments, the FGFR mutant and pair of primers can be FGFR2:OFD1 and primers having the amino acid sequences of SEQ ID NO:21 and SEQ ID NO:22. In some embodiments, the FGFR mutant and pair of primers can be R248C and primers having the amino acid sequences of SEQ ID NO:23 and SEQ ID NO:24 or SEQ ID NO:31 and SEQ ID NO:32. In some embodiments, the FGFR mutant and pair of primers can be S249C and primers having the amino acid sequences of SEQ ID NO:25 and SEQ ID NO:26 or SEQ ID NO:33 and SEQ ID NO:34. In some embodiments, the FGFR mutant and pair of primers can be G370C and primers having the amino acid sequences of SEQ ID NO:27 and SEQ ID NO:28 or SEQ ID NO:35 and SEQ ID NO:36. In some embodiments, the FGFR mutant and pair of primers can be Y373C and primers having the amino acid sequences of SEQ ID NO:29 and SEQ ID NO:30 or SEQ ID NO:37 and SEQ ID NO:38. In some embodiments, the FGFR mutant and pair of primers can be any combination of the above disclosed FGFR mutants and corresponding pair of primers.

The disclosed methods comprise determining whether the one or more FGFR mutants from the gene panel are present in the sample. In some embodiments, the determining step comprises sequencing the amplified cDNA.

In some embodiments, the method further comprises treating the patient with an FGFR inhibitor if the one or more FGFR mutants from the gene panel are present in the sample. Suitable FGFR inhibitors for use in the treatment methods include those previously described herein, in particular JNJ-42756493.

### Kits for Identifying the Presence of FGFR Mutant Genes

Further disclosed are kits for identifying the presence of one or more FGFR mutant genes in a biological sample comprising: pairs of primers having the sequences of SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or any combination thereof; and instructions for performing an assay to detect one or more FGFR mutant genes.

The kits can further comprise one or more probes, one or more 3′ blocking oligonucleotides, or both. In some embodiments, the kits can further comprise one or more probes, for example, any one or more of the probes disclosed in Table 15. In some embodiments, the kits can further comprise one or more 3′ blocking oligonucleotides, for example, any one or more of the 3′ blocking oligonucleotides disclosed in Table 8. In some embodiments, the kits can further comprise one or more probes and one or more 3′ blocking oligonucleotides. For example, in some embodiments, the kits can further comprise:
a. the pair of primers have the sequences SEQ ID NO:5 and SEQ ID NO:6 and the probe has the sequence of SEQ ID NO:43;
b. the pair of primers have the sequences SEQ ID NO:7 and SEQ ID NO:8 and the probe has the sequence of SEQ ID NO:44;
c. the pair of primers have the sequences SEQ ID NO:9 and SEQ ID NO:10 and the probe has the sequence of SEQ ID NO:46;
d. the pair of primers have the sequences SEQ ID NO:11 and SEQ ID NO:12 and the probe has the sequence of SEQ ID NO:47;
e. the pair of primers have the sequences SEQ ID NO:13 and SEQ ID NO:14 and the probe has the sequence of SEQ ID NO:45;
f. the pair of primers have the sequences SEQ ID NO:15 and SEQ ID NO:16 and the probe has the sequence of SEQ ID NO:48;
g. the pair of primers have the sequences SEQ ID NO:17 and SEQ ID NO:18 and the probe has the sequence of SEQ ID NO:49;
h. the pair of primers have the sequences SEQ ID NO:19 and SEQ ID NO:20 and the probe has the sequence of SEQ ID NO:50;
i. the pair of primers have the sequences SEQ ID NO:21 and SEQ ID NO:22 and the probe has the sequence of SEQ ID NO:51;
j. the pair of primers have the sequences SEQ ID NO:23 and SEQ ID NO:24 and the probe has the sequence of SEQ ID NO:52;
k. the pair of primers have the sequences SEQ ID NO:25 and SEQ ID NO:26 and the probe has the sequence of SEQ ID NO:53;
l. the pair of primers have the sequences SEQ ID NO:27 and SEQ ID NO:28 and the probe has the sequence of SEQ ID NO:54;
m. the pair of primers have the sequences SEQ ID NO:29 and SEQ ID NO:30 and the probe has the sequence of SEQ ID NO:55;
n. the pair of primers have the sequences SEQ ID NO:31 and SEQ ID NO:32, the probe has the sequence of SEQ ID NO:52, and the 3′ blocking oligonucleotide has the sequence of SEQ ID NO:39;
o. the pair of primers have the sequences SEQ ID NO:33 and SEQ ID NO:34, the probe has the sequence of SEQ ID NO:53, and the 3′ blocking oligonucleotide has the sequence of SEQ ID NO:40;
p. the pair of primers have the sequences SEQ ID NO:35 and SEQ ID NO:36, the probe has the sequence of SEQ ID NO:54, and the 3′ blocking oligonucleotide has the sequence of SEQ ID NO:41;
q. the pair of primers have the sequences SEQ ID NO:37 and SEQ ID NO:38, the probe has the sequence of SEQ ID NO:55, and the 3′ blocking oligonucleotide has the sequence of SEQ ID NO:42; or
r. any combination thereof.

### Oligonucleotide Probes

Also disclosed are oligonucleotide probes having the sequence of any one of SEQ ID NOs:43-55. In some embodiments, the oligonucleotide probe can have the sequence of SEQ ID NO:43. In some embodiments, the oligonucleotide probe can have the sequence of SEQ ID NO:44. In some embodiments, the oligonucleotide probe can have the sequence of SEQ ID NO:45. In some embodiments, the oligonucleotide probe can have the sequence of SEQ ID NO:46. In some embodiments, the oligonucleotide probe can have the sequence of SEQ ID NO:47. In some embodiments, the oligonucleotide probe can have the sequence of SEQ ID NO:48. In some embodiments, the oligonucleotide probe can have the sequence of SEQ ID NO:49. In some embodiments, the oligonucleotide probe can have the sequence of SEQ ID NO:50. In some embodiments, the oligonucleotide probe can have the sequence of SEQ ID NO:51. In some embodiments, the oligonucleotide probe can have the sequence of SEQ ID NO:52. In some embodiments, the oligonucleotide probe can have the sequence of SEQ ID NO:53. In some embodiments, the oligonucleotide probe can have the sequence of SEQ ID NO:54. In some embodiments, the oligonucleotide probe can have the sequence of SEQ ID NO:55.

### 3′ Blocking Oligonucleotide

Also disclosed herein are oligonucleotides having the sequence of any one of SEQ ID NOs:39-42. In some embodiments, the 3′ blocking oligonucleotide can have the sequence of SEQ ID NO:39. In some embodiments, the 3′ blocking oligonucleotide can have the sequence of SEQ ID NO:40. In some embodiments, the 3′ blocking oligonucleotide can have the sequence of SEQ ID NO:41. In some embodiments, the 3′ blocking oligonucleotide can have the sequence of SEQ ID NO:42.

### EXAMPLES

### Example 1—Plasmid DNA Isolation and Purification

Below is an exemplary procedure for preparing FGFR fusion plasmid DNA.

Required equipment: centrifuge, capable of 1500× g; microcentrifuge; pipettors, positive-displacement or air-displacement; vortexer; nanodrop Spectrophotometer; 37° C. shaker/incubator; and an oven set to 37° C.

Required materials: frozen glycerol bacterial stock containing plasmid DNA; Kanamycin LB agar plates (Teknova # L1155); LB broth (Life Technologies #10855-021); Kanamycin (Sigma # K0254); plasmid purification kit (Qiagen #12123); absolute ethanol (Sigma Aldrich # E7023); isopropanol (Sigma Aldrich # W292907); Nuclease Free Water (Non-DEPC treated) (from IDT or Ambion # AM9932); RNase-free Barrier (Filter) Tips; RNase-free Microtube (1.5 to 2 mL VWR #10011-724); serological pipettes; and 14 ml Round bottom tubes (VWR #352057).

To recover bacteria from the glycerol stock, frozen bacteria were scraped off of the top of a glycerol stock tube using a sterile pipet tip, streaked onto a LB agar plate, and placed upside down in the oven at 37° C. overnight.

DNA plasmids were purified using Qiagen Plasmid DNA Purification protocol. Briefly, a single colony was picked from the streaked plate and incubated in a culture of 5 ml-LB medium containing 50 μg/ml Kanamycin overnight in a 37° C. shaker at approximately 300 rpm. The bacterial cells were harvested by centrifugation at 6000×g for 15 minutes at 4° C., and the pellet was resuspended in 300 μl of buffer P1. 300 μl of buffer P2 was added, mixed by inverting the tube 4-6 times, and incubated at RT (room temperature) for 5 minutes. 300 μl of chilled buffer P3 was added, mixed immediately by inverting 4-6 times, incubated on ice for 5 minutes, and centrifuged at maximum speed for 10 minutes. Supernatant containing plasmid DNA was removed promptly. A Qiagen-tip 20 was equilibrated by applying 1 ml of buffer QBT and allowed to empty by gravity flow. The supernatant was applied to the Qiagen-tip 20 and allowed to enter the resin by gravity flow. The Qiagen-tip 20 was washed with 2×2 ml of buffer QC and the DNA was eluted with 800 μl of buffer QF and the eluate was collected in a 1.5 ml Eppendorf tube. The DNA was precipitated by adding 0.7 volumes of isopropanol, mixed, and centrifuged immediately at 15000×g for 30 minutes in a microcentrifuge. The supernatant was decanted, and the DNA pellet was washed in 1 ml of 70% ethanol and centrifuged at 15000×g for 10 minutes. The supernatant was decanted. The pellet was air-dried for 5-10 minutes and the DNA was re-dissolved in 100 μl or suitable volume of nuclease-free water. Plasmid DNA was quantitated by Nanodrop and stored at −20° C. until further use.

### Example 2—Generation of NRK Cell Lines

Expression vectors expressing each of the FGFR fusions were constructed. The expression vector was then transfected into normal rat kidney epithelial cells (NRK) cells. The stable cell lines were selected in media containing kanamycin following transfections. These cells were then grown and mRNA was isolated and subjected to FGFR fusion assays to confirm the presence of the specific FGFR fusions mRNA.

### Example 3—FGFR-Fusion Cell Line Maintenance

The below protocol describes an exemplary procedure for culturing and maintaining the NRK FGFR-fusion over-expressing cell lines. Cell lines include, but are not limited to: NRK/FGFR3:TACC3v1, NRK/FGFR3:TACC3 v3, NRK/FGFR3:BAIAP2L1, NRK/FGFR2: BICC1, NRK/FGFR2:CASP7, NRK/FGFR2:CCDC6, NRK/FGFR2:AFF3, NRK/FGFR2:OFD1, and NRK/EMPTY VECTOR (plasmid control).

Required equipment: biosafety cabinet, fitted with vacuum aspiration system; CO₂ Incubator, set to 37° C. with 5% CO₂; −80° C. freezer; liquid nitrogen tank; water bath, set to 37° C.; and a microscope.

Required materials: serological pipettes; tissue culture flasks (T75 VWR # BD353136 and/or T150 VWR #15705-074); tissue culture 0.2 μm filtering units (Thermo Scientific #566-0020); DMEM (Dulbecco's Modified Eagle Medium) cell culture media (Life Technologies, #11965-084); Fetal Bovine Serum (FBS),certified, heat inactivated (Life Technologies, #10082147); PenStrep antibiotic solution (Life Technologies #15140-122); Trypsin-EDTA 0.25% solution (Life Technologies, #25200-056); DPBS (Dulbecco's Phosphate buffered solution, no calcium, no magnesium) (Life Technologies, #14190136); cell freezing container for cryopreservation; hand held pipetman; cell freezing media (Life Technologies, #12648-010); 15 ml conical tubes (VWR #62406-2); and cryovials (VWR #89094-800).

To prepare the cell culture media, DMEM medium was prepared by combining 445 ml of DMEM, 50 ml of FBS, and 5 ml of PenStrep. The prepared media was passed through a 0.2 μm filter unit and stored at 4° C.

To thaw frozen cells, prepared DMEM medium was warmed in the 37° C. water bath for at least 15 minutes and 15 ml of warmed medium was placed into a T75 flask. Cells were removed from liquid nitrogen tank and placed immediately in a 37° C. water bath until just thawed. Cryovials were sprayed generously with 70% alcohol and the excess was wiped with paper towels. The entire content was aliquoted into the T75 flask containing DMEM. Flask was swirled gently to mix and placed in incubator for 24 hours. If the cells were not ready for splitting, the media was changed to freshly prepared DMEM to remove residual freezing media. If cells were ready to split, each cell line was propagated once the flask achieved 80% confluency (splitting ratio for each cell line was dependent upon the experimental needs).

To freeze the cell lines, the cells were removed from the culture flask and spun down in a 15 ml conical tube for 5 minutes at 1500 RPM at RT. The medium was aspirated and 6 ml of cell freezing medium was added. The cells were mixed by pipetting up and down several times, and 1 ml of cell solution was aliquoted into each of 5 cryovials. Cryovials with cells were placed in a cryofreezing container, which was stored in a −80° C. freezer overnight, followed by long term storage in a liquid nitrogen tank.

### Example 4—FFPET SNP Assay

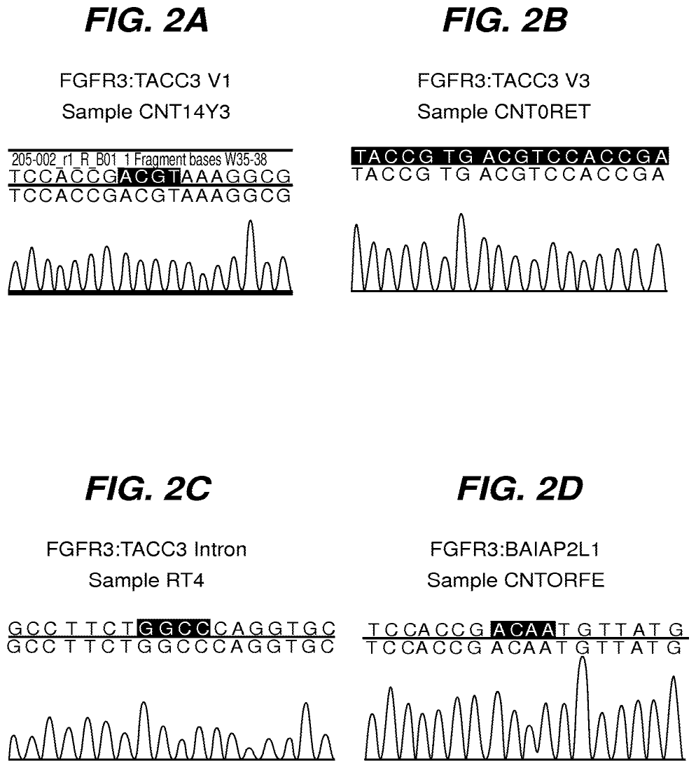

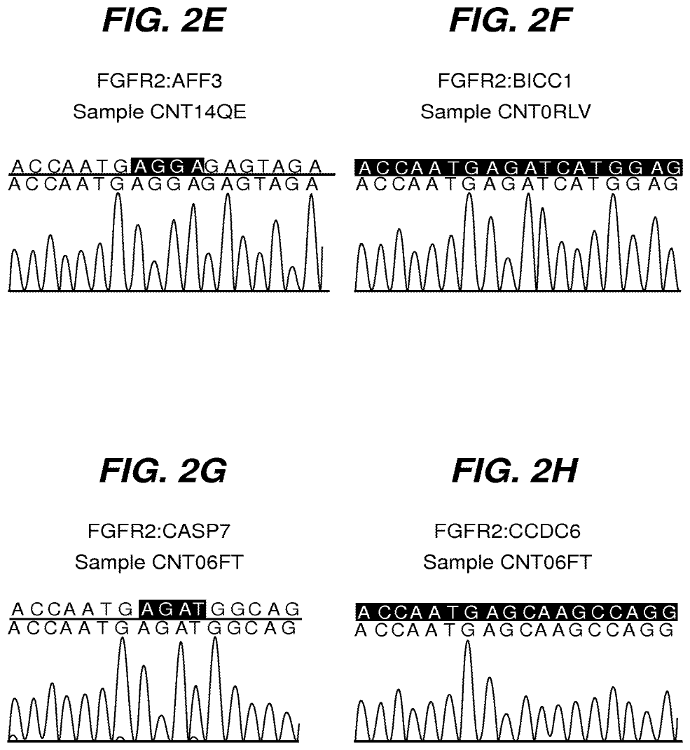

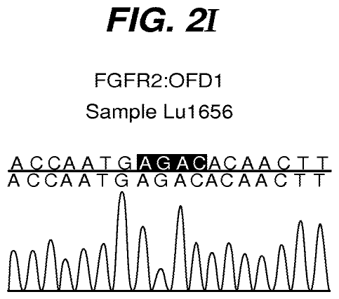

An exemplary workflow and protocol for performing a FFPET SNP assay is described below. A similar procedure is performed for FFPET fusion assays, the results of which are shown in FIGS. 2A-2I.

### De-Parafinization of FFPET

Slides were subjected to increasing amounts of xylene followed by alcohol treatment in order to remove the paraffin.

### FFPET RNA Extraction

The procedure for extracting RNA from breast cancer formalin fixed paraffin embedded tissue samples for downstream gene expression assay is described below.

Required equipment: centrifuge with plate adapter, capable of 1500×g; microcentrifuge; pipettors, positive-displacement or air-displacement; vortexer; NanoDrop 8000; heating block capable of incubation at 37° C., 56° C. and 80° C.; and pasteur pipette (Pipet Trans EX-FT 1.5 ml pk 500, VWR #14670-329).

Required Materials: AllPrep DNA/RNA FFPE Kit (Qiagen #80234); Absolute Ethanol (Sigma Aldrich # E7023); Isopropanol; Xylene; Nuclease Free Water (Non-DEPC treated) (from IDT or Ambion # AM9932); RNase-free Barrier (Filter) Tips; RNase-free; microtube (1.5 to 2 mL VWR #10011-724); and Qiagen AllPrep DNA/RNA FFPE Kit Handbook.

RNA was extracted using the AllPrep DNA/RNA FFPE Kit. Briefly, one 1-10 μm section was placed in a 1.5 ml reaction tube and 800 μl of HemoDe or Xylene were added. The sample was vortexed for 4 seconds 3 times, incubated for 2 minutes, vortexed for 4 seconds for 3 times and incubated for 5 minutes.

The sample was centrifuged for 2 minutes at maximum speed (12,000-14,000×g) and the supernatant was discarded by aspiration. Tubes were capped immediately to avoid tissue from drying.

The above steps were repeated.

800 μl ethanol abs. was added, the tube was flicked to dislodge the pellet, vortexed for 4 seconds 3 times, centrifuged for 2 minutes at maximum speed (12,000-14,000×g), and the supernatant was discarded by aspiration.

800 μl 70% ethanol was added, the tube was flicked to dislodge the pellet, vortexed for 4 seconds 3 times, centrifuged for 2 minutes at maximum speed, and the supernatant was discarded by aspiration. After removal of 70% ethanol, the tube was re-spun for 10-20 seconds and the residual fluid was carefully removed with a fine bore pipet.

The open tubes were incubated in a heating block for 5-15 minutes at 37° C. to air dry the tissue pellet.

The pellet was resuspended by adding 150 μl Buffer PKD and the tube was flicked to loosen the pellet. 10 μl proteinase K was added and the tube was mixed by vortexing.

Tubes were incubated at 56° C. for 15 minutes, incubated on ice for 3 minutes, and centrifuged for 15 minutes at 20,000×g.

The supernatant was carefully transferred without disturbing the pellet to a new 1.5 ml microcentrifuge tube for RNA purification. The supernatant was incubated at 80° C. for 15 minutes. The tube was briefly centrifuged to remove drops from the inside of the lid. 320 μl Buffer RLT was added to adjust binding conditions, and the tube was mixed by vortexing or pipetting. 1120 μl ethanol (96-100%) was added and the tube was mixed well by vortexing or pipetting.

700 μl of the sample, including any precipitate that may have formed, was transferred to an RNeasy MinElute spin column placed in a 2 ml collection tube, and centrifuged for 15 seconds at ≥8000×g (≥10,000 rpm). The flow-through was discarded. This step was repeated until the entire sample was passed through the RNeasy MinElute spin column.

350 μl Buffer FRN was added to the RNeasy MinElute spin column and centrifuged for 15 seconds at ≥8000×g (≥10,000 rpm). Flow-through was discarded.

10 μl DNase I stock solution was added to 70 μl RDD, mixed by gently inverting the tube, and centrifuged briefly to collect residual liquid from the sides of the tube.

The DNase I incubation mix (80 μl) was added directly to the RNeasy MinElute spin column membrane, and placed on the benchtop (20-30° C.) for 15 minutes.

500 μl Buffer FRN was added to the RNeasy MinElute spin column and centrifuged for 15 seconds at ≥8000×g (≥10,000 rpm). The flow-through was saved for use in the next step, as it contains small RNAs.

The RNeasy MinElute spin column was placed in a new 2 ml collection tube (supplied). The flow-through from the previous step was applied to the spin column and centrifuged for 15 seconds at ≥8000×g (≥10,000 rpm). Flow-through was discarded.

500 μl Buffer RPE was added to the RNeasy MinElute spin column and centrifuged for 15 second at ≥8000×g (≥10,000 rpm) to wash the spin column membrane. Flow-through was discarded.

500 μl Buffer RPE was added to the RNeasy MinElute spin column and centrifuged for 15 seconds at ≥8000×g (≥10,000 rpm) to wash the spin column membrane. Collection tube with the flow-through was discarded.

The RNeasy MinElute spin column was placed in a new 2 ml collection tube and centrifuged at full speed for 5 minutes. The collection tube with the flow-through was discarded.

The RNeasy MinElute spin column was placed in a new 1.5 ml collection tube, 30 μl RNase-free water was added directly to the spin column membrane, incubated for 1 minute at room temperature, and centrifuged at full speed for 1 minute to elute the RNA.

The RNA samples were immediately stored in −80° C. freezer.

cDNA Synthesis

Disclosed below is a procedure of cDNA synthesis for the FFPET SNP Assays using Real time PCR (RT-PCR) analysis.

Required equipment: centrifuge with plate adapter, capable of 1500×g, microcentrifuge; pipettors (preferred single and multi-channel pipettor), positive-displacement or air-displacement; vortexer; and GeneAmp® PCR System 9700 (ABI #4314879) or equivalent.

Required materials: High Capacity cDNA Reverse Transcriptase Kit with RNase Inhibitor, 200 reactions (ABI #4374966); Nuclease Free Water (Non-DEPC treated) (from IDT) or equivalent; RNase-free Barrier (Filter) Tips; RNase-free Microtube (1.5 to 2 mL VWR #10011-724); MicroAmp™ Optical 96-Well Reaction Plates (Life Technologies, #4306736); and sealing film (VWR #60941-072).

Following the RNA extraction (disclosed above) RNA sample tube(s) were kept on ice.

The kit components were used to prepare 2× Reverse Transcription (RT) Master Mix for all reactions, including 1 negative (water) control. Components were thawed on ice for approximately 15 minutes, gently inverted to mix and centrifuged briefly to bring down the solution. All reagents were returned to the ice. Tubes were not vortexed.

One Master Mix was prepared on ice in a 1.5 ml tube for the appropriate number of reactions (# reactions+10%, per 20-μL reaction) by combining the following amount of reagent per one reaction: 2 μl 10×RT Buffer Mix; 0.8 μl 25× dNTP Mix; 2 μl 10×RT Random Primers; 1 μl 50 U/μL MultiScribe Reverse Transcriptase; 1 μl RNase inhibitor; and 3.2 μl Nuclease/RNase free H₂0.

The Master Mix was vortexed several times (5 to 10) to mix and centrifuged briefly (1500×g, 5 to 10 seconds). 10 μl of the reaction mix was added to the appropriate wells of a 96-well plate.

The RNA samples were diluted to a concentration of 20 ng/μl. 10 μL of each RNA sample was added, including the water negative control, to the appropriate corresponding wells of the 96-well plate to a final reaction volume of 20 μL. The wells were mixed gently by pipetting up and down 3 times, sealed with a plate seal, and centrifuged briefly (1500×g for 60 seconds). Plates were kept on ice until ready to load in thermocycler.

The reaction plate was loaded into ABI 9700 Thermal Cycler in Clean Lab or Workstation and run using the following reverse-transcription program with a reaction volume of 20 μl:

Step 1: 25° C. for 10 minutes

Step 2: 37° C. for 120 minutes

Step 3: 85° C. for 5 seconds

Step 4: 4° C. infinite hold

Synthesized cDNA was stored at −20° C. for next step of Pre-amplification.

### Preamplification Assay Pool Mixture Preparation

The preamp assay pool mixture associated with the FFPET SNP Assay Pre-amplification Protocol was prepared as described below.

Required equipment: microcentrifuge; pipettors, positive-displacement or air-displacement; and vortexer.

Required materials: Nuclease Free Water (Non-DEPC treated) (from IDT) or equivalent; IDTE pH 8.0 (1× TE Solution) (IDT Technologies); RNase-free Barrier (Filter) Tips; and RNase-free Tubes (1.5 to 2 mL VWR #10011-724).

All TaqMan SNP Assays are ordered from Applied Biosystems, Life Technologies, Inc.

100 μL of 20×SNP assays were prepared.

To prepare 0.2× Preamp Assay Pool, all assays were thawed on ice for approximately 15 minutes. The following volume of components was added to a 1.5 ml tube:

TABLE 4

| | | |
|---|---|---|
| | Stock | Volume Needed for 200 ul |
| Target | Concentration | Preamp Stock (ul) |

Preamp Stock 1

| | | |
|---|---|---|
| FGFR3 S249C | 20X | 2 |
| IDTE | | 198 |
| Total Volume | | 200 |

Preamp Stock 2

| | | |
|---|---|---|
| FGFR3 R248C | 20X | 2 |
| IDTE | | 198 |
| Total Volume | | 200 |

Preamp Stock 3

| | | |
|---|---|---|
| FGFR3 Y373C | 20X | 2 |
| IDTE | | 198 |
| Total Volume | | 200 |

Note:
The above volumes are for the preparation of 200 μl of 0.2X preamp assay pool. Volumes can be adjusted accordingly depending on the number of samples being tested.

The 0.2× PreAmp Assay Pool was vortexed briefly to mix (5 to 10 seconds) and centrifuged briefly (1500×g, 5-10 seconds). 100 μL of PreAmp Primer Pool was aliquoted into 1.5 ml tubes and stored at −20° C.

### Pre-Amplification for the Breast Cancer Formalin-Fixed Paraffin Embedded Tissue SNP Assay Using Real Time PCR (RT-PCR) Analysis

Required equipment: centrifuge with plate adapter, capable of 1500×g; microcentrifuge; pipettors, positive-displacement or air-displacement; vortexer; GeneAmp® PCR System 9700 (ABI #4314879) or equivalent.

Required Materials: TaqMan® PreAmp Master Mix (2×) (Life Technologies #4391128); 0.2× Pooled Assay Mix (see Assay Preparation and Handling Protocol); 1×IDTE Buffer (10 mM Tris/0.1 mM EDTA, pH7.5, from IDT) or equivalent; Nuclease Free Water (Non-DEPC treated) (from IDT) or equivalent; RNase-free Barrier (Filter) Tips; RNase-free Microtube (1.5 to 2 mL VWR #10011-724); MicroAmp™ Optical 96-Well Reaction Plates (Life Technologies, #4306736); MicroAmp® Optical Adhesive Film (Applied Biosystems PN 4311971); deep well plates (VWR #47734-788); foil seals (VWR #60941-126).

Samples were prepared by placing the cDNA and 0.2× assay mix pool on ice to thaw, approximately 5 minutes, and centrifuging the plate briefly (1500×g for 5 to 10 seconds).

The kit components were used to prepare 2× PreAmp Master Mix. The kit components were allowed to thaw on ice for approximately 5 minutes. After all reagents were thawed, the tubes were gently inverted to mix and briefly centrifuged to bring down the solution. All reagents were returned to the ice. The tubes were not vortexed.

In a Clean Lab or Biosafety hood, each Master Mix was prepared for the appropriate number of reactions on ice by combining the required volumes of reagents as indicated Table 5 below (# reactions+10%):

TABLE 5

| | |
|---|---|
| Component | Volume (μL) for One Reaction |

Master Mix 1

| | |
|---|---|
| 2X TaqMan PreAmp Master Mix | 12.5 |
| 0.2X Assay Pool 1 | 6.25 |
| Total volume | 18.75 |

Master Mix 2

| | |
|---|---|
| 2X TaqMan PreAmp Master Mix | 12.5 |
| 0.2X Assay Pool 2 | 6.25 |
| Total volume | 18.75 |

Master Mix 3

| | |
|---|---|
| 2X TaqMan PreAmp Master Mix | 12.5 |
| 0.2X Assay Pool 3 | 6.25 |
| Total volume | 18.75 |

Assay pools contain primers and probes.

To prevent cross-priming of SNP assays, all 5 assays were split into 3 preamp reaction per sample.

Each Master Mix was vortexed several times (5 to 10) to mix, followed by a brief centrifuge (1500×g, 5 to 10 seconds). 18.75 μL of each Master Mix was aliquoted to the appropriate wells in a 96-well reaction plate. 6.25 μL of each cDNA samples, including water negative control well, was transferred into the appropriate wells in the Master Mix reaction plate for each preamp reaction. The sample was mixed gently by pipetting up and down 3 times and the cap was closed. The plate was briefly centrifuged (1500×g for 60 seconds) and kept on ice until ready to load in thermocycler.

The reaction plate ABI 9700 Thermal Cycler was loaded and run using the following program:

Step 1: 95° C. for 10 minutes

Step 2: 95° C. for 15 seconds

Step 3: 60° C. for 4 minutes

Step 4: Set Step 2-3 for 10 cycles
If a gold or silver block was used, max mode was selected and Ramp rate was set at 77%. If an aluminum block was used, standard mode (no rate change) was selected.

Step 5: 4° C. infinite hold

Reaction Volume Set to 25 μL

The PreAmp reaction plate was centrifuged briefly (1500×g for 60 seconds) after PreAmp completion. 100 μl of IDTE was added to the appropriate wells of a new deep 96-well plate and 25 μL of each PreAmp product was transferred to the corresponding wells to have final dilution volume of 125 μL. The each well was mixed by pipetting up and down 3 times, the plate was sealed with foil adhesive, the plate was centrifuged briefly (1500×g for 5 to 10 seconds), and the PreAmp product was stored at −20° C. until further use.

### FFPET SNP Assay—Real Time PCR

Disclosed below is the procedure for the Formalin-Fixed Paraffin Embedded Tissue SNP Assay using Real time PCR analysis.

Required equipment: centrifuge with plate adapter, capable of 1500×g; microcentrifuge; pipettors (preferred single and multi-channel pipettor), positive-displacement or air-displacement; vortexer; and ABI ViiA 7 real time PCR instrument (Life Technologies).

Required materials: TaqMan Genotyping Master Mix (Life Technologies #4371355); SNP Assays; Nuclease Free Water (Non-DEPC treated, from IDT) or equivalent; RNase-free Barrier (Filter) Tips; RNase-free Microtube (1.5 to 2 mL VWR #10011-724); MicroAmp® Optical Adhesive Film (Applied Biosystems PN 4311971); and MicroAmp™ Optical 384-Well Reaction Plates.

Table 15 lists the sequences of the probes used during the Real Time PCR assays.

To prepare the samples, in a Clean Lab or Workstation, SNP assays were placed on ice to thaw for approximately 5 minutes. All reagents protected from light, to protect exposure of the fluorescent probes. Diluted PreAmp plates were placed on ice to thaw in a Dirty Lab or Workstation after preparing Genotyping Master Mix.

To prepare genotyping master mix, the Genotyping Master Mix was thawed on ice for approximately 5 minutes. The Master Mix (MM) was prepared in the required number of tubes on ice. The required volumes of reagents were combined in the appropriate labeled tubes as indicated in Table 6 below (# reactions+10%):

TABLE 6

| Component | Volume (μL) for One Reaction |
|---|---|
| 2X Genotyping Master Mix | 10 |
| 20X SNP Assay | 1 |
| RNase-free Water | 4 |
| Total volume | 15 |

20X SNP assay mix contains primers, probes, and blocking oligos.

The Master Mix was vortexed several times (5 to 10) to mix and then centrifuged briefly (1500×g, 5 to 10 seconds). 15 μl of each Master Mix was added to the appropriate wells of a MicroAmp™ Optical 384-Well Reaction Plates. The reaction plates were sealed with optical adhesive film.

The plate with 1:5 diluted PreAmp product was placed on ice for approximately 5-10 minutes to thaw. Using a multi-channel pipettor, 5 μL of each diluted PreAmp product was transferred to the appropriate corresponding wells. The reaction plate was sealed with optical adhesive film and centrifuged briefly (1500×g for 60 seconds). Plates were kept on ice until ready to load in thermocycler.

The following conditions were run using the viiA 7 Software with the volume set at 20 μl:

TABLE 7

| Stage | Repetitions | Process | Temperature | Time | |
|---|---|---|---|---|---|
| 1 | 1 | Initial | 60° C. | 0.5 | minutes |
| 2 | 1 | DNApol Activation | 95° C. | 10 | minutes |
| 3 | 40 | Denature | 95° C. | 15 | seconds |
| | | Anneal/Extend | 60° C. | 1 | minutes |
| 4 | 1 | Post-Read | 60° C. | 30 | seconds |

FGFR SNP-Specific qRT-PCR

The detection of rare somatic mutations in an excess of wild type alleles is increasingly important in cancer diagnosis. When the mutations of interest are close to each other, detection becomes challenging. To aid in the identification of FGFR SNPs from FFPET, a SNP-specific qRT-PCR assay was developed, in which SNP-specific amplification using Taqman MGB probes combined with the 3′ dideoxy wild type (WT) allele blocker was used. The assay prevented non-specific binding, improved the number of on-target amplification, minimized the false positive signals from the WT alleles, and increased the sensitivity of the assay. This RNA based SNP detection assay, combined with the pre-amplification step in the assay, boosts the low or the rare mutant signals.

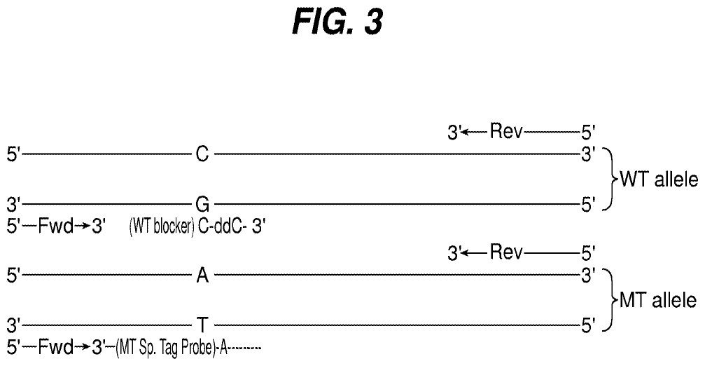

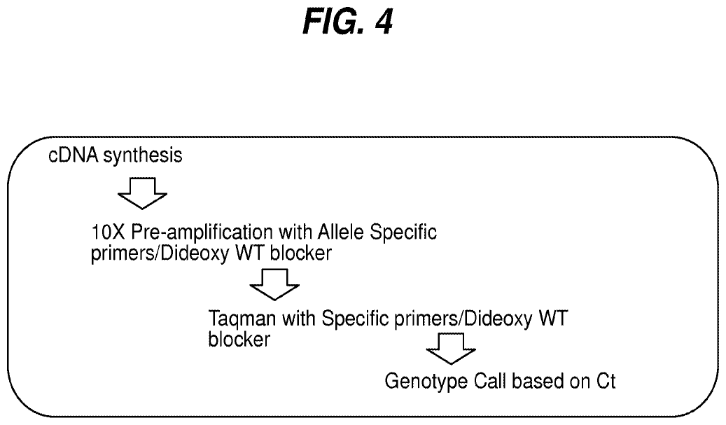

An exemplary strategy for SNP-specific qRT-PCR using a 3′ dideoxy WT blocker oligonucleotide is shown in FIG. 3, and an exemplary FFPE sample validation strategy is illustrated in FIG. 4. Briefly, qRT-PCR was performed using the FGFR SNP primers in the presence of a 3′ dideoxy WT blocker oligonucleotide, which was complementary to, and contained a short stretch of nucleotides flanking, the WT allele. Binding of the blocker oligonucleotide to the WT allele prevented applification of the WT allele, while the FGFR SNP primers bound to and specifically amplified the FGFR SNP. The 3′ dideoxy WT blocker oligonucleotides used in the FGFR SNP-specific qRT-PCR are shown in Table 8. The FGFR SNP primers used in the FGFR SNP-specific qRT-PCR were: SEQ ID NO:31 and SEQ ID NO:32 (FGFR3 R248C); SEQ ID NO:33 and SEQ ID NO:34 (FGFR3 S249C); SEQ ID NO:35 and SEQ ID NO:36 (FGFR3 G370C); and SEQ ID NO:37 and SEQ ID NO:38 (FGFR3 Y373). Table 15 lists the sequences of the probes used during the real time PCR assays.

TABLE 8

| | 3′ dideoxy WT blocker |
|---|---|
| Target | oligonucleotide |
| FGFR3 R248C | TGGAGCGCTCCCCGCA-ddC |
| | (SEQ ID NO: 39) |
| FGFR3 S249C | GACGTGCTGGAGYGCTC-ddC |
| | (SEQ ID NO: 40)* |
| FGFR3 G370C | CTGACGAGGCGGGCAG-ddC |
| | (SEQ ID NO: 41) |
| FGFR3 Y373 | GTGTGTATGCAGGCATCCTCAG-ddC |
| | (SEQ ID NO: 42) |

*Y can be T or C. 3′ WT blocking oligo will have 50% T and 50% C at that particular position during the synthesis (purified by manufacturer to provide T or C at that particular position).

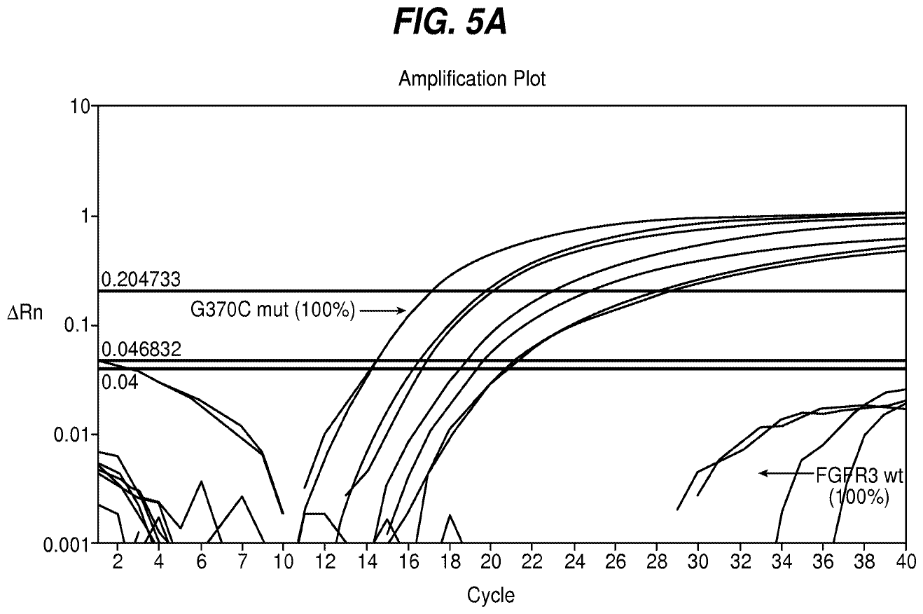

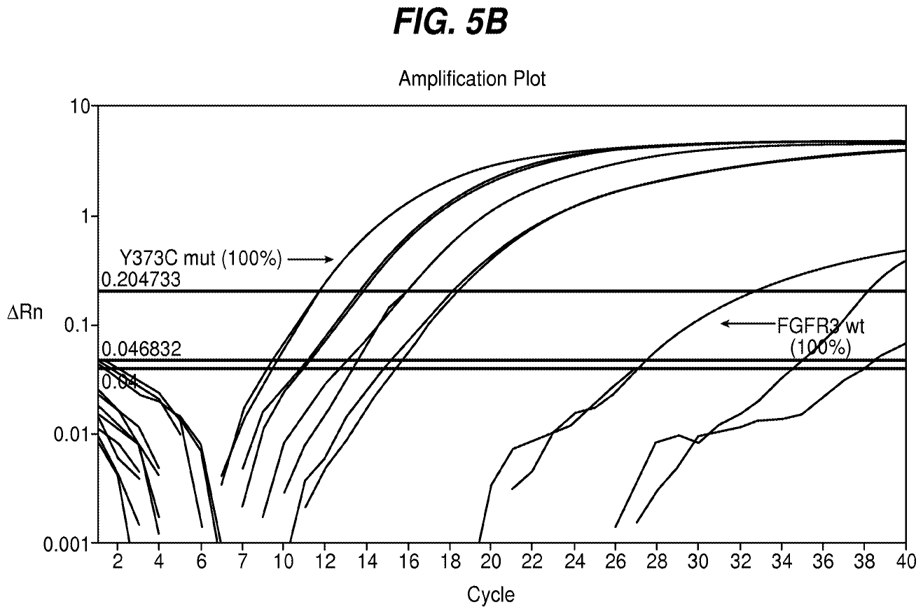

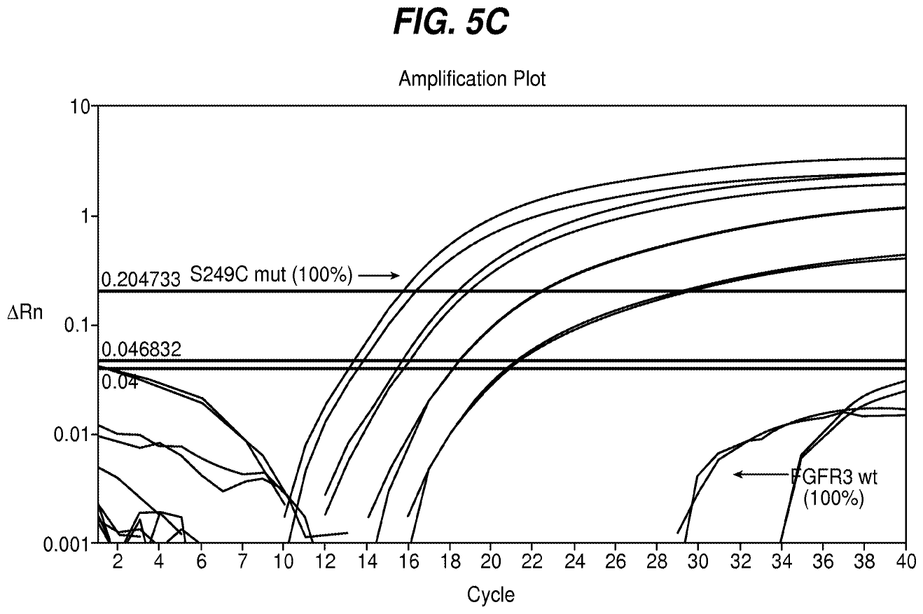

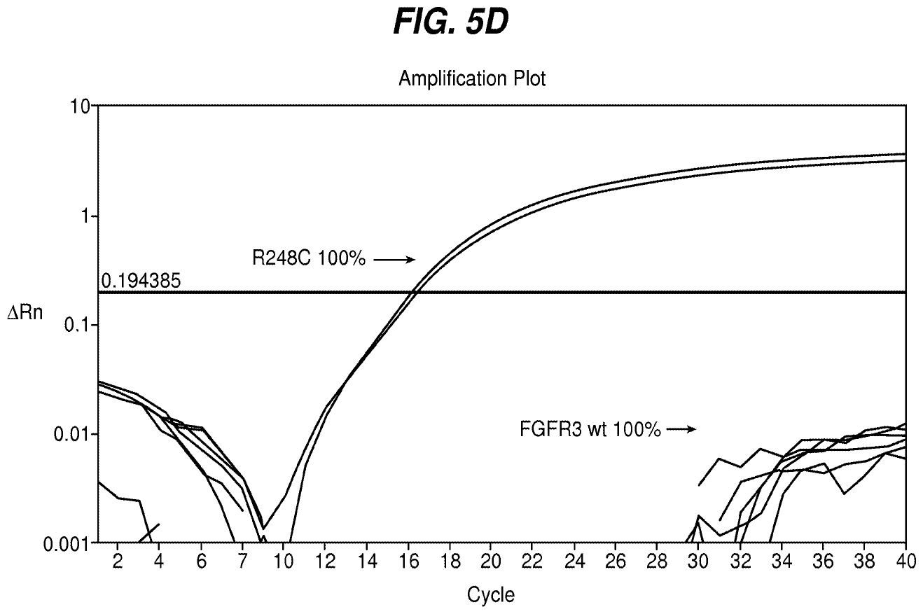

Samples for validatation studies were prepared as shown in Table 9. Exemplary validation data of the SNP-specific qRT-PCR using a 3′ dideoxy WT blocker oligonucleotide for FGFR3 G370C, FGFR3 Y373, FGFR3 S249C, and FGFR3 R248C is illustrated in FIGS. 5A-5D, respectively. Raw Ct (cycle threshold) data for the FFPE samples with SNP-specific qRT-PCR with 3′ dideoxy WT blocker oligonucleotides are shown in Table 10. The data derived from DNA and RNA using different platforms/techniques suggests that SNP-specific PCR with the 3′ blocking nucleotide is a robust, reliable and a sensitive assay. The validation data suggests that one mutant allele/SNP can be detected in a large excess of WT-bearing genomic DNA, thus emphasizing the sensitivity and the specificity of each assay.

TABLE 9

| Sample | % Mutant |
|---|---|
| 1 | 100 |
| 2 | 20 |
| 3 | 4 |
| 4 | 0.8 |
| 5 | 0 (100% WT) |

RNA from Stable cell lines expressing each FGFR3 SNPs (R248C, S249C, G370C, Y373C) and FGFR3 WT

TABLE 10

| | FGFR3 SNPs - SNP-Specific PCR with | | | | | Janssen |
|---|---|---|---|---|---|---|
| | Dideoxy WT Blocker (Ct *) | | | | | R&D |
| Pt Id# | R248C | S249C | G370C | Y373C | FMI/NGS | ver1.0 |
| 7502 | >35 | 28.03 | >35 | >35 | S249C | S249C |
| 10000305 | >35 | >35 | >35 | >35 | WT | WT |
| 33000127 | >35 | 20.92 | >35 | >35 | S249C | S249C |
| 33000118 | >35 | 29.35 | >35 | >35 | S249C | S249C |
| 10000306 | >35 | >35 | >35 | 24.30 | Y373C | Y373C |
| 34000226 | >35 | >35 | >35 | >35 | WT | WT |
| 16446 | >35 | 28.03 | >35 | >35 | S249C | S249C |

* Mean of two Cts
FMI/NGS = Next generation Sequencing technique wherein DNA is used as an template to identify the mutations (without 3′ blocking oligonucleotide); Janssen R&D = performed on RNA template (without the 3′ blocking oligonucleotide); SNP-specific PCR performed on RNA template with the 3′ blocking nucleotide.

### Example 5—Validation of Custom FGFR Fusion Gene Detection Assay

### Generation of Positive Controls for FGFR Fusion Assays

FGFR fusion “synthetic mini-genes,” plasmids encoding FGFR fusions, and stable cell lines containing FGFR fusions were generated. Briefly, Synthetic mini genes were artificially constructed by linking a series of nucleotides, of about 100 base pairs, to each other corresponding to the target DNA sequence of the gene of interest. Plasmids encoding FGFR fusions were generated by cloning cDNA encoding the various FGFR fusion genes into an expression vector. Stable cell lines containing FGFR fusions were generated by transfecting plasmids encoding FGFR genes into normal rat kidney epithelial cells (NRK cells). The stable cell lines were selected under the G418 antibiotic. The FGFR fusion Taqman assay was performed on the total RNA isolated from these cell lines to confirm the successful generation of stable cell line(s) expressing the FGFR fusion(s). The stable cell lines expressing FGFR fusions are used a positive control. Table 15 lists the sequences of the probes used during the real time PCR assays.

### Analysis of Lower Limit of Quantitation and Efficiency of FGFR Fusion Assays

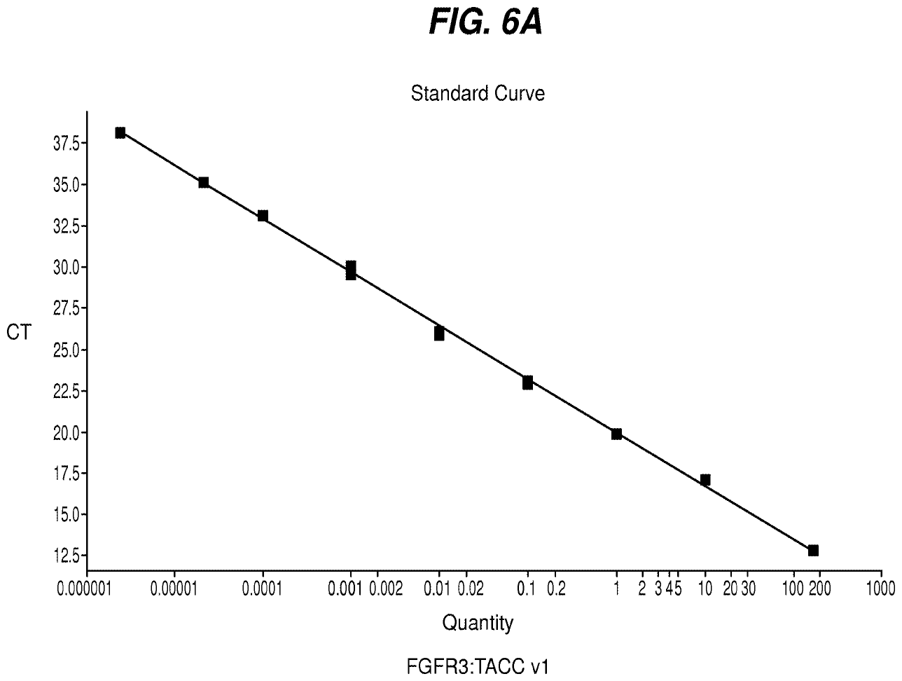

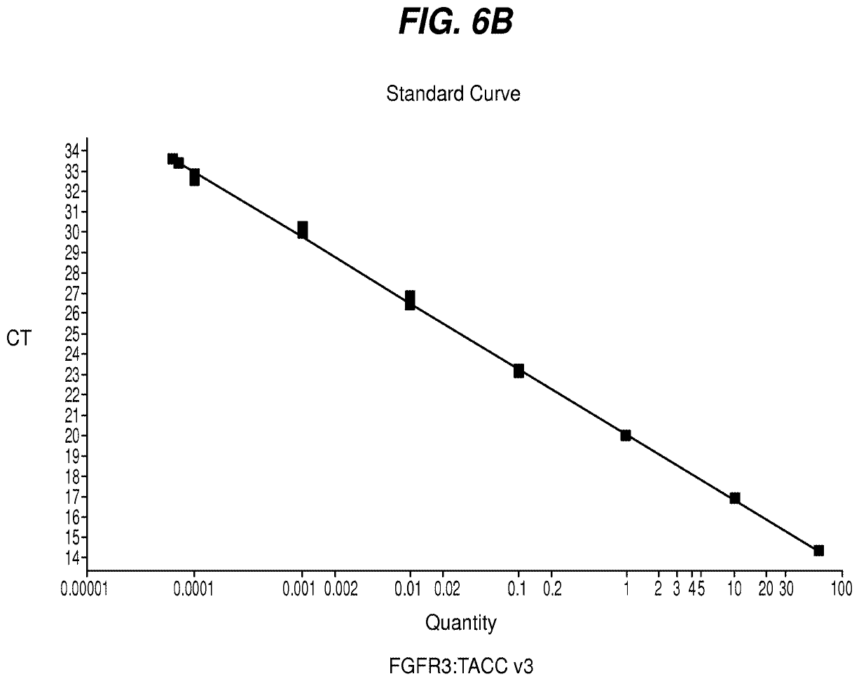

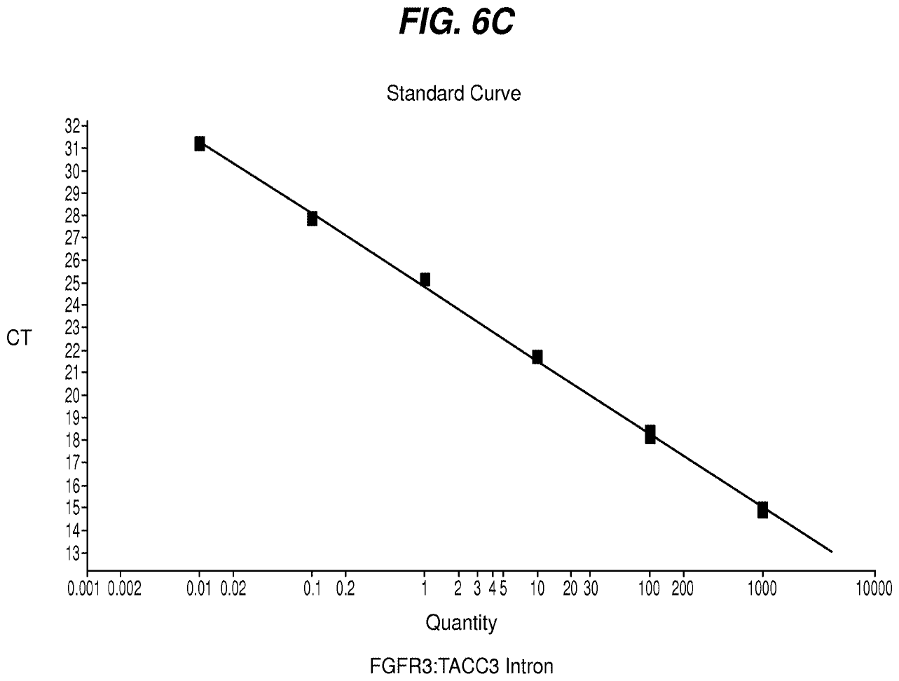

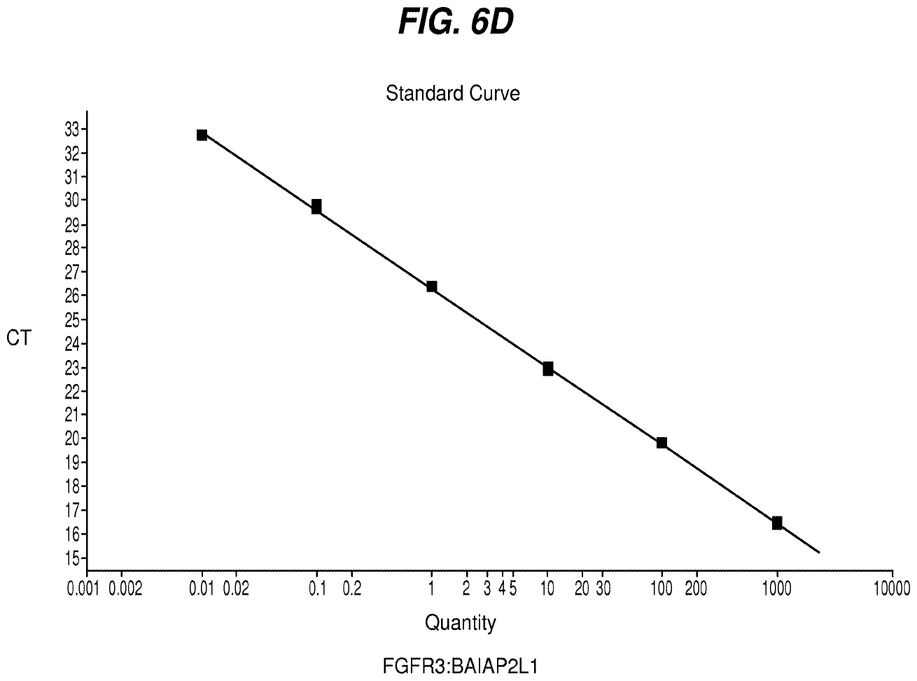

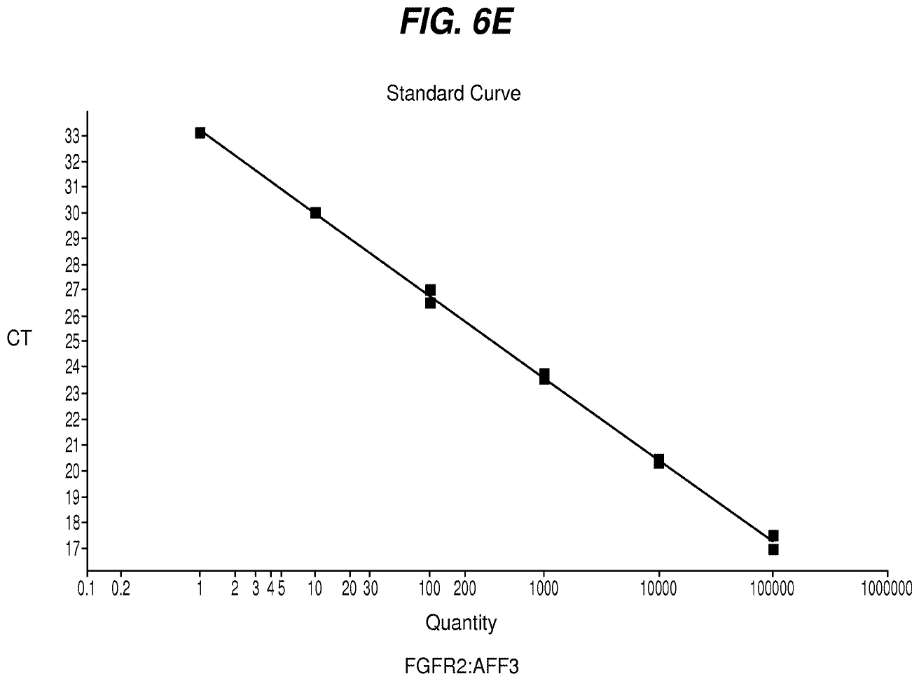

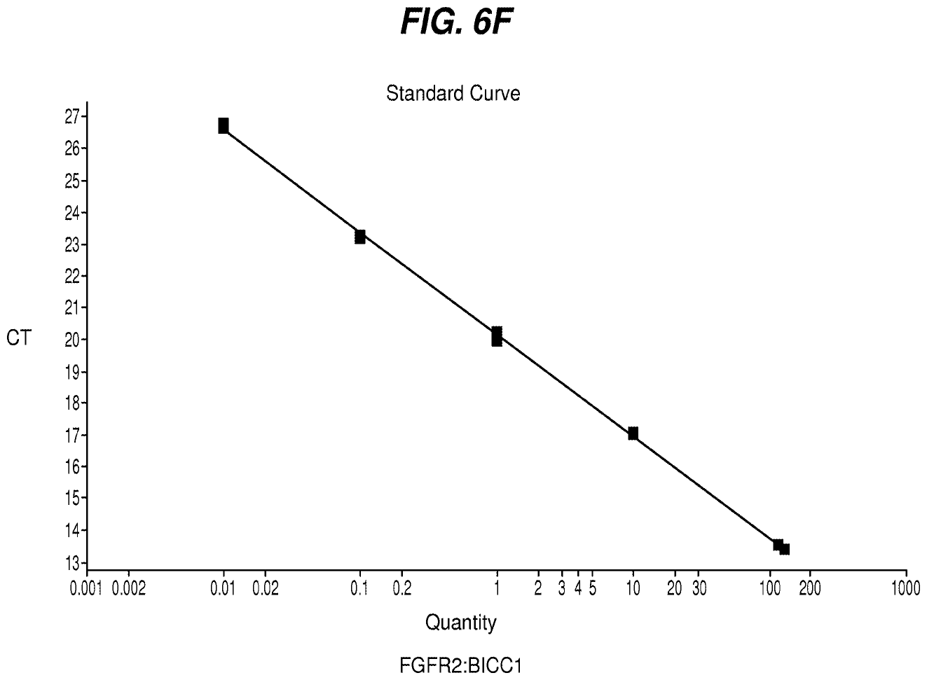

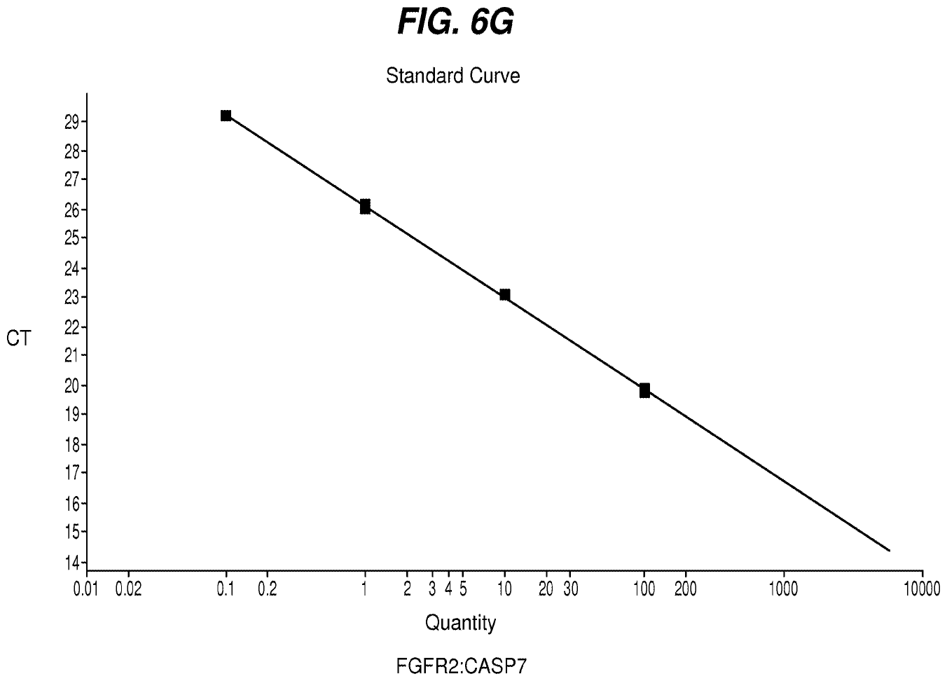

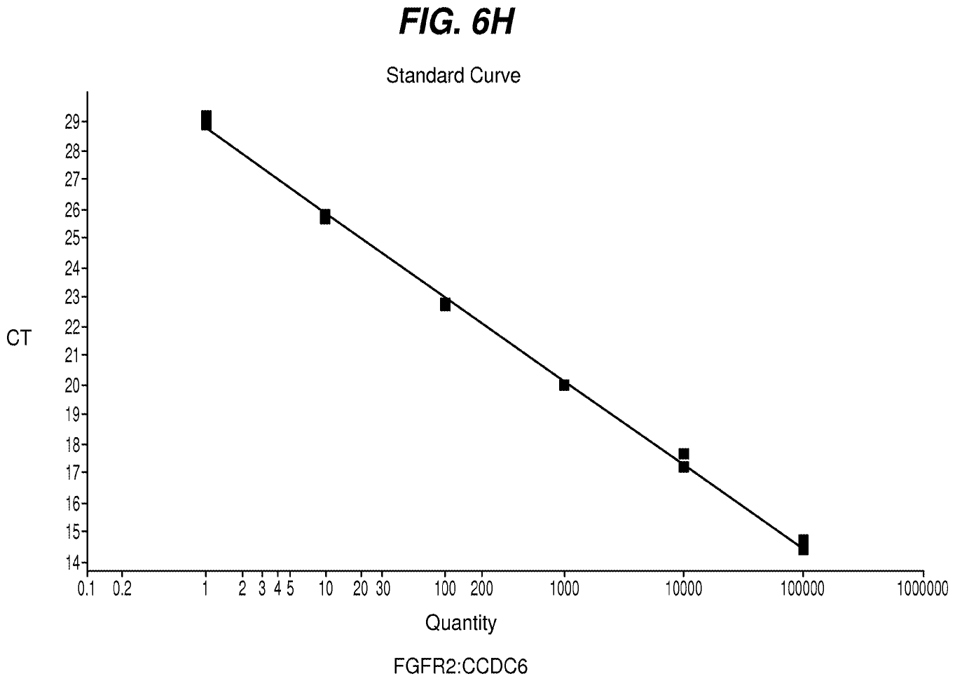

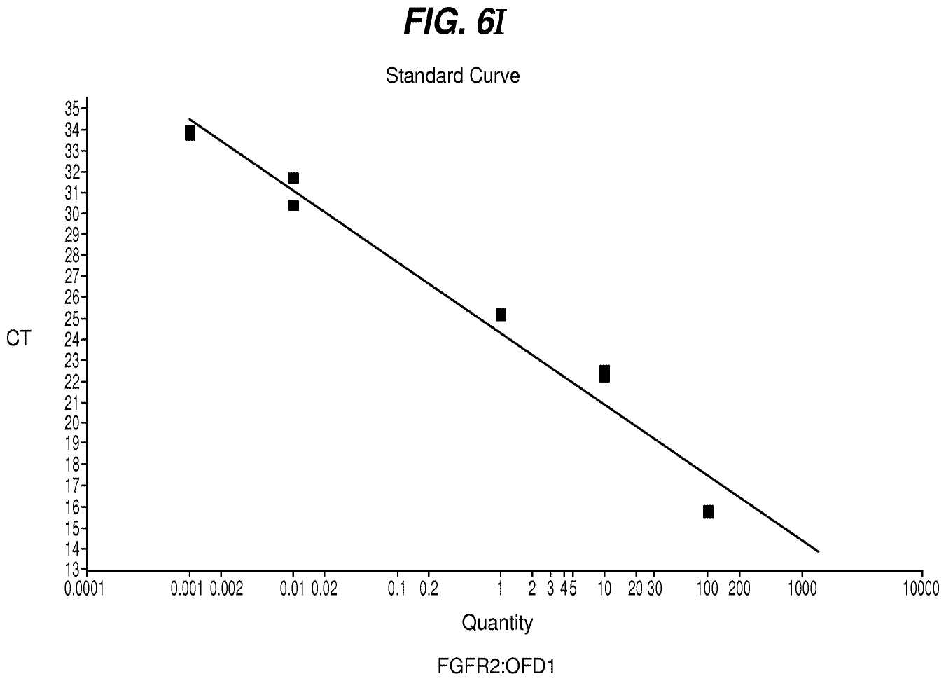

To determine the lower limit of quantitation (LLOQ) and efficiency of the FGFR fusion gene assays, FGFR fusion products were generated by TaqMan PCR (as described in Example 4) and confirmed by Sanger Sequencing (FIGS. 2A-2I). 100 pg of fusion positive DNA was mixed with normal human cDNA (confirmed fusion-negative), serially diluted 1:10, and analyzed using the Applied Biosystems ViiA7 Software v1.1. Efficiency standard curves are shown in FIGS. 6A-6I. FGFR fusion LLOQ and efficiency are shown in Table 11.

TABLE 11

| | | |
|---|---|---|
| Assay | LLOQ | Efficiency |

| | | | |
|---|---|---|---|
| FGFR3:TACC3 V1 | 1.0 | fgm | 104% |
| FGFR3:TACC3 V3 | 10.0 | fgm | 104% |
| FGFR3:TACC3 Intron | 0.1 | fgm | 103% |
| FGFR3:BAIAP2L1 | 1.0 | fgm | 101% |
| FGFR2:AFF3 | 0.1 | fgm | 106% |
| FGFR2:BICC1 | 10.0 | fgm | 105% |
| FGFR2:CASP7 | 0.1 | fgm | 109% |
| FGFR2:CCDC6 | 1.0 | fgm | 106% |
| FGFR2:OFD1 | 0.1 | fgm | 96.6% |

The FGFR fusion gene assay was next validated in fusion gene-positive cell lines. FGFR fusion gene expression, serial dilutions were prepared by spiking fusion protein-positive cells lines into a fusion protein-negative cell line. For example, a 1:2 serial dilution was prepared for both FGFR3:TACC3v1 and FGFR3:BAIAP2L1 and spiked into 1 million BAF cells. RNA was isolated (using Qiagen Rneasy kit), followed by RT-PCR, preamplification of cDNA, and TaqMan Real Time PCR for the targeted FGFR fusion gene. As shown in Table 12, both the FGFR3:TACC3v1 and FGFR3:BAIAP2L1 Fusion Gene TaqMan assays are able to detect the fusion target in 31 out of 1 million fusion-negative cells (sensitivity of 0.003%).

TABLE 12

| | | Percent of | RT112 | SW780 |
|---|---|---|---|---|
| | | Fusion-Positive | FGFR3:TACC3v1 | FGFR3:BAIAP2L1 |
| | FGFR-fusion | Cells vs | Average Ct | Average Ct |
| | Cell Count | Background | (n = 2) | (n = 2) |
| Positive | 1.00E+06 | 100% | 17.56 | 20.35 |
| Control | | | | |
| | 1000 | 0.1000% | 27.95 | 28.61 |
| | 500 | 0.0500% | 29.11 | 28.91 |
| | 250 | 0.0250% | 29.62 | 30.14 |
| | 125 | 0.0125% | 30.26 | 31.43 |
| | 62.5 | 0.0063% | 31.19 | 31.69 |
| LLOD | 31.25 | 0.0031% | 32.59 | 32.97 |
| | 15.6 | 0.0016% | 34.91 | >40 |
| | 0 | 0.0000% | 0.00 | >40 |

RT112 and SW780 = commercially available bladder cancer cell lines harboring the FGFR fusions (from American Type Culture Collection).

### Example 6—Validation of Custom FGFR SNP Detection Assay

### Evaluation of FGFR3 Mutations in Bladder Cancer

The R248C, S249C, and Y373C SNPs were observed in approximately 8%, approximately 61%, and approximately 19% of bladder cancer samples tested, respectively.

### Example 7—Analysis of Cancer Samples

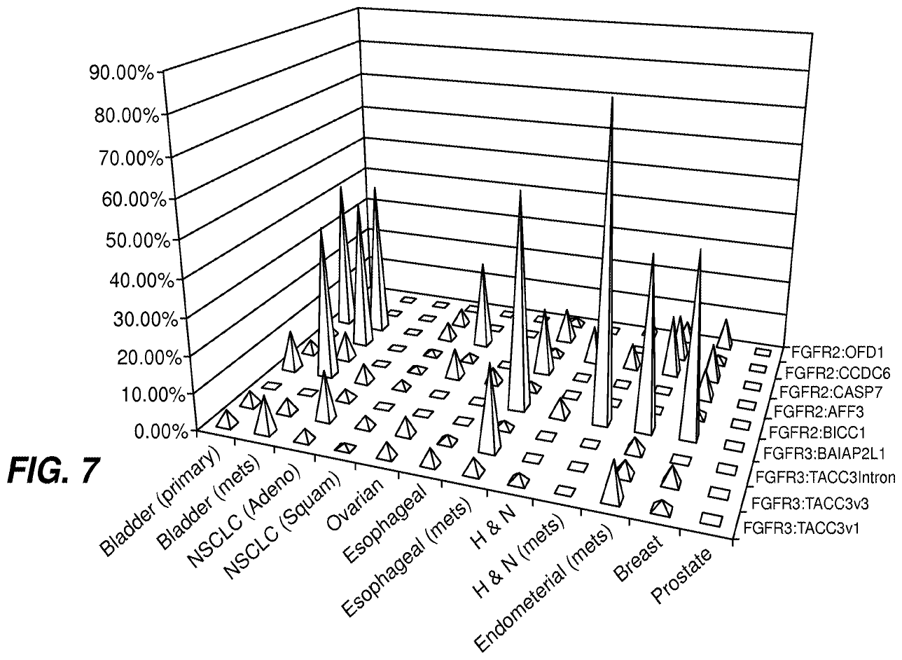

Samples were analyzed using the same procedure as described in example 4. The results are shown in Table 13 and FIG. 7. Table 13 shows the FGFR fusion prevalence in different cancers. FGFR fusions detected in FFPE samples from different cancers such as bladder (primary and metastatic), NSCLC (adenocarcinoma and squamous), ovarian, esophageal (primary and metastatic), head and neck (H&N; primary and metastatic), endometrial (metastatic), breast, and prostate cancer using the qRT-PCR method. All FGFR fusions tested were negative for prostate cancer samples. FGFR3:TACC3intron fusion was negative in bladder (primary), NSCLC (squamous), ovarian and esophageal (primary), H&N (primary and metastatic) and breast. FGFR2:OFD1 fusion was negative in bladder (primary and metastatic), NSCLC (adenocarcinoma), ovarian and esophageal (primary and metastatic). FGFR2:CCDC6 fusion was negative in bladder (primary and metastatic), NSCLC (adenocarcinoma), ovarian and esophageal (primary) and H&N (primary and metastatic)

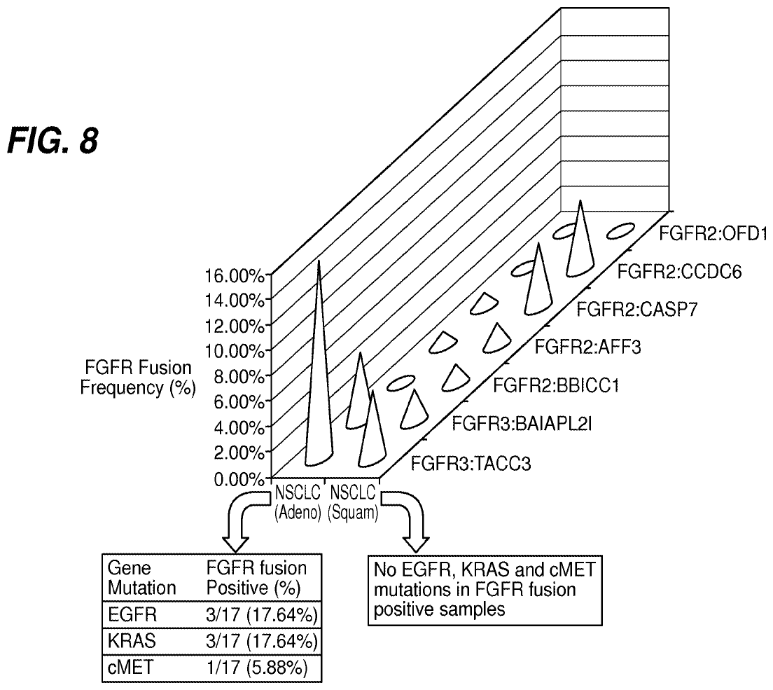

FIG. 8 is an exemplary representation of FGFR fusion gene and mutation status in NSCLC adenocarcinoma and squamous cell carcinoma. In FGFR fusion positive NSCLC adenocarcinoma samples, 3/17 samples were positive for EGFR mutation, 3/17 samples were positive for KRAS mutation, and 1/17 samples were positive for cMET mutation. No EGFR, KRAS, or cMET mutations, however, were observed in FGFR fusion positive NSCLC squamous cell carcinoma samples.

TABLE 13

| | Bladder | Bladder | NSCLC | NSCLC | | Eso |
|---|---|---|---|---|---|---|
| | primary (%) | Mets (%) | Adeno (%) | Squamous (%) | Ovarian (%) | Primary (%) |
| FGFR3: | 1/22 | 5/48 | 3/89 | 2/125 | 4/94 | 2/41 |
| TACC3v1 | (4.55) | (10.47) | (3.37) | (1.60) | (4.26) | (4.88) |
| FGFR3: | 1/22 | 2/48 | 9/89 | 5/129 | 5/94 | 1/41 |
| TACC3v3 | (4.55) | (4.20) | (13.90) | (3.38) | (5.32) | (2.44) |
| FGFR3: | 0/22 | 0/48 | 3/89 | 0/125 | 0/94 | 0/41 |
| TACC3Intron | (0.00) | (0.00) | (3.37) | (0.00) | (0.00) | (0.00) |
| FGFR3: | 2/17 | 19/44 | 5/89 | 3/115 | 1/94 | 0/41 |
| BAIAP2L1 | (11.77) | (43.18) | (5.62) | (2.61) | (1.06) | (0.00) |
| FGFR2: | 1/22 | 4/48 | 0/89 | 2/123 | 8/94 | 2/41 |
| BICC1 | (4.55) | (8.33) | (0.00) | (1.63) | (8.51) | (4.88) |
| FGFR2: | 1/17 | 19/44 | 1/89 | 2/111 | 2/94 | 0/41 |
| AFF3 | (5.88) | (43.18) | (1.12) | (1.80) | (2.31) | (0.00) |
| FGFR2: | 7/16 | 20/45 | 1/89 | 6/114 | 24/94 | 2/41 |
| CASP7 | (43.75) | (44.44) | (1.12) | (5.26) | (25.53) | (4.88) |
| FGFR2: | 0/22 | 0/48 | 0/89 | 6/109 | 0/94 | 0/41 |
| CCDC6 | (0.00) | (0.00) | (0.00) | (5.50) | (0.00) | (0.00) |
| FGFR2: | 0/17 | 0/44 | 0/89 | 1/121 | 0/94 | 0/41 |
| OFD1 | (0.00) | (0.00) | (0.00) | (0.83) | (0.00) | (0.00) |

| | Eso | H&N | H&N | Endo | | |
|---|---|---|---|---|---|---|
| | Mets (%) | Primary (%) | Mets (%) | Mets (%) | Breast (%) | Prostate (%) |
| FGFR3: | 2/42 | 1/37 | 0/40 | 5/46 | 3/112 | 0/72 |
| TACC3v1 | (4.76) | (2.70) | (0.00) | (10.87) | (2.69) | (0.00) |
| FGFR3: | 10/42 | 0/37 | 0/40 | 2/46 | 6/112 | 0/72 |
| TACC3v3 | (23.81) | (0.00) | (0.00) | (4.35) | (5.36) | (0.00) |
| FGFR3: | 1/42 | 0/37 | 0/40 | 2/46 | 0/112 | 0/72 |
| TACC3Intron | (2.38) | (0.00) | (0.00) | (4.35) | (0.00) | (0.00) |
| FGFR3: | 25/42 | 2/37 | 34/40 | 22/46 | 56/112 | 0/72 |
| BAIAP2L1 | (59.52) | (5.41) | (85.00) | (47.83) | (50.00) | (0.00) |
| FGFR2: | 1/42 | 0/37 | 0/40 | 0/46 | 3/112 | 0/72 |
| BICC1 | (2.40) | (0.00) | (0.00) | (0.00) | (2.70) | (0.00) |
| FGFR2: | 8/42 | 0/37 | 0/40 | 0/46 | 10/112 | 0/72 |
| AFF3 | (19.05) | (0.00) | (0.00) | (0.00) | (8.90) | (0.00) |
| FGFR2: | 1/42 | 4/37 | 3/40 | 8/46 | 12/112 | 0/72 |
| CASP7 | (2.40) | (10.81) | (7.50) | (17.40) | (10.70) | (0.00) |
| FGFR2: | 4/42 | 0/37 | 0/40 | 6/46 | 3/112 | 0/72 |
| CCDC6 | (9.52) | (0.00) | (0.00) | (13.04) | (2.70) | (0.00) |
| FGFR2: | 1/42 | 0/37 | 3/40 | 3/46 | 10/112 | 0/72 |
| OFD1 | (2.40) | (0.00) | (7.50) | (6.52) | (8.90) | (0.00) |

Eso = Esophageal;
Endo = Endometerial

### Example 8—Treatment of Patients with Advanced Solid Tumors

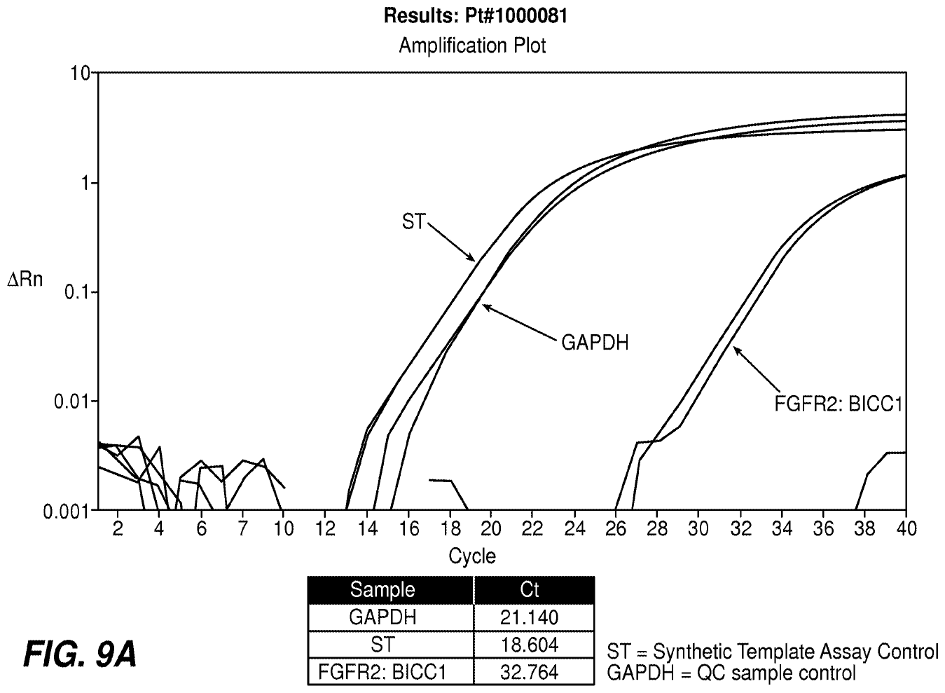

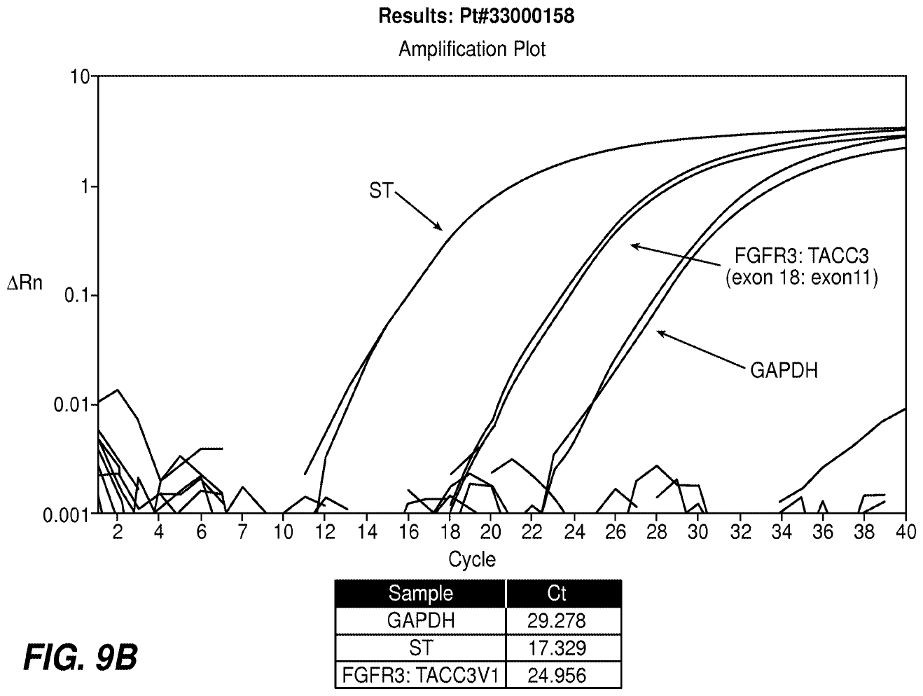

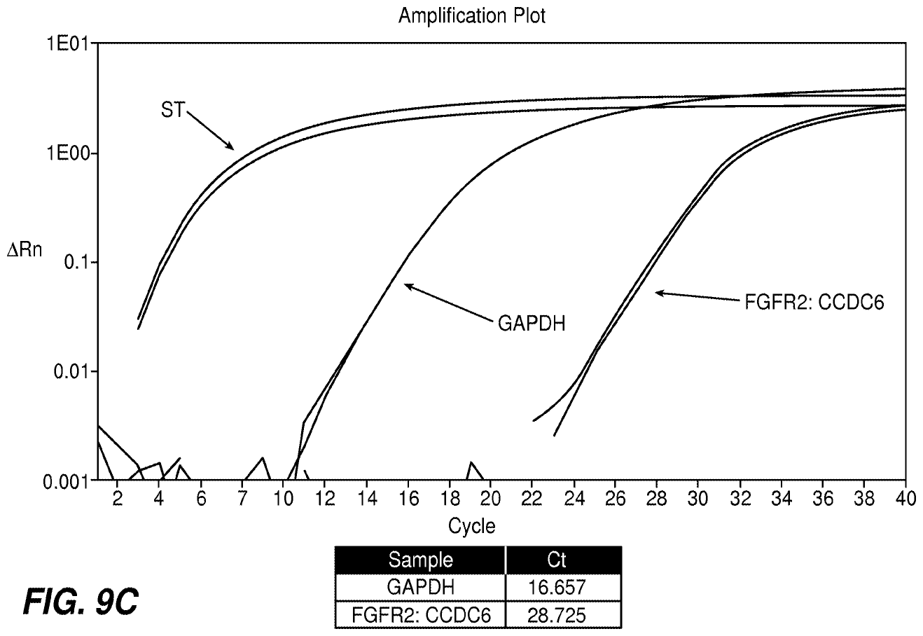

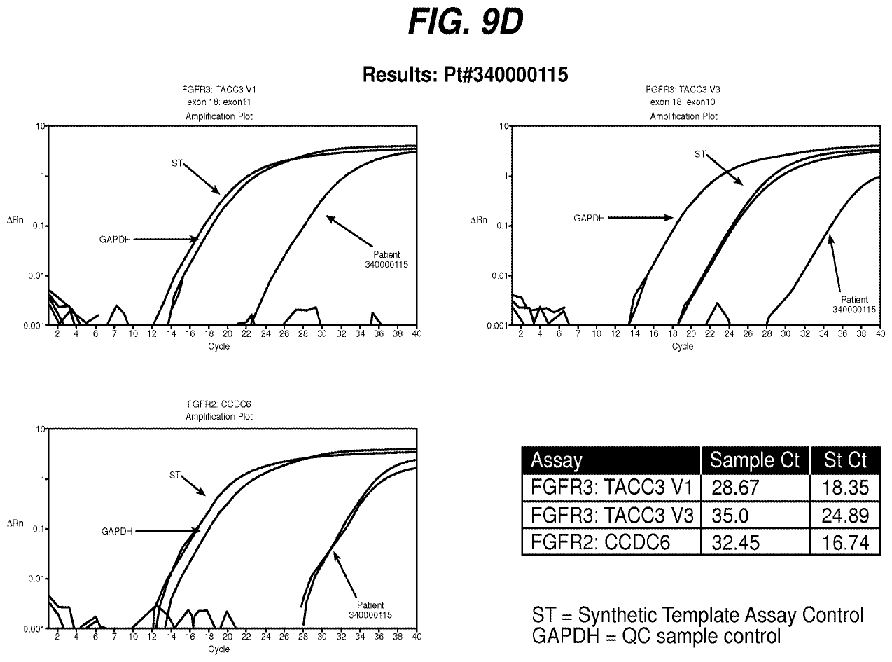

A clinical trial was conducted in which patients having various solid tumors expressing the FGFR3:TACC3 v1, FGFR3:TACC3 v3, FGFR2:CCDC6 and FGFR2:BICC1 fusion genes were treated with JNJ-42756493. FIGS. 9A-9D illustrates exemplary results from phase I patient samples, in which FGFR fusions in Phase I JNJ-427493 (EDI10001) trial samples were detected using the qRT-PCR assay. All FGFR fusion assays were run simultaneously with positive controls (ST) and GAPDH for quality control assessment of the RNA. FIG. 9A) Graphical representation of the qRT-PCR data generated for pt #1000081: positive only for FGFR2:BICC1 fusion (inset shows details of the Ct values for FGFR2:BICC1 fusion, ST-positive control and GAPDH). FIG. 9B) Graphical representation of the qRT-PCR data generated for pt #33000158: positive only for FGFR3:TACC3v1 fusion (inset shows details of the Ct values for FGFR3:TACC3v1 fusion, ST-positive control and GAPDH). FIG. 9C) Graphical representation of the qRT-PCR data generated for pt #34000123: positive only for FGFR2:CCDC6 fusion (inset shows details of the Ct values for FGFR2:CCDC6 fusion, ST-positive control and GAPDH). FIG. 9D) Graphical representation of the qRT-PCR data generated for pt #340000115: positive for FGFR3:TACC3v1, FGFR3:TACC # v3 and FGFR2:CCDC6 fusions (inset shows details of the Ct values for FGFR fusions, ST-positive controls and GAPDH).

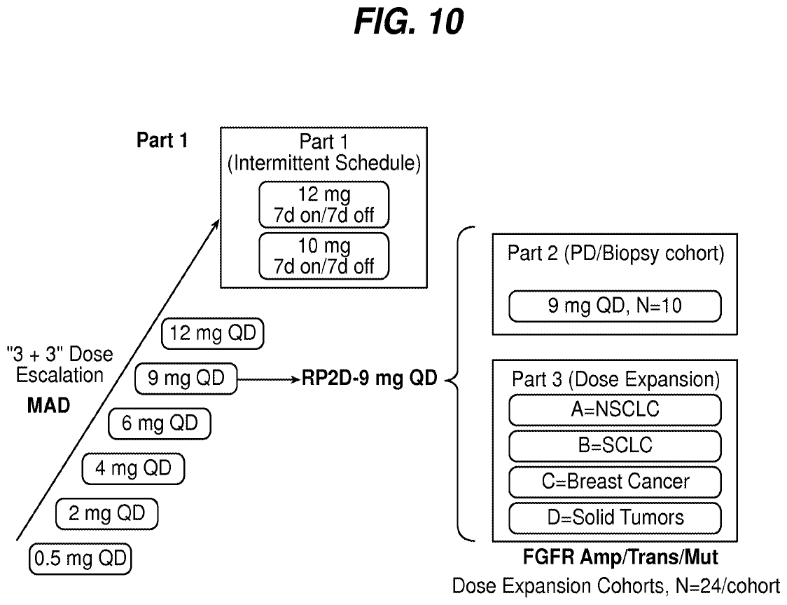

FIG. 10 represents an exemplary Phase I Study design for a First-In-Human Study of JNJ-42756493 in patients with advanced solid tumor. Shown is a graphical depiction of a traditional 3+3 design dose escalation method for the phase I clinical trial. The dose escalation phase aimed to establish the maximum tolerated dose (MTD) and recommended Phase II dose (RPD). The Part 1 arm was used to determine the intermittent dosing schedule, i.e., 7 days on and seven days off (10 mg/kg and 12 mg/kg). The Part 2 arm was used to determine the PD biomarkers (pharmacodynamics biomarkers; makers examined to link the effect of the drug to the target and biological tumor response) wherein the biopsy and blood sample were tested. The Part 3 arm was used the dose expansion cohort and included accrual of additional patients in specific indications (NSCLC,SCLC, breast and solid tumors) with different eligibility criteria (FGFR aberrations: translocation/mutation/amplifications) to further characterize the toxicity profiles of the JNJ493.

### Evaluation of Clinical Activity

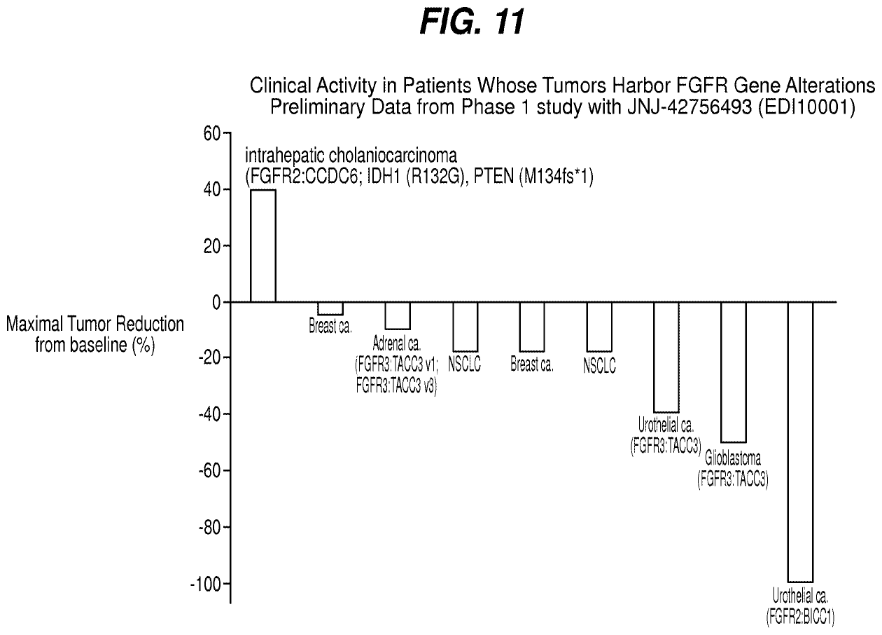

Significant clinical responses (RECIST) were observed at 9 mg dosing once a day (QD), 12 mg QD and 12 mg 7d on/off in patients with the FGFR fusion genes. (FIG. 11; represents all dosing regimens).

### Example 9—Generation of FGFR Fusion Stably Transfected RK3E Cells

### FGFR Fusion Overexpressing Cell Lines

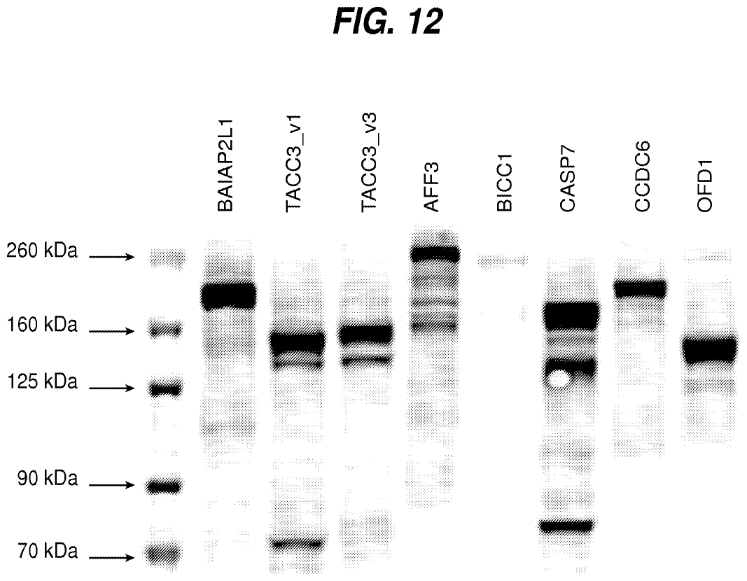

RK3E (rat kidney epithelial cells) cells were purchased from ATCC (Manassas, Va., USA) and cultured in DMEM supplemented with FBS and antibiotics (Invitrogen, Grand Island, N.Y., USA). FGFR fusion gene constructs were designed and cloned into the pReceiver expression vector (Genecopoeia, Rockville, Md., USA), which contains an HA-tag. Clones were transfected into RK3E cells using the Amaxa Cell Line Nucleofector (Lonza, Basel, Switzerland) following the manufacturer's protocol. The stably transfected cells were selected in complete medium with 800 ug/ml of G418 (Invitrogen). Overexpression of the fusions in the stably transfected cells was confirmed by real-time PCR and immunoblotting using an anti-pFGFR antibody (FIG. 12). As shown in FIG. 12, the stable cell lines showed expression of active FGFR fusion kinases, as exhibited by the expression of phosphorylation of FGFR.

### Colony Formation Assay

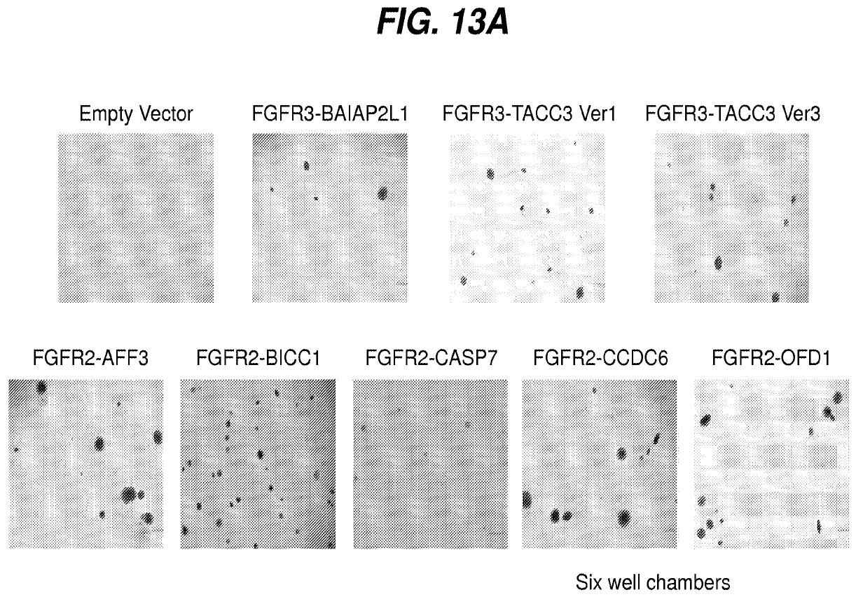

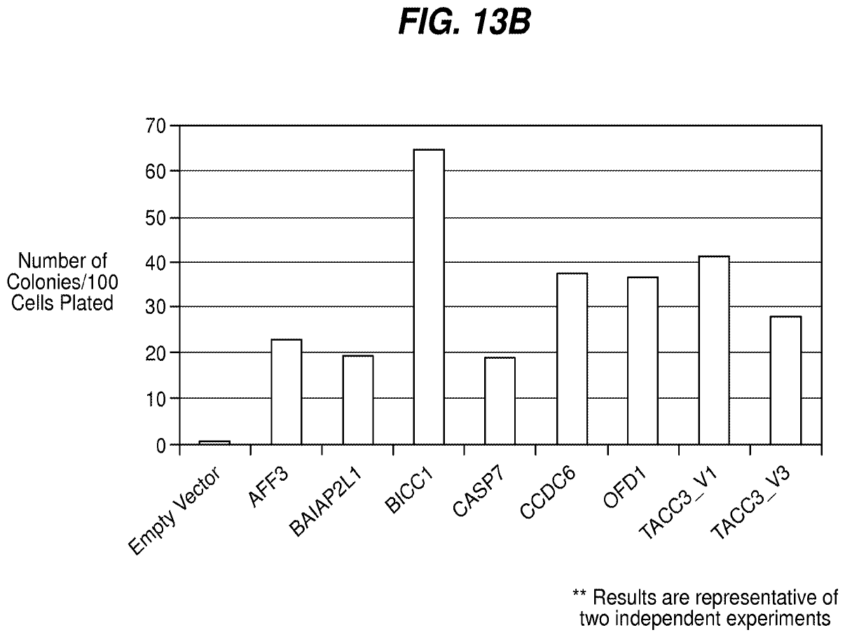

Anchorage-independent growth of the FGFR fusion stably transfected RK3E cells was tested. 1 ml culture medium with 0.8% low melting point agarose was first plated into each of three wells of a six-well plate. After the agar solidified, each well received another 1 ml of 0.4% agar in culture medium containing 100 cells. After 14 days, colonies were fixed and stained with 0.1% cresyl crystal violet. The number of colonies was determined microscopically by manual counting from triplicate wells for each cell line. A representative view of each fusion-overexpressing cell line is shown in FIG. 13A. Anchorage-independent cell growth in soft-agar could be detected in the FGFR fusion stably transfected cells, but not in the empty vector control. FIG. 13B represents a quantitative analysis of colonies in soft agar for the FGFR fusion stably transfected RK3E cells and empty vector control. All experiments were carried out in duplicate and the results are expressed as colonies/100 cells plated. All of the FGFR fusions tested induced anchorage independent growth, highlighting their transforming ability

### Downstream Target Expression

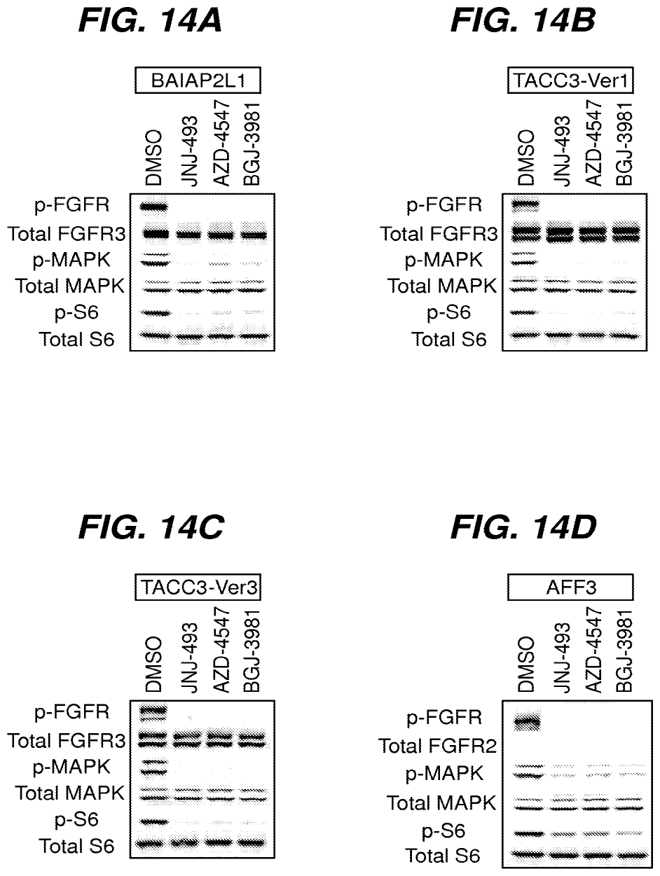

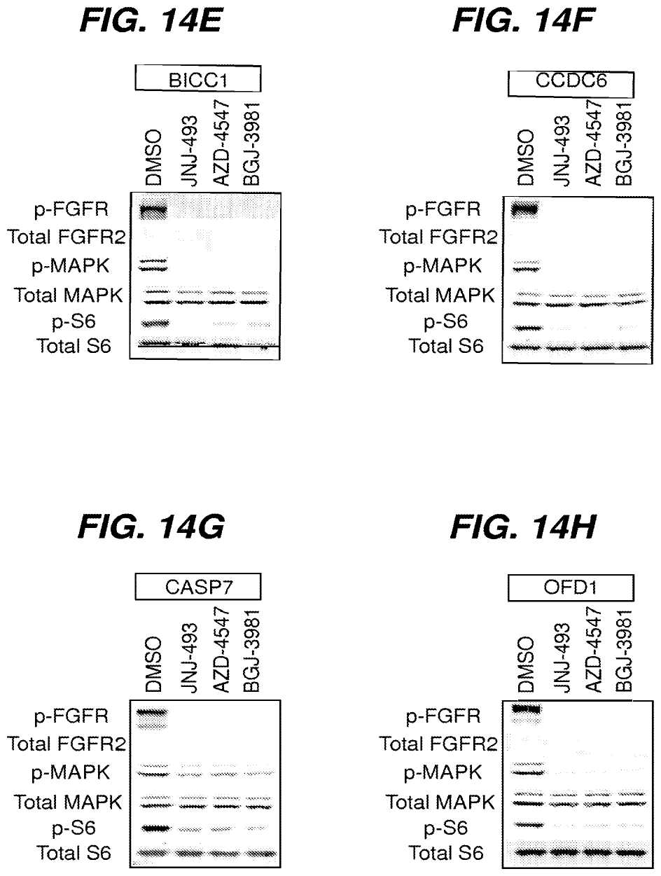

FGFR fusion stably transfected RK3E cells were plated in complete growth medium, serum starved overnight, then re-fed with 0.5% FBS growth media. Cells were treated with 1 μM of JNJ-42756493, AZD4547 or NVP-BGJ398 in the presence of ligands for 1 hour. For immunoblotting, whole cell lysates were collected in RIPA buffer (Thermo Scientific, Waltham, Mass., USA) and sample protein concentration was assayed using BCA Protein Assay (Thermo Scientific). Equal amounts of protein (30 μg per lane) were loaded onto on 4-12% Bis-Tris gels (Invitrogen) before an SDS-page was performed. Proteins were transferred to nitrocellulose membranes and probed with antibodies against p-FGFR, total-FGFR2, p-MAPK, total-MAPK, p-S6, total S6, B-actin (Cell Signaling Technology, Danvers, Mass., USA), and total-FGFR3 (Santa Cruz, Dallas, Tex., USA). The membranes were blocked with Odyssey blocking buffer for 1 h at room temperature and incubated overnight at 4° C. in a primary antibody solution diluted in Odyssey blocking buffer (1:1000). After three washes in 0.1% Tween tris buffered saline (TBST), the membranes were probed with goat anti-mouse or donkey anti-rabbit IR-Dye 670 or 800 cw labeled secondary antisera in Odyssey blocking buffer for 1 h at room temperature. Washes were repeated after secondary labeling and the membranes were imaged using a LiCor Odyssey scanner and the Odyssey 3.0 analytical software (LiCor, Lincoln, Nebr., USA). Effects of JNJ-42756493 was compared with AZD4547 and NVP-BGJ398. As shown in FIGS. 14A-14H, treatment with JNJ-42756493, AZD4547 and NVP-BGJ398 (lanes 2-4 in each blot) inhibited phosphorylation of FGFR and downstream targets i.e. MAPK and S6.

### Drug Response Testing for FGFR Fusion Overexpressing Cell Lines

FGFR fusion stably transfected RK3E cells were seeded into 96 well plates (1000 cells/well) in triplicates in complete growth medium plus and the ligands FGF-1 and FGF-2. After 24 hours, cells were serum starved overnight, then re-fed with 0.5% FBS growth media. 72 hours after plating, cells were treated with various concentrations of an 18 point 1:3 dilution series, starting at 10 μM, of JNJ493, AZD4547 (AZD), and NVP-BGJ398 (NVS). The Microtiter plates were then incubated for 72 hours and assayed for adenosine triphosphate (ATP; a marker of metabolically active cells) content using the Cell Titer-Glo® Luminescent Cell Viability assay (Promega Corp., Madison, Wis., USA) following the manufacturer's instructions, with modifications. Briefly, cells were allowed to equilibrate to room temperature, at which time a 1:1 mixture of Cell Titer-Glo® reagent was added. Cells were then placed on an orbital shaker for 2 minutes and incubated for 10 minutes at room temperature to stabilize the luminescent signal. The luminescence was quantified and measurements were conducted using an Envision Multilabel plate reader (Perkin Elmer; Waltham, Mass., USA). IC₅₀ values (shown in Table 14) were calculated using GraphPad Prism 5.0. As shown in Table 14, cells harboring the FGFR fusions showed sensitivity to the FGFR inhibitor JNJ-42756493, AZD4547 and NVP-BGJ398 in vitro, with JNJ-42756493 exhibiting enhanced sensitivity (nanomolar concentration range) when compared to AZD4547 and NVP-BGJ398, whereas the empty vector control did not.

TABLE 14

| Stimulated | Proliferation (IC50) | | |
|---|---|---|---|
| RK3E-Transgene | JNJ493 (nM) | AZD (nM) | NVS (nM) |
| Vector | 7010 | 8011 | >10 μM |
| AFF3 | 0.1133 | 2.809 | 2.273 |
| BAIA2PL1 | 0.3211 | 11.54 | 5.162 |
| BICC1 | 0.3303 | 6.448 | 18.19 |
| CASP7 | 0.4718 | 4.107 | 241.5 |
| CCDC6 | 0.1894 | 13.36 | 10.72 |
| OFD1 | 0.2303 | 7.259 | 15.99 |
| TACC3-V1 | 0.2915 | 16.53 | 2.594 |
| TACC3-V3 | 0.2706 | 8.664 | 4.092 |
| FGFR2 | >10 μM | 6501 | >10 μM |
| FGFR3 | >10 μM | 5686 | 6344 |
| KRAS | 1621 | 1478 | 2136 |

AZD = AZD4547;
NVS = NVP-BGJ398

TABLE 15

| Target | Probe Sequences |
|---|---|
| FGFR3TACC3 V1 | TCCACCGACGTAAAGG |
| | (SEQ ID NO: 43) |
| FGFR3TACC3 V3 | TCCACCGACGTGCCAG |
| | (SEQ ID NO: 44) |
| FGFR2BICC1 | CCAATGAGATCATGGAGG |
| | (SEQ ID NO: 45) |
| FGFR3TACC3 Intron | CCTTCTGGCCCAGGTG |
| | (SEQ ID NO: 46) |
| FGFR3BAIAP2L1 | CACCGACAATGTTATGG |
| | (SEQ ID NO: 47) |
| FGFR2AFF3 | TCACAACCAATGAGGAGAGT |
| | (SEQ ID NO: 48) |
| FGFR2CASP7 | CTGCCATCTCATTGGT |
| | (SEQ ID NO: 49) |
| FGFR2CCDC6 | AATGAGCAAGCCAGGGC |
| | (SEQ ID NO: 50) |
| FGFR2OFD1 | AAGTTGTGTCTCATTGGTT |
| | (SEQ ID NO: 51) |
| FGFR3 R248C | CTGGAGTGCTCCCC |
| | (SEQ ID NO: 52) |
| FGFR3 S249C | AGCGCTGCCCGCA |
| | (SEQ ID NO: 53) |
| FGFR3 G370C | GCGTGCAGTGTGTAT |
| | (SEQ ID NO: 54) |
| FGFR3 Y373 | CTGCACACACACTGC |
| | (SEQ ID NO: 55) |

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### Nucleotide Sequence of FGFR Fusion Genes

The nucleotide sequences for the FGFR fusion cDNA that were engineered into expression vectors is provided in Table 16. The underlined sequences correspond to either FGFR3 or FGFR2, the sequences in normal font represent the fusion partners and the sequence in italic fonts represent the intron sequence of the FGFR3 gene.

TABLE 16

| | |
|---|---|
| FGFR3:TACC3 v1 | >ATGGGCGCCCCTGCCTGCGCCCTCGCGCTCTGCGTGGCCGTGGCCATCGTGGCCGGCGCCTCCTCGGAGTCCTTGGGGACGGAGCA |
| (3271 base | GCGCGTCGTGGGGCGAGCGGCAGAAGTCCCGGGCCCAGAGCCCGGCCAGCAGGAGCAGTTGGTCTTCGGCAGCGGGGATGCTGTG |
| pairs) | GAGCTGAGCTGTCCCCCGCCCGGGGGTGGTCCCATGGGGCCCACTGTCTGGGTCAAGGATGGCACAGGGCTGGTGCCCTCGGAGCG |
| (SEQ ID NO: 56) | TGTCCTGGTGGGGCCCCAGCGGCTGCAGGTGCTGAATGCCTCCCACGAGGACTCCGGGGCCTACAGCTGCCGGCAGCGGCTCACGC |
| | AGCGCGTACTGTGCCACTTCAGTGTGCGGGTGACAGACGCTCCATCCTCGGGAGATGACGAAGACGGGGAGGACGAGGCTGAGGA |
| | CACAGGTGTGGACACAGGGGCCCCTTACTGGACACGGCCCGAGCGGATGGACAAGAAGCTGCTGGCCGTGCCGGCCGCCAACACC |
| | GTCCGCTTCCGCTGCCCAGCCGCTGGCAACCCCACTCCCTCCATCTCCTGGCTGAAGAACGGCAGGGAGTTCCGCGGCGAGCACCG |
| | CATTGGAGGCATCAAGCTGCGGCATCAGCAGTGGAGCCTGGTCATGGAAAGCGTGGTGCCCTCGGACCGCGGCAACTACACCTGCG |
| | TCGTGGAGAACAAGTTTGGCAGCATCCGGCAGACGTACACGCTGGACGTGCTGGAGCGCTCCCCGCACCGGCCCATCCTGCAGGCG |
| | GGGCTGCCGGCCAACCAGACGGCGGTGCTGGGCAGCGACGTGGAGTTCCACTGCAAGGTGTACAGTGACGCACAGCCCCACATCC |
| | AGTGGCTCAAGCACGTGGAGGTGAATGGCAGCAAGGTGGGCCCGGACGGCACACCCTACGTTACCGTGCTCAAGACGGCGGGCGC |
| | TAACACCACCGACAAGGAGCTAGAGGTTCTCTCCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGAGGAGCTGGTGGAGGCTGACGAGGCGGGCAGT |
| | GTGTATGCAGGCATCCTCAGCTACGGGGTGGGCTTCTTCCTGTTCATCCTGGTGGTGGCGGCTGTGACGCTCTGCCGCCTGCGCAGC |
| | CCCCCCAAGAAAGGCCTGGGCTCCCCCACCGTGCACAAGATCTCCCGCTTCCCGCTCAAGCGACAGGTGTCCCTGGAGTCCAACGC |
| | GTCCATGAGCTCCAACACACCACTGGTGCGCATCGCAAGGCTGTCCTCAGGGGAGGGCCCCACGCTGGCCAATGTCTCCGAGCTCG |
| | AGCTGCCTGCCGACCCCAAATGGGAGCTGTCTCGGGCCCGGCTGACCCTGGGCAAGCCCCTTGGGGAGGGCTGCTTCGGCCAGGTG |
| | GTCATGGCGGAGGCCATCGGCATTGACAAGGACCGGGCCGCCAAGCCTGTCACCGTAGCCGTGAAGATGCTGAAAGACGATGCCA |
| | CTGACAAGGACCTGTCGGACCTGGTGTCTGAGATGGAGATGATGAAGATGATCGGGAAACACAAAAACATCATCAACCTGCTGGG |
| | CGCCTGCACGCAGGGCGGGCCCCTGTACGTGCTGGTGGAGTACGCGGCCAAGGGTAACCTGCGGGAGTTTCTGCGGGCGCGGCGG |
| | CCCCCGGGCCTGGACTACTCCTTCGACACCTGCAAGCCGCCCGAGGAGCAGCTCACCTTCAAGGACCTGGTGTCCTGTGCCTACCA |
| | GGTGGCCCGGGGCATGGAGTACTTGGCCTCCCAGAAGTGCATCCACAGGGACCTGGCTGCCCGCAATGTGCTGGTGACCGAGGACA |
| | ACGTGATGAAGATCGCAGACTTCGGGCTGGCCCGGGACGTGCACAACCTCGACTACTACAAGAAGACGACCAACGGCCGGCTGCC |
| | CGTGAAGTGGATGGCGCCTGAGGCCTTGTTTGACCGAGTCTACACTCACCAGAGTGACGTCTGGTCCTTTGGGGTCCTGCTCTGGGA |
| | GATCTTCACGCTGGGGGGCTCCCCGTACCCCGGCATCCCTGTGGAGGAGCTCTTCAAGCTGCTGAAGGAGGGCCACCGCATGGACA |
| | AGCCCGCCAACTGCACACACGACCTGTACATGATCATGCGGGAGTGCTGGCATGCCGCGCCCTCCCAGAGGCCCACCTTCAAGCAG |
| | CTGGTGGAGGACCTGGACCGTGTCCTTACCGTGACGTCCACCGACGTAAAGGCGACACAGGAGGAGAACCGGGAGCTGAGGAGCA |
| | GGTGTGAGGAGCTCCACGGGAAGAACCTGGAACTGGGGAAGATCATGGACAGGTTCGAAGAGGTTGTGTACCAGGCCATGGAGGA |
| | AGTTCAGAAGCAGAAGGAACTTTCCAAAGCTGAAATCCAGAAAGTTCTAAAAGAAAAAGACCAACTTACCACAGATCTGAACTCC |
| | ATGGAGAAGTCCTTCTCCGACCTCTTCAAGCGTTTTGAGAAACAGAAAGAGGTGATCGAGGGCTACCGCAAGAACGAAGAGTCACT |
| | GAAGAAGTGCGTGGAGGATTACCTGGCAAGGATCACCCAGGAGGGCCAGAGGTACCAAGCCCTGAAGGCCCACGCGGAGGAGAA |
| | GCTGCAGCTGGCAAACGAGGAGATCGCCCAGGTCCGGAGCAAGGCCCAGGCGGAAGCGTTGGCCCTCCAGGCCAGCCTGAGGAAG |
| | GAGCAGATGCGCATCCAGTCGCTGGAGAAGACAGTGGAGCAGAAGACTAAAGAGAACGAGGAGCTGACCAGGATCTGCGACGAC |
| | CTCATCTCCAAGATGGAGAAGATCTGA |
| FGFR3:TACC3 v3 | >ATGGGCGCCCCTGCCTGCGCCCTCGCGCTCTGCGTGGCCGTGGCCATCGTGGCCGGCGCCTCCTCGGAGTCCTTGGGGACGGAGCA |
| (3376 base | GCGCGTCGTGGGGCGAGCGGCAGAAGTCCCGGGCCCAGAGCCCGGCCAGCAGGAGCAGTTGGTCTTCGGCAGCGGGGATGCTGTG |
| pairs) | GAGCTGAGCTGTCCCCCGCCCGGGGGTGGTCCCATGGGGCCCACTGTCTGGGTCAAGGATGGCACAGGGCTGGTGCCCTCGGAGCG |
| (SEQ ID NO: 57) | TGTCCTGGTGGGGCCCCAGCGGCTGCAGGTGCTGAATGCCTCCCACGAGGACTCCGGGGCCTACAGCTGCCGGCAGCGGCTCACGC |
| | AGCGCGTACTGTGCCACTTCAGTGTGCGGGTGACAGACGCTCCATCCTCGGGAGATGACGAAGACGGGGAGGACGAGGCTGAGGA |
| | CACAGGTGTGGACACAGGGGCCCCTTACTGGACACGGCCCGAGCGGATGGACAAGAAGCTGCTGGCCGTGCCGGCCGCCAACACC |
| | GTCCGCTTCCGCTGCCCAGCCGCTGGCAACCCCACTCCCTCCATCTCCTGGCTGAAGAACGGCAGGGAGTTCCGCGGCGAGCACCG |
| | CATTGGAGGCATCAAGCTGCGGCATCAGCAGTGGAGCCTGGTCATGGAAAGCGTGGTGCCCTCGGACCGCGGCAACTACACCTGCG |
| | TCGTGGAGAACAAGTTTGGCAGCATCCGGCAGACGTACACGCTGGACGTGCTGGAGCGCTCCCCGCACCGGCCCATCCTGCAGGCG |
| | GGGCTGCCGGCCAACCAGACGGCGGTGCTGGGCAGCGACGTGGAGTTCCACTGCAAGGTGTACAGTGACGCACAGCCCCACATCC |
| | AGTGGCTCAAGCACGTGGAGGTGAATGGCAGCAAGGTGGGCCCGGACGGCACACCCTACGTTACCGTGCTCAAGACGGCGGGCGC |
| | TAACACCACCGACAAGGAGCTAGAGGTTCTCTCCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGAGGAGCTGGTGGAGGCTGACGAGGCGGGCAGT |
| | GTGTATGCAGGCATCCTCAGCTACGGGGTGGGCTTCTTCCTGTTCATCCTGGTGGTGGCGGCTGTGACGCTCTGCCGCCTGCGCAGC |
| | CCCCCCAAGAAAGGCCTGGGCTCCCCCACCGTGCACAAGATCTCCCGCTTCCCGCTCAAGCGACAGGTGTCCCTGGAGTCCAACGC |
| | GTCCATGAGCTCCAACACACCACTGGTGCGCATCGCAAGGCTGTCCTCAGGGGAGGGCCCCACGCTGGCCAATGTCTCCGAGCTCG |
| | AGCTGCCTGCCGACCCCAAATGGGAGCTGTCTCGGGCCCGGCTGACCCTGGGCAAGCCCCTTGGGGAGGGCTGCTTCGGCCAGGTG |
| | GTCATGGCGGAGGCCATCGGCATTGACAAGGACCGGGCCGCCAAGCCTGTCACCGTAGCCGTGAAGATGCTGAAAGACGATGCCA |
| | CTGACAAGGACCTGTCGGACCTGGTGTCTGAGATGGAGATGATGAAGATGATCGGGAAACACAAAAACATCATCAACCTGCTGGG |
| | CGCCTGCACGCAGGGCGGGCCCCTGTACGTGCTGGTGGAGTACGCGGCCAAGGGTAACCTGCGGGAGTTTCTGCGGGCGCGGCGG |
| | CCCCCGGGCCTGGACTACTCCTTCGACACCTGCAAGCCGCCCGAGGAGCAGCTCACCTTCAAGGACCTGGTGTCCTGTGCCTACCA |
| | GGTGGCCCGGGGCATGGAGTACTTGGCCTCCCAGAAGTGCATCCACAGGGACCTGGCTGCCCGCAATGTGCTGGTGACCGAGGACA |
| | ACGTGATGAAGATCGCAGACTTCGGGCTGGCCCGGGACGTGCACAACCTCGACTACTACAAGAAGACGACCAACGGCCGGCTGCC |
| | CGTGAAGTGGATGGCGCCTGAGGCCTTGTTTGACCGAGTCTACACTCACCAGAGTGACGTCTGGTCCTTTGGGGTCCTGCTCTGGGA |
| | GATCTTCACGCTGGGGGGCTCCCCGTACCCCGGCATCCCTGTGGAGGAGCTCTTCAAGCTGCTGAAGGAGGGCCACCGCATGGACA |
| | AGCCCGCCAACTGCACACACGACCTGTACATGATCATGCGGGAGTGCTGGCATGCCGCGCCCTCCCAGAGGCCCACCTTCAAGCAG |
| | CTGGTGGAGGACCTGGACCGTGTCCTTACCGTGACGTCCACCGACGTGCCAGGCCCACCCCCAGGTGTTCCCGCGCCTGGGGGCCC |
| | ACCCCTGTCCACCGGACCTATAGTGGACCTGCTCCAGTACAGCCAGAAGGACCTGGATGCAGTGGTAAAGGCGACACAGGAGGAG |
| | AACCGGGAGCTGAGGAGCAGGTGTGAGGAGCTCCACGGGAAGAACCTGGAACTGGGGAAGATCATGGACAGGTTCGAAGAGGTT |
| | GTGTACCAGGCCATGGAGGAAGTTCAGAAGCAGAAGGAACTTTCCAAAGCTGAAATCCAGAAAGTTCTAAAAGAAAAAGACCAAC |
| | TTACCACAGATCTGAACTCCATGGAGAAGTCCTTCTCCGACCTCTTCAAGCGTTTTGAGAAACAGAAAGAGGTGATCGAGGGCTAC |
| | CGCAAGAACGAAGAGTCACTGAAGAAGTGCGTGGAGGATTACCTGGCAAGGATCACCCAGGAGGGCCAGAGGTACCAAGCCCTGA |
| | AGGCCCACGCGGAGGAGAAGCTGCAGCTGGCAAACGAGGAGATCGCCCAGGTCCGGAGCAAGGCCCAGGCGGAAGCGTTGGCCC |
| | TCCAGGCCAGCCTGAGGAAGGAGCAGATGCGCATCCAGTCGCTGGAGAAGACAGTGGAGCAGAAGACTAAAGAGAACGAGGAGC |
| | TGACCAGGATCTGCGACGACCTCATCTCCAAGATGGAGAAGATCTGA |
| FGFR3 | >ATGGGCGCCCCTGCCTGCGCCCTCGCGCTCTGCGTGGCCGTGGCCATCGTGGCCGGCGCCTCCTCGGAGTCCTTGGGGACGGAGCA |
| Intron:TACC3 | GCGCGTCGTGGGGCGAGCGGCAGAAGTCCCGGGCCCAGAGCCCGGCCAGCAGGAGCAGTTGGTCTTCGGCAGCGGGGATGCTGTG |
| (4463 base | GAGCTGAGCTGTCCCCCGCCCGGGGGTGGTCCCATGGGGCCCACTGTCTGGGTCAAGGATGGCACAGGGCTGGTGCCCTCGGAGCG |
| pairs) | TGTCCTGGTGGGGCCCCAGCGGCTGCAGGTGCTGAATGCCTCCCACGAGGACTCCGGGGCCTACAGCTGCCGGCAGCGGCTCACGC |
| (SEQ ID NO: 58) | AGCGCGTACTGTGCCACTTCAGTGTGCGGGTGACAGACGCTCCATCCTCGGGAGATGACGAAGACGGGGAGGACGAGGCTGAGGA |
| | CACAGGTGTGGACACAGGGGCCCCTTACTGGACACGGCCCGAGCGGATGGACAAGAAGCTGCTGGCCGTGCCGGCCGCCAACACC |
| | GTCCGCTTCCGCTGCCCAGCCGCTGGCAACCCCACTCCCTCCATCTCCTGGCTGAAGAACGGCAGGGAGTTCCGCGGCGAGCACCG |
| | CATTGGAGGCATCAAGCTGCGGCATCAGCAGTGGAGCCTGGTCATGGAAAGCGTGGTGCCCTCGGACCGCGGCAACTACACCTGCG |
| | TCGTGGAGAACAAGTTTGGCAGCATCCGGCAGACGTACACGCTGGACGTGCTGGAGCGCTCCCCGCACCGGCCCATCCTGCAGGCG |
| | GGGCTGCCGGCCAACCAGACGGCGGTGCTGGGCAGCGACGTGGAGTTCCACTGCAAGGTGTACAGTGACGCACAGCCCCACATCC |
| | AGTGGCTCAAGCACGTGGAGGTGAATGGCAGCAAGGTGGGCCCGGACGGCACACCCTACGTTACCGTGCTCAAGACGGCGGGCGC |
| | TAACACCACCGACAAGGAGCTAGAGGTTCTCTCCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGAGGAGCTGGTGGAGGCTGACGAGGCGGGCAGT |
| | GTGTATGCAGGCATCCTCAGCTACGGGGTGGGCTTCTTCCTGTTCATCCTGGTGGTGGCGGCTGTGACGCTCTGCCGCCTGCGCAGC |
| | CCCCCCAAGAAAGGCCTGGGCTCCCCCACCGTGCACAAGATCTCCCGCTTCCCGCTCAAGCGACAGGTGTCCCTGGAGTCCAACGC |
| | GTCCATGAGCTCCAACACACCACTGGTGCGCATCGCAAGGCTGTCCTCAGGGGAGGGCCCCACGCTGGCCAATGTCTCCGAGCTCG |
| | AGCTGCCTGCCGACCCCAAATGGGAGCTGTCTCGGGCCCGGCTGACCCTGGGCAAGCCCCTTGGGGAGGGCTGCTTCGGCCAGGTG |
| | GTCATGGCGGAGGCCATCGGCATTGACAAGGACCGGGCCGCCAAGCCTGTCACCGTAGCCGTGAAGATGCTGAAAGACGATGCCA |
| | CTGACAAGGACCTGTCGGACCTGGTGTCTGAGATGGAGATGATGAAGATGATCGGGAAACACAAAAACATCATCAACCTGCTGGG |
| | CGCCTGCACGCAGGGCGGGCCCCTGTACGTGCTGGTGGAGTACGCGGCCAAGGGTAACCTGCGGGAGTTTCTGCGGGCGCGGCGG |
| | CCCCCGGGCCTGGACTACTCCTTCGACACCTGCAAGCCGCCCGAGGAGCAGCTCACCTTCAAGGACCTGGTGTCCTGTGCCTACCA |
| | GGTGGCCCGGGGCATGGAGTACTTGGCCTCCCAGAAGTGCATCCACAGGGACCTGGCTGCCCGCAATGTGCTGGTGACCGAGGACA |
| | ACGTGATGAAGATCGCAGACTTCGGGCTGGCCCGGGACGTGCACAACCTCGACTACTACAAGAAGACGACCAACGGCCGGCTGCC |
| | CGTGAAGTGGATGGCGCCTGAGGCCTTGTTTGACCGAGTCTACACTCACCAGAGTGACGTCTGGTCCTTTGGGGTCCTGCTCTGGGA |
| | GATCTTCACGCTGGGGGGCTCCCCGTACCCCGGCATCCCTGTGGAGGAGCTCTTCAAGCTGCTGAAGGAGGGCCACCGCATGGACA |
| | AGCCCGCCAACTGCACACACGACCTGTACATGATCATGCGGGAGTGCTGGCATGCCGCGCCCTCCCAGAGGCCCACCTTCAAGCAG |
| | CTGGTGGAGGACCTGGACCGTGTCCTTACCGTGACGTCCACCGACgtgagtgctggctctggcctggtgccacccgcctatgcccct |
| | ccccctgccgtccccggccatcctgccccccagagtgctgaggtgtggggcgggccttTCTGGCCCAGGTGCCCTGGCTGACCTGGA |
| | CTGCTCAAGCTCTTCCCAGAGCCCAGGAAGTTCTGAGAACCAAATGGTGTCTCCAGGAAAAGTGTCTGGCAGCCCTGAGCAAGCCGT |
| | GGAGGAAAACCTTAGTTCCTATTCCTTAGACAGAAGAGTGACACCCGCCTCTGAGACCCTAGAAGACCCTTGCAGGACAGAGTCCC |
| | AGCACAAAGCGGAGACTCCGCACGGAGCCGAGGAAGAATGCAAAGCGGAGACTCCGCACGGAGCCGAGGAGGAATGCCGGCACGGT |
| | GGGGTCTGTGCTCCCGCAGCAGTGGCCACTTCGCCTCCTGGTGCAATCCCTAAGGAAGCCTGCGGAGGAGCACCCCTGCAGGGTCTG |
| | CCTGGCGAAGCCCTGGGCTGCCCTGCGGGTGTGGGCACCCCCGTGCCAGCAGATGGCACTCAGACCCTTACCTGTGCACACACCTCT |
| | GCTCCTGAGAGCACAGCCCCAACCAACCACCTGGTGGCTGGCAGGGCCATGACCCTGAGTCCTCAGGAAGAAGTGGCTGCAGGCCAA |
| | ATGGCCAGCTCCTCGAGGAGCGGACCTGTAAAACTAGAATTTGATGTATCTGATGGCGCCACCAGCAAAAGGGCACCCCCACCAAG |
| | GAGACTGGGAGAGAGGTCCGGCCTCAAGCCTCCCTTGAGGAAAGCAGCAGTGAGGCAGCAAAAGGCCCCGCAGGAGGTGGAGGAGGA |
| | CGACGGTAGGAGCGGAGCAGGAGAGGACCCCCCCATGCCAGCTTCTCGGGGCTCTTACCACCTCGACTGGGACAAAATGGATGACC |
| | CAAACTTCATCCCGTTCGGAGGTGACACCAAGTCTGGTTGCAGTGAGGCCCAGCCCCCAGAAAGCCCTGAGACCAGGCTGGGCCA |
| | GCCAGCGGCTGAACAGTTGCATGCTGGGCCTGCCACGGAGGAGCCAGGTCCCTGTCTGAGCCAGCAGCTGCATTCAGCCTCAGCGG |
| | AGGACACGCCTGTGGTGCAGTTGGCAGCCGAGACCCCAACAGCAGAGAGCAAGGAGAGAGCCTTGAACTCTGCCAGCACCTCGCTT |
| | CCCACAAGCTGTCCAGGCAGTGAGCCAGTGCCCACCCATCAGCAGGGGCAGCCTGCCTTGGAGCTGAAAGAGGAGAGCTTCAGAGA |
| | CCCCGCTGAGGTTCTAGGCACGGGCGCGGAGGTGGATTACCTGGAGCAGTTTGGAACTTCCTCGTTTAAGGAGTCGGCCTTGAGGAA |
| | GCAGTCCTTATACCTCAAGTTCGACCCCCTCCTGAGGGACAGTCCTGGTAGACCAGTGCCCGTGGCCACCGAGACCAGCAGCATGC |
| | ACGGTGCAAATGAGACTCCCTCAGGACGTCCGCGGGAAGCCAAGCTTGTGGAGTTCGATTTCTTGGGAGCACTGGACATTCCTGTG |
| | CCAGGCCCACCCCCAGGTGTTCCCGCGCCTGGGGGCCCACCCCTGTCCACCGGACCTATAGTGGACCTGCTCCAGTACAGCCAGAAG |
| | GACCTGGATGCAGTGGTAAAGGCGACACAGGAGGAGAACCGGGAGCTGAGGAGCAGGTGTGAGGAGCTCCACGGGAAGAACCTGGA |
| | ACTGGGGAAGATCATGGACAGGTTCGAAGAGGTTGTGTACCAGGCCATGGAGGAAGTTCAGAAGCAGAAGGAACTTTCCAAAGCTG |
| | AAATCCAGAAAGTTCTAAAAGAAAAAGACCAACTTACCACAGATCTGAACTCCATGGAGAAGTCCTTCTCCGACCTCTTCAAGCGTT |
| | TTGAGAAACAGAAAGAGGTGATCGAGGGCTACCGCAAGAACGAAGAGTCACTGAAGAAGTGCGTGGAGGATTACCTGGCAAGGATC |
| | ACCCAGGAGGGCCAGAGGTACCAAGCCCTGAAGGCCCACGCGGAGGAGAAGCTGCAGCTGGCAAACGAGGAGATCGCCCAGGTCCG |
| | GAGCAAGGCCCAGGCGGAAGCGTTGGCCCTCCAGGCCAGCCTGAGGAAGGAGCAGATGCGCATCCAGTCGCTGGAGAAGACAGTGG |
| | AGCAGAAGACTAAAGAGAACGAGGAGCTGACCAGGATCTGCGACGACCTCATCTCCAAGATGGAGAAGATCTGA |
| FGFR3:BAIAP2L1 | >ATGGGCGCCCCTGCCTGCGCCCTCGCGCTCTGCGTGGCCGTGGCCATCGTGGCCGGCGCCTCCTCGGAGTCCTTGGGGACGGAGCA |
| (3765 base | GCGCGTCGTGGGGCGAGCGGCAGAAGTCCCGGGCCCAGAGCCCGGCCAGCAGGAGCAGTTGGTCTTCGGCAGCGGGGATGCTGTG |
| pairs) | GAGCTGAGCTGTCCCCCGCCCGGGGGTGGTCCCATGGGGCCCACTGTCTGGGTCAAGGATGGCACAGGGCTGGTGCCCTCGGAGCG |
| (SEQ ID NO: 59) | TGTCCTGGTGGGGCCCCAGCGGCTGCAGGTGCTGAATGCCTCCCACGAGGACTCCGGGGCCTACAGCTGCCGGCAGCGGCTCACGC |
| | AGCGCGTACTGTGCCACTTCAGTGTGCGGGTGACAGACGCTCCATCCTCGGGAGATGACGAAGACGGGGAGGACGAGGCTGAGGA |
| | CACAGGTGTGGACACAGGGGCCCCTTACTGGACACGGCCCGAGCGGATGGACAAGAAGCTGCTGGCCGTGCCGGCCGCCAACACC |
| | GTCCGCTTCCGCTGCCCAGCCGCTGGCAACCCCACTCCCTCCATCTCCTGGCTGAAGAACGGCAGGGAGTTCCGCGGCGAGCACCG |
| | CATTGGAGGCATCAAGCTGCGGCATCAGCAGTGGAGCCTGGTCATGGAAAGCGTGGTGCCCTCGGACCGCGGCAACTACACCTGCG |
| | TCGTGGAGAACAAGTTTGGCAGCATCCGGCAGACGTACACGCTGGACGTGCTGGAGCGCTCCCCGCACCGGCCCATCCTGCAGGCG |
| | GGGCTGCCGGCCAACCAGACGGCGGTGCTGGGCAGCGACGTGGAGTTCCACTGCAAGGTGTACAGTGACGCACAGCCCCACATCC |
| | AGTGGCTCAAGCACGTGGAGGTGAATGGCAGCAAGGTGGGCCCGGACGGCACACCCTACGTTACCGTGCTCAAGTCCTGGATCAGT |
| | GAGAGTGTGGAGGCCGACGTGCGCCTCCGCCTGGCCAATGTGTCGGAGCGGGACGGGGGCGAGTACCTCTGTCGAGCCACCAATTT |
| | CATAGGCGTGGCCGAGAAGGCCTTTTGGCTGAGCGTTCACGGGCCCCGAGCAGCCGAGGAGGAGCTGGTGGAGGCTGACGAGGCG |
| | GGCAGTGTGTATGCAGGCATCCTCAGCTACGGGGTGGGCTTCTTCCTGTTCATCCTGGTGGTGGCGGCTGTGACGCTCTGCCGCCTG |
| | CGCAGCCCCCCCAAGAAAGGCCTGGGCTCCCCCACCGTGCACAAGATCTCCCGCTTCCCGCTCAAGCGACAGGTGTCCCTGGAGTC |
| | CAACGCGTCCATGAGCTCCAACACACCACTGGTGCGCATCGCAAGGCTGTCCTCAGGGGAGGGCCCCACGCTGGCCAATGTCTCCG |
| | AGCTCGAGCTGCCTGCCGACCCCAAATGGGAGCTGTCTCGGGCCCGGCTGACCCTGGGCAAGCCCCTTGGGGAGGGCTGCTTCGGC |
| | CAGGTGGTCATGGCGGAGGCCATCGGCATTGACAAGGACCGGGCCGCCAAGCCTGTCACCGTAGCCGTGAAGATGCTGAAAGACG |
| | ATGCCACTGACAAGGACCTGTCGGACCTGGTGTCTGAGATGGAGATGATGAAGATGATCGGGAAACACAAAAACATCATCAACCT |
| | GCTGGGCGCCTGCACGCAGGGCGGGCCCCTGTACGTGCTGGTGGAGTACGCGGCCAAGGGTAACCTGCGGGAGTTTCTGCGGGCGC |
| | GGCGGCCCCCGGGCCTGGACTACTCCTTCGACACCTGCAAGCCGCCCGAGGAGCAGCTCACCTTCAAGGACCTGGTGTCCTGTGCC |
| | TACCAGGTGGCCCGGGGCATGGAGTACTTGGCCTCCCAGAAGTGCATCCACAGGGACCTGGCTGCCCGCAATGTGCTGGTGACCGA |
| | GGACAACGTGATGAAGATCGCAGACTTCGGGCTGGCCCGGGACGTGCACAACCTCGACTACTACAAGAAGACGACCAACGGCCGG |
| | CTGCCCGTGAAGTGGATGGCGCCTGAGGCCTTGTTTGACCGAGTCTACACTCACCAGAGTGACGTCTGGTCCTTTGGGGTCCTGCTC |
| | TGGGAGATCTTCACGCTGGGGGGCTCCCCGTACCCCGGCATCCCTGTGGAGGAGCTCTTCAAGCTGCTGAAGGAGGGCCACCGCAT |
| | GGACAAGCCCGCCAACTGCACACACGACCTGTACATGATCATGCGGGAGTGCTGGCATGCCGCGCCCTCCCAGAGGCCCACCTTCA |
| | AGCAGCTGGTGGAGGACCTGGACCGTGTCCTTACCGTGACGTCCACCGACAATGTTATGGAACAGTTCAATCCTGGGCTGCGAAAT |
| | TTAATAAACCTGGGGAAAAATTATGAGAAAGCTGTAAACGCTATGATCCTGGCAGGAAAAGCCTACTACGATGGAGTGGCCAAGA |
| | TCGGTGAGATTGCCACTGGGTCCCCCGTGTCAACTGAACTGGGACATGTCCTCATAGAGATTTCAAGTACCCACAAGAAACTCAAC |
| | GAGAGTCTTGATGAAAATTTTAAAAAATTCCACAAAGAGATTATCCATGAGCTGGAGAAGAAGATAGAACTTGACGTGAAATATAT |
| | GAACGCAACTCTAAAAAGATACCAAACAGAACACAAGAATAAATTAGAGTCTTTGGAGAAATCCCAAGCTGAGTTGAAGAAGATC |
| | AGAAGGAAAAGCCAAGGAAGCCGAAACGCACTCAAATATGAACACAAAGAAATTGAGTATGTGGAGACCGTTACTTCTCGTCAGA |
| | GTGAAATCCAGAAATTCATTGCAGATGGTTGCAAAGAGGCTCTGCTTGAAGAGAAGAGGCGCTTCTGCTTTCTGGTTGATAAGCAC |
| | TGTGGCTTTGCAAACCACATACATTATTATCACTTACAGTCTGCAGAACTACTGAATTCCAAGCTGCCTCGGTGGCAGGAGACCTGT |
| | GTTGATGCCATCAAAGTGCCAGAGAAAATCATGAATATGATCGAAGAAATAAAGACCCCAGCCTCTACCCCCGTGTCTGGAACTCC |
| | TCAGGCTTCACCCATGATCGAGAGAAGCAATGTGGTTAGGAAAGATTACGACACCCTTTCTAAATGCTCACCAAAGATGCCCCCCG |
| | CTCCTTCAGGCAGAGCATATACCAGTCCCTTGATCGATATGTTTAATAACCCAGCCACGGCTGCCCCGAATTCACAAAGGGTAAAT |
| | AATTCAACAGGTACTTCCGAAGATCCCAGTTTACAGCGATCAGTTTCGGTTGCAACGGGACTGAACATGATGAAGAAGCAGAAAGT |
| | GAAGACCATCTTCCCGCACACTGCGGGCTCCAACAAGACCTTACTCAGCTTTGCACAGGGAGATGTCATCACGCTGCTCATCCCCG |
| | AGGAGAAGGATGGCTGGCTCTATGGAGAACACGACGTGTCCAAGGCGAGGGGTTGGTTCCCGTCGTCGTACACGAAGTTGCTGGA |
| | AGAAAATGAGACAGAAGCAGTGACCGTGCCCACGCCAAGCCCCACACCAGTGAGAAGCATCAGCACCGTGAACTTGTCTGAGAAT |
| | AGCAGTGTTGTCATCCCCCCACCCGACTACTTGGAATGCTTGTCCATGGGGGCAGCTGCCGACAGGAGAGCAGATTCGGCCAGGAC |
| | GACATCCACCTTTAAGGCCCCAGCGTCCAAGCCCGAGACCGCGGCTCCTAACGATGCCAACGGGACTGCAAAGCCGCCTTTTCTCA |
| | GCGGAGAAAACCCCTTTGCCACTGTGAAACTCCGCCCGACTGTGACGAATGATCGCTCGGCACCCATCATTCGATGA |
| FGFR2:BICC1 | >ATGGTCAGCTGGGGTCGTTTCATCTGCCTGGTCGTGGTCACCATGGCAACCTTGTCCCTGGCCCGGCCCTCCTTCAGTTTAGTTGAG |
| (5830 base | GATACCACATTAGAGCCAGAAGAGCCACCAACCAAATACCAAATCTCTCAACCAGAAGTGTACGTGGCTGCGCCAGGGGAGTCGC |
| pairs) | TAGAGGTGCGCTGCCTGTTGAAAGATGCCGCCGTGATCAGTTGGACTAAGGATGGGGTGCACTTGGGGCCCAACAATAGGACAGTG |
| (SEQ ID NO: 60) | CTTATTGGGGAGTACTTGCAGATAAAGGGCGCCACGCCTAGAGACTCCGGCCTCTATGCTTGTACTGCCAGTAGGACTGTAGACAG |
| | TGAAACTTGGTACTTCATGGTGAATGTCACAGATGCCATCTCATCCGGAGATGATGAGGATGACACCGATGGTGCGGAAGATTTTG |
| | TCAGTGAGAACAGTAACAACAAGAGAGCACCATACTGGACCAACACAGAAAAGATGGAAAAGCGGCTCCATGCTGTGCCTGCGGC |
| | CAACACTGTCAAGTTTCGCTGCCCAGCCGGGGGGAACCCAATGCCAACCATGCGGTGGCTGAAAAACGGGAAGGAGTTTAAGCAG |
| | GAGCATCGCATTGGAGGCTACAAGGTACGAAACCAGCACTGGAGCCTCATTATGGAAAGTGTGGTCCCATCTGACAAGGGAAATT |
| | ATACCTGTGTAGTGGAGAATGAATACGGGTCCATCAATCACACGTACCACCTGGATGTTGTGGAGCGATCGCCTCACCGGCCCATC |
| | CTCCAAGCCGGACTGCCGGCAAATGCCTCCACAGTGGTCGGAGGAGACGTAGAGTTTGTCTGCAAGGTTTACAGTGATGCCCAGCC |
| | CCACATCCAGTGGATCAAGCACGTGGAAAAGAACGGCAGTAAATACGGGCCCGACGGGCTGCCCTACCTCAAGGTTCTCAAGGCC |
| | GCCGGTGTTAACACCACGGACAAAGAGATTGAGGTTCTCTATATTCGGAATGTAACTTTTGAGGACGCTGGGGAATATACGTGCTT |
| | GGCGGGTAATTCTATTGGGATATCCTTTCACTCTGCATGGTTGACAGTTCTGCCAGCGCCTGGAAGAGAAAAGGAGATTACAGCTTC |
| | CCCAGACTACCTGGAGATAGCCATTTACTGCATAGGGGTCTTCTTAATCGCCTGTATGGTGGTAACAGTCATCCTGTGCCGAATGAA |
| | GAACACGACCAAGAAGCCAGACTTCAGCAGCCAGCCGGCTGTGCACAAGCTGACCAAACGTATCCCCCTGCGGAGACAGGTAACA |
| | GTTTCGGCTGAGTCCAGCTCCTCCATGAACTCCAACACCCCGCTGGTGAGGATAACAACACGCCTCTCTTCAACGGCAGACACCCCC |
| | ATGCTGGCAGGGGTCTCCGAGTATGAACTTCCAGAGGACCCAAAATGGGAGTTTCCAAGAGATAAGCTGACACTGGGCAAGCCCCT |
| | GGGAGAAGGTTGCTTTGGGCAAGTGGTCATGGCGGAAGCAGTGGGAATTGACAAAGACAAGCCCAAGGAGGCGGTCACCGTGGCC |
| | GTGAAGATGTTGAAAGATGATGCCACAGAGAAAGACCTTTCTGATCTGGTGTCAGAGATGGAGATGATGAAGATGATTGGGAAAC |
| | ACAAGAATATCATAAATCTTCTTGGAGCCTGCACACAGGATGGGCCTCTCTATGTCATAGTTGAGTATGCCTCTAAAGGCAACCTCC |
| | GAGAATACCTCCGAGCCCGGAGGCCACCCGGGATGGAGTACTCCTATGACATTAACCGTGTTCCTGAGGAGCAGATGACCTTCAAG |
| | GACTTGGTGTCATGCACCTACCAGCTGGCCAGAGGCATGGAGTACTTGGCTTCCCAAAAATGTATTCATCGAGATTTAGCAGCCAG |
| | AAATGTTTTGGTAACAGAAAACAATGTGATGAAAATAGCAGACTTTGGACTCGCCAGAGATATCAACAATATAGACTATTACAAAA |
| | AGACCACCAATGGGCGGCTTCCAGTCAAGTGGATGGCTCCAGAAGCCCTGTTTGATAGAGTATACACTCATCAGAGTGATGTCTGG |
| | TCCTTCGGGGTGTTAATGTGGGAGATCTTCACTTTAGGGGGCTCGCCCTACCCAGGGATTCCCGTGGAGGAACTTTTTAAGCTGCTG |
| | AAGGAAGGACACAGAATGGATAAGCCAGCCAACTGCACCAACGAACTGTACATGATGATGAGGGACTGTTGGCATGCAGTGCCCT |
| | CCCAGAGACCAACGTTCAAGCAGTTGGTAGAAGACTTGGATCGAATTCTCACTCTCACAACCAATGAGATCATGGAGGAAACAAAT |
| | ACGCAGATTGCTTGGCCATCAAAACTGAAGATCGGAGCCAAATCCAAGAAAGATCCCCATATTAAGGTTTCTGGAAAGAAAGAAG |
| | ATGTTAAAGAAGCCAAGGAAATGATCATGTCTGTCTTAGACACAAAAAGCAATCGAGTCACACTGAAGATGGATGTTTCACATACA |
| | GAACATTCACATGTAATCGGCAAAGGTGGCAACAATATTAAAAAAGTGATGGAAGAAACCGGATGCCATATCCACTTTCCAGATTC |
| | CAACAGGAATAACCAAGCAGAAAAAAGCAACCAGGTATCTATAGCGGGACAACCAGCAGGAGTAGAATCTGCCCGAGTTAGAATT |
| | CGGGAGCTGCTTCCTTTGGTGCTGATGTTTGAGCTACCAATTGCTGGAATTCTTCAACCGGTTCCTGATCCTAATTCCCCCTCTATTC |
| | AGCATATATCACAAACGTACAATATTTCAGTATCATTTAAACAGCGTTCCCGAATGTATGGTGCTACTGTCATAGTACGAGGGTCTC |
| | AGAATAACACTAGTGCTGTGAAGGAAGGAACTGCCATGCTGTTAGAACATCTTGCTGGGAGCTTAGCATCAGCTATTCCTGTGAGC |
| | ACACAACTAGATATTGCAGCTCAACATCATCTCTTTATGATGGGTCGAAATGGGAGCAACATCAAACATATCATGCAGAGAACAGG |
| | TGCTCAGATCCACTTTCCTGATCCCAGTAATCCACAAAAGAAATCTACCGTCTACCTCCAGGGCACCATTGAGTCTGTCTGTCTTGC |
| | AAGGCAATATCTCATGGGTTGTCTTCCTCTTGTGTTGATGTTTGATATGAAGGAAGAAATTGAAGTAGATCCACAATTCATTGCGCA |
| | GTTGATGGAACAGCTTGATGTCTTCATCAGTATTAAACCAAAGCCCAAACAGCCAAGCAAGTCTGTGATTGTGAAAAGTGTTGAGC |
| | GAAATGCCTTAAATATGTATGAAGCAAGGAAATGTCTCCTCGGACTTGAAAGCAGTGGGGTTACCATAGCAACCAGTCCATCCCCA |
| | GCATCCTGCCCTGCCGGCCTGGCATGTCCCAGCCTGGATATCTTAGCTTCAGCAGGCCTTGGACTCACTGGACTAGGTCTTTTGGGA |
| | CCCACCACCTTATCTCTGAACACTTCAACAACCCCAAACTCACTCTTGAATGCTCTTAATAGCTCAGTCAGTCCTTTGCAAAGTCCA |
| | AGTTCTGGTACACCCAGCCCCACATTATGGGCACCCCCACTTGCTAATACTTCAAGTGCCACAGGTTTTTCTGCTATACCACACCTT |
| | ATGATTCCATCTACTGCCCAAGCCACATTAACTAATATTTTGTTGTCTGGAGTGCCCACCTATGGGCACACAGCTCCATCTCCCCCTC |
| | CTGGCTTGACTCCTGTTGATGTCCATATCAACAGTATGCAGACCGAAGGCAAAAAAATCTCTGCTGCTTTAAATGGACATGCACAGT |
| | CTCCAGATATAAAATATGGTGCAATATCCACTTCATCACTTGGAGAAAAAGTGCTGAGTGCAAATCACGGGGATCCGTCCATCCAG |
| | ACAAGTGGGTCTGAGCAGACATCTCCCAAATCAAGCCCCACTGAAGGTTGTAATGATGCTTTTGTTGAAGTAGGCATGCCTCGAAG |
| | TCCTTCCCATTCTGGGAATGCTGGTGACTTGAAACAGATGATGTGTCCCTCCAAGGTTTCCTGTGCCAAAAGGCAGACAGTGGAACT |
| | ATTGCAAGGCACGAAAAACTCACACTTACACAGCACTGACAGGTTGCTCTCAGACCCTGAACTGAGTGCTACCGAAAGCCCTTTGG |
| | CTGACAAGAAGGCTCCAGGGAGTGAGCGCGCTGCAGAGAGGGCAGCAGCTGCCCAGCAAAACTCCGAAAGGGCCCACCTTGCTCC |
| | ACGGTCATCATATGTCAACATGCAGGCATTTGACTATGAACAGAAGAAGCTATTAGCCACCAAAGCTATGTTAAAGAAACCAGTGG |
| | TGACGGAGGTCAGAACGCCCACAAATACCTGGAGTGGCCTGGGTTTTTCTAAATCCATGCCAGCTGAAACTATCAAGGAGTTGAGA |
| | AGGGCCAATCATGTGTCCTATAAGCCCACAATGACAACCACTTATGAGGGCTCATCCATGTCCCTTTCACGGTCCAACAGTCGTGAG |
| | CACTTGGGAGGTGGAAGCGAATCTGATAACTGGAGAGACCGAAATGGAATTGGACCTGGAAGTCATAGTGAATTTGCAGCTTCTAT |
| | TGGCAGCCCTAAGCGTAAACAAAACAAATCAACGGAACACTATCTCAGCAGTAGCAATTACATGGACTGCATTTCCTCGCTGACAG |
| | GAAGCAATGGCTGTAACTTAAATAGCTCTTTCAAAGGTTCTGACCTCCCTGAGCTCTTCAGCAAACTGGGCCTGGGCAAATACACA |
| | GATGTTTTCCAGCAACAAGAGATCGATCTTCAGACATTCCTCACTCTCACAGATCAGGATCTGAAGGAGCTGGGAATAACTACTTTT |
| | GGTGCCAGGAGGAAAATGCTGCTTGCAATTTCAGAACTAAATAAAAACCGAAGAAAGCTTTTTGAATCGCCAAATGCACGCACCTC |
| | TTTCCTGGAAGGTGGAGCGAGTGGAAGGCTACCCCGTCAGTATCACTCAGACATTGCTAGTGTCAGTGGCCGCTGGTAG |
| FGFR2:AFF3 | >ATGGTCAGCTGGGGTCGTTTCATCTGCCTGGTCGTGGTCACCATGGCAACCTTGTCCCTGGCCCGGCCCTCCTTCAGTTTAGTTGAG |
| (5109 base | GATACCACATTAGAGCCAGAAGAGCCACCAACCAAATACCAAATCTCTCAACCAGAAGTGTACGTGGCTGCGCCAGGGGAGTCGC |
| pairs) | TAGAGGTGCGCTGCCTGTTGAAAGATGCCGCCGTGATCAGTTGGACTAAGGATGGGGTGCACTTGGGGCCCAACAATAGGACAGTG |
| (SEQ ID NO: 61) | CTTATTGGGGAGTACTTGCAGATAAAGGGCGCCACGCCTAGAGACTCCGGCCTCTATGCTTGTACTGCCAGTAGGACTGTAGACAG |
| | TGAAACTTGGTACTTCATGGTGAATGTCACAGATGCCATCTCATCCGGAGATGATGAGGATGACACCGATGGTGCGGAAGATTTTG |
| | TCAGTGAGAACAGTAACAACAAGAGAGCACCATACTGGACCAACACAGAAAAGATGGAAAAGCGGCTCCATGCTGTGCCTGCGGC |
| | CAACACTGTCAAGTTTCGCTGCCCAGCCGGGGGGAACCCAATGCCAACCATGCGGTGGCTGAAAAACGGGAAGGAGTTTAAGCAG |
| | GAGCATCGCATTGGAGGCTACAAGGTACGAAACCAGCACTGGAGCCTCATTATGGAAAGTGTGGTCCCATCTGACAAGGGAAATT |
| | ATACCTGTGTAGTGGAGAATGAATACGGGTCCATCAATCACACGTACCACCTGGATGTTGTGGAGCGATCGCCTCACCGGCCCATC |
| | CTCCAAGCCGGACTGCCGGCAAATGCCTCCACAGTGGTCGGAGGAGACGTAGAGTTTGTCTGCAAGGTTTACAGTGATGCCCAGCC |
| | CCACATCCAGTGGATCAAGCACGTGGAAAAGAACGGCAGTAAATACGGGCCCGACGGGCTGCCCTACCTCAAGGTTCTCAAGGCC |
| | GCCGGTGTTAACACCACGGACAAAGAGATTGAGGTTCTCTATATTCGGAATGTAACTTTTGAGGACGCTGGGGAATATACGTGCTT |
| | GGCGGGTAATTCTATTGGGATATCCTTTCACTCTGCATGGTTGACAGTTCTGCCAGCGCCTGGAAGAGAAAAGGAGATTACAGCTTC |
| | CCCAGACTACCTGGAGATAGCCATTTACTGCATAGGGGTCTTCTTAATCGCCTGTATGGTGGTAACAGTCATCCTGTGCCGAATGAA |
| | GAACACGACCAAGAAGCCAGACTTCAGCAGCCAGCCGGCTGTGCACAAGCTGACCAAACGTATCCCCCTGCGGAGACAGGTAACA |
| | GTTTCGGCTGAGTCCAGCTCCTCCATGAACTCCAACACCCCGCTGGTGAGGATAACAACACGCCTCTCTTCAACGGCAGACACCCCC |
| | ATGCTGGCAGGGGTCTCCGAGTATGAACTTCCAGAGGACCCAAAATGGGAGTTTCCAAGAGATAAGCTGACACTGGGCAAGCCCCT |
| | GGGAGAAGGTTGCTTTGGGCAAGTGGTCATGGCGGAAGCAGTGGGAATTGACAAAGACAAGCCCAAGGAGGCGGTCACCGTGGCC |
| | GTGAAGATGTTGAAAGATGATGCCACAGAGAAAGACCTTTCTGATCTGGTGTCAGAGATGGAGATGATGAAGATGATTGGGAAAC |
| | ACAAGAATATCATAAATCTTCTTGGAGCCTGCACACAGGATGGGCCTCTCTATGTCATAGTTGAGTATGCCTCTAAAGGCAACCTCC |
| | GAGAATACCTCCGAGCCCGGAGGCCACCCGGGATGGAGTACTCCTATGACATTAACCGTGTTCCTGAGGAGCAGATGACCTTCAAG |
| | GACTTGGTGTCATGCACCTACCAGCTGGCCAGAGGCATGGAGTACTTGGCTTCCCAAAAATGTATTCATCGAGATTTAGCAGCCAG |
| | AAATGTTTTGGTAACAGAAAACAATGTGATGAAAATAGCAGACTTTGGACTCGCCAGAGATATCAACAATATAGACTATTACAAAA |
| | AGACCACCAATGGGCGGCTTCCAGTCAAGTGGATGGCTCCAGAAGCCCTGTTTGATAGAGTATACACTCATCAGAGTGATGTCTGG |
| | TCCTTCGGGGTGTTAATGTGGGAGATCTTCACTTTAGGGGGCTCGCCCTACCCAGGGATTCCCGTGGAGGAACTTTTTAAGCTGCTG |
| | AAGGAAGGACACAGAATGGATAAGCCAGCCAACTGCACCAACGAACTGTACATGATGATGAGGGACTGTTGGCATGCAGTGCCCT |
| | CCCAGAGACCAACGTTCAAGCAGTTGGTAGAAGACTTGGATCGAATTCTCACTCTCACAACCAATGAGGAGAGTAGATCTGGAGAA |
| | ACCAACAGCTGTGTTGAAGAAATAATCCGGGAGATGACCTGGCTTCCACCACTTTCTGCTATTCAAGCACCTGGCAAAGTGGAACC |
| | AACCAAATTTCCATTTCCAAATAAGGACTCTCAGCTTGTATCCTCTGGACACAATAATCCAAAGAAAGGTGATGCAGAGCCAGAGA |
| | GTCCAGACAGTGGCACATCGAATACATCAATGCTGGAAGATGACCTTAAGCTAAGCAGTGATGAAGAGGAGAATGAACAGCAGGC |
| | AGCTCAGAGAACGGCTCTCCGCGCTCTCTCTGACAGCGCCGTGGTCCAGCAGCCCAACTGCAGAACCTCGGTGCCTTCCAGCAAGG |
| | GCAGCAGCAGCAGCAGCAGCAGCGGCAGCAGCAGCTCCTCCAGCGACTCAGAGAGCAGCTCCGGATCTGACTCGGAGACCGAGAG |
| | CAGCTCCAGCGAGAGTGAGGGCAGCAAGCCCCCCCACTTCTCCAGCCCCGAGGCTGAACCGGCATCCTCTAACAAGTGGCAGCTGG |
| | ATAAATGGCTAAACAAAGTTAATCCCCACAAGCCTCCTATTCTGATCCAAAATGAAAGCCACGGGTCAGAGAGCAATCAGTACTAC |
| | AACCCGGTGAAAGAGGACGTCCAGGACTGTGGGAAAGTCCCCGACGTTTGCCAGCCCAGCCTGAGAGAGAAGGAGATCAAGAGCA |
| | CTTGCAAGGAGGAGCAAAGGCCAAGGACAGCCAACAAGGCCCCTGGGAGTAAAGGCGTGAAGCAGAAGTCCCCGCCCGCGGCCG |
| | TGGCCGTGGCGGTGAGCGCAGCCGCCCCGCCACCCGCAGTGCCCTGTGCGCCCGCGGAGAACGCGCCCGCGCCTGCCCGGAGGTCC |
| | GCGGGCAAGAAGCCCACCAGGCGCACCGAGAGGACCTCAGCCGGGGACGGCGCCAACTGCCACCGGCCCGAGGAGCCCGCGGCC |
| | GCGGACGCGCTGGGGACGAGCGTGGTGGTCCCCCCGGAGCCCACCAAAACCAGGCCCTGTGGCAACAACAGAGCGAGCCACCGCA |
| | AGGAGCTGCGCTCCTCCGTGACCTGCGAGAAGCGCCGCACGCGGGGGCTAAGCAGGATCGTCCCCAAATCCAAGGAGTTCATTGA |
| | GACAGAGTCGTCATCTTCATCCTCCTCCTCGGACTCCGACCTGGAGTCCGAGCAGGAGGAGTACCCTCTGTCCAAAGCACAGACCG |
| | TGGCTGCCTCTGCCTCCTCCGGGAATGATCAGAGGCTGAAGGAGGCCGCTGCCAACGGGGGCAGTGGTCCTAGGGCCCCTGTAGGC |
| | TCCATCAACGCCAGGACCACCAGTGACATCGCCAAGGAGCTGGAGGAGCAGTTCTACACACTGGTCCCCTTTGGCCGGAACGAACT |
| | TCTCTCCCCTCTAAAGGACAGTGATGAGATCAGGTCTCTCTGGGTCAAAATCGACCTGACCCTCCTGTCCAGGATCCCAGAACACCT |
| | GCCCCAGGAGCCAGGGGTATTGAGCGCCCCTGCCACCAAGGACTCTGAGAGCGCACCGCCCAGCCACACCTCGGACACACCTGCA |
| | GAAAAGGCTTTGCCAAAATCCAAGAGGAAACGCAAGTGTGACAACGAAGACGACTACAGGGAGATCAAGAAGTCCCAGGGAGAG |
| | AAAGACAGCTCTTCAAGACTGGCCACCTCCACCAGTAATACTTTGTCTGCAAACCACTGCAACATGAACATCAACAGTGTGGCAAT |
| | ACCAATAAATAAAAATGAAAAAATGCTTCGGTCGCCCATCTCACCCCTCTCTGATGCATCTAAACACAAATACACCAGCGAGGACT |
| | TAACTTCTTCCAGCCGACCTAATGGCAACAGTTTGTTTACTTCAGCCTCTTCCAGCAAAAAGCCTAAGGCCGACAGCCAGCTGCAGC |
| | CTCACGGCGGAGACCTCACGAAAGCAGCTCACAACAATTCTGAAAACATTCCCCTCCACAAGTCACGGCCGCAGACGAAGCCGTG |
| | GTCTCCAGGCTCCAACGGCCACAGGGACTGCAAGAGGCAGAAACTTGTCTTCGATGATATGCCTCGCAGTGCCGATTATTTTATGC |
| | AAGAAGCTAAACGAATGAAGCATAAAGCAGATGCAATGGTGGAAAAGTTTGGAAAGGCTTTGAACTATGCTGAAGCAGCATTGTC |
| | GTTTATCGAGTGTGGAAATGCAATGGAACAAGGCCCCATGGAATCCAAATCTCCTTATACGATGTATTCAGAAACAGTAGAGCTCA |
| | TCAGGTATGCTATGAGACTAAAAACCCACTCAGGCCCCAATGCCACACCAGAAGACAAACAACTGGCTGCATTATGTTACCGATGC |
| | CTGGCCCTCCTGTACTGGCGGATGTTTCGACTCAAAAGGGACCACGCTGTAAAGTATTCAAAAGCACTAATCGACTATTTCAAGAA |
| | CTCATCTAAAGCCGCCCAAGCCCCATCTCCGTGGGGGGCCAGTGGAAAGAGCACTGGAACCCCATCCCCCATGTCTCCCAACCCCT |
| | CTCCCGCCAGCTCCGTGGGGTCTCAGGGCAGCCTCTCCAACGCCAGCGCCCTGTCCCCGTCGACCATCGTCAGCATCCCACAGCGCA |
| | TCCACCAGATGGCGGCCAACCACGTCAGCATCACCAACAGCATCCTGCACAGCTACGACTACTGGGAGATGGCCGACAACCTGGCC |
| | AAGGAAAACCGAGAATTCTTCAACGACCTGGATCTGCTCATGGGGCCGGTCACCCTGCACAGCAGCATGGAGCACCTGGTCCAGTA |
| | CTCCCAACAGGGCCTGCACTGGCTGCGGAACAGCGCCCACCTGTCATAG |
| FGFR2:CASP7 | >ATGGTCAGCTGGGGTCGTTTCATCTGCCTGGTCGTGGTCACCATGGCAACCTTGTCCCTGGCCCGGCCCTCCTTCAGTTTAGTTGAG |
| (3213 base | GATACCACATTAGAGCCAGAAGAGCCACCAACCAAATACCAAATCTCTCAACCAGAAGTGTACGTGGCTGCGCCAGGGGAGTCGC |
| pairs) | TAGAGGTGCGCTGCCTGTTGAAAGATGCCGCCGTGATCAGTTGGACTAAGGATGGGGTGCACTTGGGGCCCAACAATAGGACAGTG |
| (SEQ ID NO: 62) | CTTATTGGGGAGTACTTGCAGATAAAGGGCGCCACGCCTAGAGACTCCGGCCTCTATGCTTGTACTGCCAGTAGGACTGTAGACAG |
| | TGAAACTTGGTACTTCATGGTGAATGTCACAGATGCCATCTCATCCGGAGATGATGAGGATGACACCGATGGTGCGGAAGATTTTG |
| | TCAGTGAGAACAGTAACAACAAGAGAGCACCATACTGGACCAACACAGAAAAGATGGAAAAGCGGCTCCATGCTGTGCCTGCGGC |
| | CAACACTGTCAAGTTTCGCTGCCCAGCCGGGGGGAACCCAATGCCAACCATGCGGTGGCTGAAAAACGGGAAGGAGTTTAAGCAG |
| | GAGCATCGCATTGGAGGCTACAAGGTACGAAACCAGCACTGGAGCCTCATTATGGAAAGTGTGGTCCCATCTGACAAGGGAAATT |
| | ATACCTGTGTAGTGGAGAATGAATACGGGTCCATCAATCACACGTACCACCTGGATGTTGTGGAGCGATCGCCTCACCGGCCCATC |
| | CTCCAAGCCGGACTGCCGGCAAATGCCTCCACAGTGGTCGGAGGAGACGTAGAGTTTGTCTGCAAGGTTTACAGTGATGCCCAGCC |
| | CCACATCCAGTGGATCAAGCACGTGGAAAAGAACGGCAGTAAATACGGGCCCGACGGGCTGCCCTACCTCAAGGTTCTCAAGGCC |
| | GCCGGTGTTAACACCACGGACAAAGAGATTGAGGTTCTCTATATTCGGAATGTAACTTTTGAGGACGCTGGGGAATATACGTGCTT |
| | GGCGGGTAATTCTATTGGGATATCCTTTCACTCTGCATGGTTGACAGTTCTGCCAGCGCCTGGAAGAGAAAAGGAGATTACAGCTTC |
| | CCCAGACTACCTGGAGATAGCCATTTACTGCATAGGGGTCTTCTTAATCGCCTGTATGGTGGTAACAGTCATCCTGTGCCGAATGAA |
| | GAACACGACCAAGAAGCCAGACTTCAGCAGCCAGCCGGCTGTGCACAAGCTGACCAAACGTATCCCCCTGCGGAGACAGGTAACA |
| | GTTTCGGCTGAGTCCAGCTCCTCCATGAACTCCAACACCCCGCTGGTGAGGATAACAACACGCCTCTCTTCAACGGCAGACACCCCC |
| | ATGCTGGCAGGGGTCTCCGAGTATGAACTTCCAGAGGACCCAAAATGGGAGTTTCCAAGAGATAAGCTGACACTGGGCAAGCCCCT |
| | GGGAGAAGGTTGCTTTGGGCAAGTGGTCATGGCGGAAGCAGTGGGAATTGACAAAGACAAGCCCAAGGAGGCGGTCACCGTGGCC |
| | GTGAAGATGTTGAAAGATGATGCCACAGAGAAAGACCTTTCTGATCTGGTGTCAGAGATGGAGATGATGAAGATGATTGGGAAAC |
| | ACAAGAATATCATAAATCTTCTTGGAGCCTGCACACAGGATGGGCCTCTCTATGTCATAGTTGAGTATGCCTCTAAAGGCAACCTCC |
| | GAGAATACCTCCGAGCCCGGAGGCCACCCGGGATGGAGTACTCCTATGACATTAACCGTGTTCCTGAGGAGCAGATGACCTTCAAG |
| | GACTTGGTGTCATGCACCTACCAGCTGGCCAGAGGCATGGAGTACTTGGCTTCCCAAAAATGTATTCATCGAGATTTAGCAGCCAG |
| | AAATGTTTTGGTAACAGAAAACAATGTGATGAAAATAGCAGACTTTGGACTCGCCAGAGATATCAACAATATAGACTATTACAAAA |
| | AGACCACCAATGGGCGGCTTCCAGTCAAGTGGATGGCTCCAGAAGCCCTGTTTGATAGAGTATACACTCATCAGAGTGATGTCTGG |
| | TCCTTCGGGGTGTTAATGTGGGAGATCTTCACTTTAGGGGGCTCGCCCTACCCAGGGATTCCCGTGGAGGAACTTTTTAAGCTGCTG |
| | AAGGAAGGACACAGAATGGATAAGCCAGCCAACTGCACCAACGAACTGTACATGATGATGAGGGACTGTTGGCATGCAGTGCCCT |
| | CCCAGAGACCAACGTTCAAGCAGTTGGTAGAAGACTTGGATCGAATTCTCACTCTCACAACCAATGAGATGGCAGATGATCAGGGC |
| | TGTATTGAAGAGCAGGGGGTTGAGGATTCAGCAAATGAAGATTCAGTGGATGCTAAGCCAGACCGGTCCTCGTTTGTACCGTCCCT |
| | CTTCAGTAAGAAGAAGAAAAATGTCACCATGCGATCCATCAAGACCACCCGGGACCGAGTGCCTACATATCAGTACAACATGAATT |
| | TTGAAAAGCTGGGCAAATGCATCATAATAAACAACAAGAACTTTGATAAAGTGACAGGTATGGGCGTTCGAAACGGAACAGACAA |
| | AGATGCCGAGGCGCTCTTCAAGTGCTTCCGAAGCCTGGGTTTTGACGTGATTGTCTATAATGACTGCTCTTGTGCCAAGATGCAAGA |
| | TCTGCTTAAAAAAGCTTCTGAAGAGGACCATACAAATGCCGCCTGCTTCGCCTGCATCCTCTTAAGCCATGGAGAAGAAAATGTAA |
| | TTTATGGGAAAGATGGTGTCACACCAATAAAGGATTTGACAGCCCACTTTAGGGGGGATAGATGCAAAACCCTTTTAGAGAAACCC |
| | AAACTCTTCTTCATTCAGGCTTGCCGAGGGACCGAGCTTGATGATGGCATCCAGGCCGACTCGGGGCCCATCAATGACACAGATGC |
| | TAATCCTCGATACAAGATCCCAGTGGAAGCTGACTTCCTCTTCGCCTATTCCACGGTTCCAGGCTATTACTCGTGGAGGAGCCCAGG |
| | AAGAGGCTCCTGGTTTGTGCAAGCCCTCTGCTCCATCCTGGAGGAGCACGGAAAAGACCTGGAAATCATGCAGATCCTCACCAGGG |
| | TGAATGACAGAGTTGCCAGGCACTTTGAGTCTCAGTCTGATGACCCACACTTCCATGAGAAGAAGCAGATCCCCTGTGTGGTCTCC |
| | ATGCTCACCAAGGAACTCTACTTCAGTCAATAG |
| FGFR2:CCDC6 | >ATGGTCAGCTGGGGTCGTTTCATCTGCCTGGTCGTGGTCACCATGGCAACCTTGTCCCTGGCCCGGCCCTCCTTCAGTTTAGTTGAG |
| (3423 base | GATACCACATTAGAGCCAGAAGAGCCACCAACCAAATACCAAATCTCTCAACCAGAAGTGTACGTGGCTGCGCCAGGGGAGTCGC |
| pairs) | TAGAGGTGCGCTGCCTGTTGAAAGATGCCGCCGTGATCAGTTGGACTAAGGATGGGGTGCACTTGGGGCCCAACAATAGGACAGTG |
| (SEQ ID NO: 63) | CTTATTGGGGAGTACTTGCAGATAAAGGGCGCCACGCCTAGAGACTCCGGCCTCTATGCTTGTACTGCCAGTAGGACTGTAGACAG |
| | TGAAACTTGGTACTTCATGGTGAATGTCACAGATGCCATCTCATCCGGAGATGATGAGGATGACACCGATGGTGCGGAAGATTTTG |
| | TCAGTGAGAACAGTAACAACAAGAGAGCACCATACTGGACCAACACAGAAAAGATGGAAAAGCGGCTCCATGCTGTGCCTGCGGC |
| | CAACACTGTCAAGTTTCGCTGCCCAGCCGGGGGGAACCCAATGCCAACCATGCGGTGGCTGAAAAACGGGAAGGAGTTTAAGCAG |
| | GAGCATCGCATTGGAGGCTACAAGGTACGAAACCAGCACTGGAGCCTCATTATGGAAAGTGTGGTCCCATCTGACAAGGGAAATT |
| | ATACCTGTGTAGTGGAGAATGAATACGGGTCCATCAATCACACGTACCACCTGGATGTTGTGGAGCGATCGCCTCACCGGCCCATC |
| | CTCCAAGCCGGACTGCCGGCAAATGCCTCCACAGTGGTCGGAGGAGACGTAGAGTTTGTCTGCAAGGTTTACAGTGATGCCCAGCC |
| | CCACATCCAGTGGATCAAGCACGTGGAAAAGAACGGCAGTAAATACGGGCCCGACGGGCTGCCCTACCTCAAGGTTCTCAAGGCC |
| | GCCGGTGTTAACACCACGGACAAAGAGATTGAGGTTCTCTATATTCGGAATGTAACTTTTGAGGACGCTGGGGAATATACGTGCTT |
| | GGCGGGTAATTCTATTGGGATATCCTTTCACTCTGCATGGTTGACAGTTCTGCCAGCGCCTGGAAGAGAAAAGGAGATTACAGCTTC |
| | CCCAGACTACCTGGAGATAGCCATTTACTGCATAGGGGTCTTCTTAATCGCCTGTATGGTGGTAACAGTCATCCTGTGCCGAATGAA |
| | GAACACGACCAAGAAGCCAGACTTCAGCAGCCAGCCGGCTGTGCACAAGCTGACCAAACGTATCCCCCTGCGGAGACAGGTAACA |
| | GTTTCGGCTGAGTCCAGCTCCTCCATGAACTCCAACACCCCGCTGGTGAGGATAACAACACGCCTCTCTTCAACGGCAGACACCCCC |
| | ATGCTGGCAGGGGTCTCCGAGTATGAACTTCCAGAGGACCCAAAATGGGAGTTTCCAAGAGATAAGCTGACACTGGGCAAGCCCCT |
| | GGGAGAAGGTTGCTTTGGGCAAGTGGTCATGGCGGAAGCAGTGGGAATTGACAAAGACAAGCCCAAGGAGGCGGTCACCGTGGCC |
| | GTGAAGATGTTGAAAGATGATGCCACAGAGAAAGACCTTTCTGATCTGGTGTCAGAGATGGAGATGATGAAGATGATTGGGAAAC |
| | ACAAGAATATCATAAATCTTCTTGGAGCCTGCACACAGGATGGGCCTCTCTATGTCATAGTTGAGTATGCCTCTAAAGGCAACCTCC |
| | GAGAATACCTCCGAGCCCGGAGGCCACCCGGGATGGAGTACTCCTATGACATTAACCGTGTTCCTGAGGAGCAGATGACCTTCAAG |
| | GACTTGGTGTCATGCACCTACCAGCTGGCCAGAGGCATGGAGTACTTGGCTTCCCAAAAATGTATTCATCGAGATTTAGCAGCCAG |
| | AAATGTTTTGGTAACAGAAAACAATGTGATGAAAATAGCAGACTTTGGACTCGCCAGAGATATCAACAATATAGACTATTACAAAA |
| | AGACCACCAATGGGCGGCTTCCAGTCAAGTGGATGGCTCCAGAAGCCCTGTTTGATAGAGTATACACTCATCAGAGTGATGTCTGG |
| | TCCTTCGGGGTGTTAATGTGGGAGATCTTCACTTTAGGGGGCTCGCCCTACCCAGGGATTCCCGTGGAGGAACTTTTTAAGCTGCTG |
| | AAGGAAGGACACAGAATGGATAAGCCAGCCAACTGCACCAACGAACTGTACATGATGATGAGGGACTGTTGGCATGCAGTGCCCT |
| | CCCAGAGACCAACGTTCAAGCAGTTGGTAGAAGACTTGGATCGAATTCTCACTCTCACAACCAATGAGCAAGCCAGGGCTGAGCAG |
| | GAAGAAGAATTCATTAGTAACACTTTATTCAAGAAAATTCAGGCTTTGCAGAAGGAGAAAGAAACCCTTGCTGTAAATTATGAGAA |
| | AGAAGAAGAATTCCTCACTAATGAGCTCTCCAGAAAATTGATGCAGTTGCAGCATGAGAAAGCCGAACTAGAACAGCATCTTGAA |
| | CAAGAGCAGGAATTTCAGGTCAACAAACTGATGAAGAAAATTAAAAAACTGGAGAATGACACCATTTCTAAGCAACTTACATTAG |
| | AACAGTTGAGACGGGAGAAGATTGACCTTGAAAATACATTGGAACAAGAACAAGAAGCACTAGTTAATCGCCTCTGGAAAAGGAT |
| | GGATAAGCTTGAAGCTGAAAAGCGAATCCTGCAGGAAAAATTAGACCAGCCCGTCTCTGCTCCACCATCGCCTAGAGATATCTCCA |
| | TGGAGATTGATTCTCCAGAAAATATGATGCGTCACATCAGGTTTTTAAAGAATGAAGTGGAACGGCTGAAGAAGCAACTGAGAGCT |
| | GCTCAGTTACAGCATTCAGAGAAAATGGCACAGTATCTGGAGGAGGAACGTCACATGAGAGAAGAGAACTTGAGGCTCCAGAGGA |
| | AGCTGCAGAGGGAGATGGAGAGAAGAGAAGCCCTCTGTCGACAGCTCTCCGAGAGTGAGTCCAGCTTAGAAATGGACGACGAAAG |
| | GTATTTTAATGAGATGTCTGCACAAGGATTAAGACCTCGCACTGTGTCCAGCCCGATCCCTTACACACCTTCTCCGAGTTCAAGCAG |
| | GCCTATATCACCTGGTCTATCATATGCAAGTCACACGGTTGGTTTCACGCCACCAACTTCACTGACTAGAGCTGGAATGTCTTATTA |
| | CAATTCCCCGGGTCTTCACGTGCAGCACATGGGAACATCCCATGGTATCACAAGGCCTTCACCACGGAGAAGCAACAGTCCTGACA |
| | AATTCAAACGGCCCACGCCGCCTCCATCTCCCAACACACAGACCCCAGTCCAGCCACCTCCGCCTCCACCTCCGCCACCCATGCAGC |
| | CCACGGTCCCCTCAGCAGCCACCTCGCAGCCTACTCCTTCGCAACATTCGGCGCACCCCTCCTCCCAGCCTTAA |
| FGFR2:OFD1 | >TGGTCAGCTGGGGTCGTTTCATCTGCCTGGTCGTGGTCACCATGGCAACCTTGTCCCTGGCCCGGCCCTCCTTCAGTTTAGTTGAG |
| (5229 base | GATACCACATTAGAGCCAGAAGAGCCACCAACCAAATACCAAATCTCTCAACCAGAAGTGTACGTGGCTGCGCCAGGGGAGTCGC |
| pairs) | TAGAGGTGCGCTGCCTGTTGAAAGATGCCGCCGTGATCAGTTGGACTAAGGATGGGGTGCACTTGGGGCCCAACAATAGGACAGTG |
| (SEQ ID NO: 64) | CTTATTGGGGAGTACTTGCAGATAAAGGGCGCCACGCCTAGAGACTCCGGCCTCTATGCTTGTACTGCCAGTAGGACTGTAGACAG |
| | TGAAACTTGGTACTTCATGGTGAATGTCACAGATGCCATCTCATCCGGAGATGATGAGGATGACACCGATGGTGCGGAAGATTTTG |
| | TCAGTGAGAACAGTAACAACAAGAGAGCACCATACTGGACCAACACAGAAAAGATGGAAAAGCGGCTCCATGCTGTGCCTGCGGC |
| | CAACACTGTCAAGTTTCGCTGCCCAGCCGGGGGGAACCCAATGCCAACCATGCGGTGGCTGAAAAACGGGAAGGAGTTTAAGCAG |
| | GAGCATCGCATTGGAGGCTACAAGGTACGAAACCAGCACTGGAGCCTCATTATGGAAAGTGTGGTCCCATCTGACAAGGGAAATT |
| | ATACCTGTGTAGTGGAGAATGAATACGGGTCCATCAATCACACGTACCACCTGGATGTTGTGGAGCGATCGCCTCACCGGCCCATC |
| | CTCCAAGCCGGACTGCCGGCAAATGCCTCCACAGTGGTCGGAGGAGACGTAGAGTTTGTCTGCAAGGTTTACAGTGATGCCCAGCC |
| | CCACATCCAGTGGATCAAGCACGTGGAAAAGAACGGCAGTAAATACGGGCCCGACGGGCTGCCCTACCTCAAGGTTCTCAAGGCC |
| | GCCGGTGTTAACACCACGGACAAAGAGATTGAGGTTCTCTATATTCGGAATGTAACTTTTGAGGACGCTGGGGAATATACGTGCTT |
| | GGCGGGTAATTCTATTGGGATATCCTTTCACTCTGCATGGTTGACAGTTCTGCCAGCGCCTGGAAGAGAAAAGGAGATTACAGCTTC |
| | CCCAGACTACCTGGAGATAGCCATTTACTGCATAGGGGTCTTCTTAATCGCCTGTATGGTGGTAACAGTCATCCTGTGCCGAATGAA |
| | GAACACGACCAAGAAGCCAGACTTCAGCAGCCAGCCGGCTGTGCACAAGCTGACCAAACGTATCCCCCTGCGGAGACAGGTAACA |
| | GTTTCGGCTGAGTCCAGCTCCTCCATGAACTCCAACACCCCGCTGGTGAGGATAACAACACGCCTCTCTTCAACGGCAGACACCCCC |
| | ATGCTGGCAGGGGTCTCCGAGTATGAACTTCCAGAGGACCCAAAATGGGAGTTTCCAAGAGATAAGCTGACACTGGGCAAGCCCCT |
| | GGGAGAAGGTTGCTTTGGGCAAGTGGTCATGGCGGAAGCAGTGGGAATTGACAAAGACAAGCCCAAGGAGGCGGTCACCGTGGCC |
| | GTGAAGATGTTGAAAGATGATGCCACAGAGAAAGACCTTTCTGATCTGGTGTCAGAGATGGAGATGATGAAGATGATTGGGAAAC |
| | ACAAGAATATCATAAATCTTCTTGGAGCCTGCACACAGGATGGGCCTCTCTATGTCATAGTTGAGTATGCCTCTAAAGGCAACCTCC |
| | GAGAATACCTCCGAGCCCGGAGGCCACCCGGGATGGAGTACTCCTATGACATTAACCGTGTTCCTGAGGAGCAGATGACCTTCAAG |
| | GACTTGGTGTCATGCACCTACCAGCTGGCCAGAGGCATGGAGTACTTGGCTTCCCAAAAATGTATTCATCGAGATTTAGCAGCCAG |
| | AAATGTTTTGGTAACAGAAAACAATGTGATGAAAATAGCAGACTTTGGACTCGCCAGAGATATCAACAATATAGACTATTACAAAA |
| | AGACCACCAATGGGCGGCTTCCAGTCAAGTGGATGGCTCCAGAAGCCCTGTTTGATAGAGTATACACTCATCAGAGTGATGTCTGG |
| | TCCTTCGGGGTGTTAATGTGGGAGATCTTCACTTTAGGGGGCTCGCCCTACCCAGGGATTCCCGTGGAGGAACTTTTTAAGCTGCTG |
| | AAGGAAGGACACAGAATGGATAAGCCAGCCAACTGCACCAACGAACTGTACATGATGATGAGGGACTGTTGGCATGCAGTGCCCT |
| | CCCAGAGACCAACGTTCAAGCAGTTGGTAGAAGACTTGGATCGAATTCTCACTCTCACAACCAATGAGACACAACTTCGAAACCAG |
| | CTAATTCATGAGTTGATGCACCCTGTATTGAGTGGAGAACTGCAGCCTCGGTCCATTTCAGTAGAAGGGAGCTCCCTCTTAATAGGC |
| | GCCTCTAACTCTTTAGTGGCAGATCACTTACAAAGATGTGGCTATGAATATTCACTTTCTGTTTTCTTTCCAGAAAGTGGTTTGGCAA |
| | AAGAAAAGGTATTTACTATGCAGGATCTATTACAACTCATTAAAATCAACCCTACTTCCAGTCTCTACAAATCACTGGTTTCAGGAT |
| | CTGATAAAGAAAATCAAAAAGGTTTTCTTATGCATTTTTTAAAAGAATTGGCAGAATATCATCAAGCTAAAGAGAGTTGTAATATG |
| | GAAACTCAGACAAGTTCGACATTTAACAGAGATTCTCTGGCTGAGAAGCTTCAGCTTATTGATGATCAGTTTGCAGATGCTTACCCT |
| | CAGCGTATCAAGTTCGAATCTTTAGAAATAAAGCTAAATGAGTATAAGAGAGAAATAGAAGAGCAACTTCGGGCAGAAATGTGTC |
| | AAAAGTTGAAGTTTTTTAAAGATACCGAGATAGCAAAAATTAAAATGGAAGCAAAAAAAAAGTATGAAAAGGAGTTAACCATGTT |
| | CCAGAATGATTTTGAAAAAGCTTGTCAAGCAAAATCTGAAGCTCTCGTTCTTCGGGAAAAGAGTACCCTTGAAAGAATTCACAAGC |
| | ACCAAGAGATTGAAACAAAAGAAATTTATGCTCAAAGGCAACTTTTACTAAAAGATATGGATTTGCTAAGAGGAAGAGAAGCAGA |
| | GCTGAAGCAAAGAGTTGAAGCTTTTGAATTGAACCAGAAGCTCCAGGAAGAAAAACATAAAAGCATAACTGAGGCACTTAGGAGA |
| | CAGGAGCAGAATATAAAGAGTTTTGAGGAGACCTATGACCGAAAGCTCAAGAATGAACTTCTAAAGTATCAACTTGAACTGAAGG |
| | ATGACTACATCATTAGAACTAATCGACTGATTGAAGATGAAAGGAAGAATAAAGAAAAAGCTGTTCATTTGCAAGAGGAGCTCAT |
| | AGCTATTAATTCAAAAAAGGAGGAACTCAATCAATCTGTAAATCGTGTGAAAGAACTTGAGCTTGAATTAGAGTCTGTCAAAGCCC |
| | AGTCTTTGGCAATAACAAAACAAAACCATATGCTGAATGAAAAGGTTAAAGAGATGAGTGATTATTCACTACTAAAAGAAGAGAA |
| | ACTGGAGCTTCTGGCACAAAATAAATTACTTAAACAACAACTGGAAGAGAGTAGAAATGAAAACCTGCGTCTCCTAAACCGCCTAG |
| | CTCAGCCGGCTCCTGAACTTGCAGTCTTTCAGAAAGAACTACGGAAAGCCGAAAAGGCTATAGTGGTTGAGCATGAGGAGTTCGAA |
| | AGCTGCAGGCAAGCTCTGCACAAACAACTGCAAGACGAAATTGAGCATTCTGCACAGCTGAAGGCCCAGATTCTAGGTTACAAAG |
| | CTTCTGTAAAGAGTTTAACTACTCAGGTTGCCGATTTAAAATTGCAACTGAAGCAAACTCAGACAGCCCTAGAGAATGAAGTGTAC |
| | TGCAATCCAAAGCAGTCTGTGATCGATCGTTCTGTCAATGGATTAATAAATGGCAATGTGGTGCCTTGCAATGGTGAGATAAGTGG |
| | GGATTTCTTGAACAATCCTTTTAAACAGGAAAACGTTCTAGCACGTATGGTTGCATCAAGGATCACAAATTATCCAACTGCATGGGT |
| | GGAGGGTAGTTCCCCTGATTCTGACCTTGAGTTTGTAGCCAATACTAAGGCAAGGGTCAAAGAGCTTCAGCAAGAGGCCGAACGCT |
| | TGGAAAAGGCTTTCAGAAGTTACCATCGGAGAGTCATTAAAAACTCTGCCAAAAGCCCACTAGCAGCAAAGAGCCCACCATCTCTG |
| | CACTTGCTGGAAGCCTTCAAAAACATTACTTCCAGTTCCCCGGAAAGACATATTTTTGGAGAGGACAGAGTTGTCTCTGAGCAGCCT |
| | CAAGTGGGCACACTTGAAGAAAGGAATGACGTCGTGGAAGCACTGACAGGCAGTGCAGCCTCGAGGCTCCGCGGGGGCACTTCCT |
| | CCAGACGCCTCTCTTCCACACCCCTTCCAAAAGCAAAAAGAAGCCTCGAAAGTGAAATGTATCTGGAAGGTCTGGGCAGATCACAC |
| | ATTGCTTCCCCCAGTCCTTGTCCTGACAGAATGCCCCTACCATCACCCACTGAGTCTAGGCACAGCCTCTCCATCCCTCCTGTCTCCA |
| | GCCCTCCGGAGCAGAAAGTGGGTCTTTATCGAAGACAAACTGAACTTCAAGACAAAAGTGAATTTTCAGATGTGGACAAGCTAGCT |
| | TTTAAGGATAATGAGGAGTTTGAATCATCTTTTGAATCTGCAGGGAACATGCCAAGGCAGTTGGAAATGGGCGGGCTTTCTCCTGCC |
| | GGGGATATGTCTCATGTGGACGCTGCTGCAGCTGCTGTGCCCCTCTCATATCAGCACCCAAGTGTAGATCAGAAACAAATTGAAGA |
| | ACAAAAGGAAGAAGAAAAAATACGGGAACAGCAAGTGAAAGAACGAAGGCAGAGAGAAGAAAGAAGGCAGAGTAACCTACAAG |
| | AAGTTTTAGAAAGGGAACGAAGAGAACTAGAAAAACTGTATCAGGAAAGGAAGATGATTGAAGAATCACTGAAGATTAAAATAA |
| | AAAAGGAATTAGAAATGGAAAATGAATTAGAAATGAGTAATCAAGAAATAAAAGACAAATCTGCTCACAGTGAAAATCCTTTAGA |
| | GAAATACATGAAAATCATCCAGCAGGAGCAAGACCAGGAGTCGGCAGATAAGAGCTCAAAAAAGATGGTCCAAGAAGGCTCCCTA |
| | GTGGACACGCTGCAATCTAGTGACAAAGTCGAAAGTTTAACAGGCTTTTCTCATGAAGAACTAGACGACTCTTGGTAA |

1. A method of identifying a bladder cancer patient that is responsive to treatment with a fibroblast growth factor receptor (FGFR) inhibitor, wherein the FGFR inhibitor comprises a compound having the structure of Formula (I), 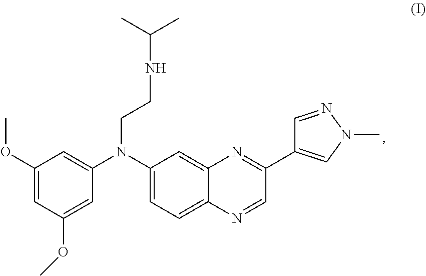 [SMILES: COc1cc(OC)cc(N(CCNC(C)C)c2ccc3ncc(-c4cnn(C)c4)nc3c2)c1]
a N-oxide thereof, a pharmaceutically acceptable salt thereof, or a solvate thereof, and wherein the method comprises:
evaluating a biological sample from the patient for a FGFR mutant from a FGFR mutant gene panel comprising FGFR3:TACC3 v1, FGFR3:TACC3 v3, FGFR3:BAIAP2L1, FGFR2:BICC1, FGFR2:AFF3, FGFR2:CASP7, FGFR3 R248C, FGFR3 S249C, FGFR3 G370C, FGFR3 Y373C, or a combination thereof, wherein said evaluating comprises:
amplifying a cDNA with a pair of primers that amplify the FGFR mutant from the FGFR mutant gene panel; and
determining whether the FGFR mutant from the FGFR mutant gene panel is present in the sample, wherein the presence of the FGFR mutant indicates that the patient is responsive to treatment with the FGFR inhibitor.

2. (canceled)

3. The method of claim 1, wherein the FGFR mutant comprises R248C, S249C, G370C, or Y373C, or any combination thereof.

4. (canceled)

5. The method of claim 1, wherein the bladder cancer is a metastatic bladder cancer.

6.-15. (canceled)

16. The method of claim 1, wherein the FGFR mutant and pair of primers are:
FGFR3:TACC3 v1 and primers having the amino acid sequences of SEQ ID NO:5 and SEQ ID NO:6;
FGFR3:TACC3 v3 and primers having the amino acid sequences of SEQ ID NO:7 and SEQ ID NO:8;
FGFR3:BAIAP2L1 and primers having the amino acid sequences of SEQ ID NO:11 and SEQ ID NO:12;
FGFR2:BICC1 and primers having the amino acid sequences of SEQ ID NO:13 and SEQ ID NO:14;
FGFR2:AFF3 and primers having the amino acid sequences of SEQ ID NO:15 and SEQ ID NO:16;
FGFR2:CASP7 and primers having the amino acid sequences of SEQ ID NO:17 and SEQ ID NO:18;
R248C and primers having the amino acid sequences of SEQ ID NO:23 and SEQ ID NO:24 or SEQ ID NO:31 and SEQ ID NO:32;
S249C and primers having the amino acid sequences of SEQ ID NO:25 and SEQ ID NO:26 or SEQ ID NO:33 and SEQ ID NO:34;
G370C and primers having the amino acid sequences of SEQ ID NO:27 and SEQ ID NO:28 or SEQ ID NO:35 and SEQ ID NO:36;
Y373C and primers having the amino acid sequences of SEQ ID NO:29 and SEQ ID NO:30 or SEQ ID NO:37 and SEQ ID NO:38;
or any combination thereof.

17. The method of claim 1, wherein the evaluating comprises: isolating an RNA from the biological sample and synthesizing a cDNA from the isolated RNA.

18. The method of claim 17, further comprising pre-amplifying the cDNA prior to the amplifying step.

19. The method of claim 1, wherein the cDNA is preamplified.

20. The method of claim 1, wherein the amplifying step comprises performing a real-time PCR.

21. The method of claim 20, wherein the real-time PCR is performed with one or more probes comprising SEQ ID NO:43, SEQ ID NO:44, SEQ ID NO:45, SEQ ID NO:47, SEQ ID NO:48, SEQ ID NO:49, SEQ ID NO:52, SEQ ID NO:53, SEQ ID NO:54, and/or SEQ ID NO:55.

22. The method of claim 21, wherein the real-time PCR is further performed with one or more 3′ blocking oligonucleotides comprising SEQ ID NO:39, SEQ ID NO:40, SEQ ID NO:41, and/or SEQ ID NO:42.

23. The method of claim 1, wherein said determining step comprises sequencing the amplified cDNA.

24.-30. (canceled)
